(19) United States
(12) Patent Application Publication
(10) Pub. No.: US 2023/0096704 A1
(43) Pub. Date: Mar. 30, 2023

(54) RNA-LOADED NANOPARTICLES AND USE THEREOF FOR THE TREATMENT OF CANCER

(71) Applicant: UNIVERSITY OF FLORIDA RESEARCH FOUNDATION, INCORPORATED, Gainesville, FL (US)

(72) Inventors: Elias Sayour, Newberry, FL (US); Hector Ruben Mendez-Gomez, Gainesville, FL (US); Sadeem Qdaisat, Gainesville, FL (US); Loic Pierre Deleyrolle, Gainesville, FL (US); Duane A. Mitchell, Gainesville, FL (US)

(21) Appl. No.: 17/797,810

(22) PCT Filed: Feb. 5, 2021
(86) PCT No.: PCT/US21/16925

§ 371 Date: Aug. 5, 2022

Related U.S. Application Data
(60) Provisional application No. 62/991,404, filed on Mar. 18, 2020.
(60) Provisional application No. 62/970,646, filed on Feb. 5, 2020.

(51) Int. Cl. A61K 39/00; A61K 9/127; C07K 14/47; A61P 35/00

(52) U.S. Cl. CPC A61K 39/001196; A61K 9/1272; C07K 14/4748; A61K 9/1278; A61P 35/00; C07K 2319/00; A61K 2039/6018; A61K 2039/5154

(57) ABSTRACT

Provided herein are compositions comprising a liposome comprising ribonucleic acid (RNA) molecules and a cationic lipid, wherein the RNA molecules bind to or encode an epitope of a nucleic acid encoding a fusion protein expressed by a tumor. The disclosure also provides a nanoparticle comprising a positively-charged surface and an interior comprising (i) a core and (ii) at least two nucleic acid layers, wherein each nucleic acid layer is positioned between a cationic lipid bilayer, and nucleic acid molecules in the nucleic acid layers comprise a sequence of a nucleic acid molecule expressed by slow-cycling cells (SCCs). Also provided herein re methods of making a nanoparticle and methods of increasing an immune response against a tumor in a subject. Methods of treating a subject with a disease are provided herein.

### CROSS-REFERENCE TO RELATED APPLICATIONS AND INCORPORATION BY REFERENCE

This application claims the benefit of priority to U.S. Provisional Patent Application No. 62/970,646, filed Feb. 5, 2020, and U.S. Provisional Patent Application No. 62/991,404, filed Mar. 18, 2020, the disclosures of which are hereby incorporated by reference in their entireties.

The following application also is incorporated by reference: International Patent Application No. PCT/US20/42606, filed Jul. 17, 2020.

### GRANT FUNDING DISCLOSURE

This invention was made with government support under grant number K08 CA199224 awarded by the National Institutes of Health, and grant number W81XWH-17-1-0510 awarded by the U.S. Army Medical Research Acquisition. The government has certain rights in the invention.

### FIELD OF THE INVENTION

The application relates to RNA-loaded nanoparticles and methods of use in, e.g., the treatment of cancer.

The Sequence Listing, which is a part of the present disclosure, is submitted concurrently with the specification as a text file. The name of the text file containing the Sequence Listing is “55331_Seqlisting.txt”, which was created on Feb. 5, 2021 and is 8,623 bytes in size. The subject matter of the Sequence Listing is incorporated herein in its entirety by reference.

### BACKGROUND

Malignant brain tumors are the most common form of cancer death in children and necessitate the development of new therapies. Immunotherapy promises to re-direct the host immune system with exquisite precision without toxicity. Immunotherapy relies on the cytotoxic potential of activated T cells, which scavenge to recognize and reject tumor associated or specific antigens (TAAs or TSAs). T cells can be ex vivo activated in co-culture with dendritic cells (DCs) presenting TAAs/TSAs or through transduction with a chimeric antigen receptor (CAR). Alternatively, T cells can be endogenously activated using cancer vaccines; but, in a randomized phase III trial for patients with primary glioblastoma (GBM), peptide vaccines targeting the tumor specific EGFRVIII surface antigen failed to mediate enhanced survival benefits over control vaccines. The EGFRVIII vaccine's failure to mediate anti-tumor efficacy highlights the challenge of therapeutic cancer vaccines.

Compared with other vaccine modalities, therapeutic cancer vaccines must induce immunologic response much more rapidly against malignancies (e.g., GBM) that are rapidly evolving. Further, many cancers, including GBMs, are highly invasive and marked by heterogeneous tumors associated with profound systemic/intratumoral suppression that stymies a nascent immunotherapeutic response and limits, if not completely blocks, vaccine effectiveness. Additionally, much of immunotherapy's promise lies in cancers with high mutational burdens. Yet, many childhood cancers like pediatric brain tumors are immunologically bland without a significant burden of mutations.

There is a need in the art for new immunotherapy options that address the aforementioned limitations.

### SUMMARY

Provided herein are data demonstrating the use of a nanoparticle (NP) (e.g., NP vaccine) that immunologically targets fusion proteins in cancers, such as pediatric brain cancers, for the treatment of such cancers. These nanoparticle vaccines in various aspects are composed of fusion protein-specific mRNA complexed into biocompatible lipid-NPs for systemic activation of dendritic cells (DCs) and induction of therapeutic T cell immunity.

The present disclosure provides a composition comprising a liposome comprising ribonucleic acid (RNA) molecules and a cationic lipid, wherein the RNA molecules bind to or encode an epitope of a nucleic acid encoding a fusion protein expressed by a tumor. In exemplary aspects, the epitope comprises a junction of the nucleic acid encoding the fusion protein. In various aspects, the epitope encodes an amino acid sequence which binds to an MHC Class II. In exemplary instances, the tumor is a solid tumor, optionally, a refractory solid tumor. In exemplary instances, the tumor is a malignant tumor. In exemplary aspects, the tumor is a brain tumor. Optionally, the tumor is a sarcoma. In exemplary aspects, the tumor is a resistant supratentorial ependymoma or a metastatic alveolar rhabdomyosarcoma. Without being bound to any particular theory, the compositions of the present disclosure are not limited to any particular fusion protein. By way of example, the fusion protein in various instances is a C11orf95-RELA fusion protein or a fusion protein described herein or in Parker and Zhang, Chin J Cancer 32(11): 594-603 (2013); Ding et al., In J Mol Sci 19(1): 177 (2018), Wener et al., Molecular Cancer 17, article number 28 (2018); Yu et al., Scientific Reports 9, article number 1074 (2019) (all of which is incorporated by reference in their entireties and particularly with respect to the disclosure relating to fusion proteins). Optionally, the composition comprises a nanoparticle comprising a positively-charged surface and an interior comprising (i) a core and (ii) at least two nucleic acid layers, wherein each nucleic acid layer is positioned between a cationic lipid bilayer. In exemplary embodiments, the nanoparticle comprises at least three nucleic acid layers, each of which is positioned between a cationic lipid bilayer. In exemplary aspects, the nanoparticle comprises at least four or five or more nucleic acid layers, each of which is positioned between a cationic lipid bilayer. In various aspects, the outermost layer of the nanoparticle comprises a cationic lipid bilayer. In various instances, the surface comprises a plurality of hydrophilic moieties of the cationic lipid bilayer. In exemplary aspects, the core comprises a cationic lipid bilayer. Optionally, the core comprises less than about 0.5 wt % nucleic acid. The diameter of the nanoparticle, in various aspects, is about 50 nm to about 250 nm in diameter, optionally, about 70 nm to about 200 nm in diameter. In exemplary instances, the nanoparticle is characterized by a zeta potential of about +40 mV to about +60 mV, optionally, about +45 mV to about +55 mV. The nanoparticle in various instances, has a zeta potential of about 50 mV. In some aspects, the nucleic acid molecules are present at a nucleic acid molecule: cationic lipid ratio of about 1 to about 5 to about 1 to about 20, optionally, about 1 to about 15, about 1 to about 10 or about 1 to about 7.5. In various aspects, the nucleic acid molecules are RNA molecules, optionally, messenger RNA (mRNA). In various aspects, the mRNA is in vitro transcribed mRNA wherein the in vitro transcription template is cDNA made from RNA extracted from a tumor cell. In various aspects, the RNA molecules are mRNA and optionally, the mRNA is in vitro transcribed mRNA wherein the in vitro transcription template is cDNA made from RNA extracted from a tumor cell. In exemplary instances, the liposomes are prepared by mixing the nucleic acid molecules and the cationic lipid at an RNA: cationic lipid ratio of about 1 to about 5 to about 1 to about 20, optionally, about 1 to about 15.

The disclosure further provides a nanoparticle comprising a positively-charged surface and an interior comprising (i) a core and (ii) at least two nucleic acid layers, wherein each nucleic acid layer is positioned between a cationic lipid bilayer, and nucleic acid molecules in the nucleic acid layers comprise a sequence of a nucleic acid molecule expressed by slow-cycling cells (SCCs), as well as compositions comprising one or more of the nanoparticles. In various aspects, the nanoparticle comprises at least three nucleic acid layers, at least four nucleic acid layers, or five or more nucleic acid layers, each of which is positioned between a cationic lipid bilayer. In various aspects, the outermost layer of the nanoparticle comprises a cationic lipid bilayer and/or the surface comprises a plurality of hydrophilic moieties of the cationic lipid of the cationic lipid bilayer and/or the core comprises a cationic lipid bilayer. Optionally, the diameter of the nanoparticle is about 50 nm to about 250 nm in diameter (e.g., about 70 nm to about 200 nm in diameter). Also optionally, the nanoparticle comprises a zeta potential of about 40 mV to about 60 mV (e.g., about 45 mV to about 55 mV, or about 50 mV). In various aspects, the nucleic acid molecules and cationic lipid are present at a ratio of about 1 to about 5 to about 1 to about 20, optionally, about 1 to about 15 or about 1 to about 7.5. Exemplary cationic lipids include DOTAP and DOTMA. In various aspects, the nucleic acid molecules are RNA molecules, such as mRNA (e.g., in vitro transcribed mRNA wherein the template is cDNA made from RNA extracted from tumor cells, such as SCCs). Optionally, the SCCs are isolated from a mixed tumor cell population obtained from a subject with a tumor (e.g., a glioblastoma). In some aspects, the nanoparticle comprises nucleic acid molecules encoded by at least one gene listed in FIG. 20 (e.g., encoded by at least or about 2, 3, 4, 5, 6, 7, 8, 9, 10, 11, 12, 13, 14, 15, 16, 17, 18, 19, or 20 genes listed in FIG. 20; encoded by more than about 50, 60, 70, 80, 90, 100 genes listed in FIG. 20; or encoded by at least or about 200, 300, 400, 500, or 600 genes listed in FIG. 20).

Also provided are methods of making a nanoparticle comprising a positively-charged surface and an interior comprising (i) a core and (ii) at least two nucleic acid layers, wherein each nucleic acid layer is positioned between a cationic lipid bilayer. In exemplary embodiments, the method comprises (A) mixing nucleic acid molecules and liposomes at a RNA: liposome ratio of about 1 to about 5 to about 1 to about 20, optionally, about 1 to about 15, to obtain a RNA-coated liposomes, wherein the liposomes are made by a process of making liposomes comprising drying a lipid mixture comprising a cationic lipid and an organic solvent by evaporating the organic solvent under a vacuum; and (B) mixing the RNA-coated liposomes with a surplus amount of liposomes, wherein the RNA bind to or encode an epitope of a nucleic acid encoding a fusion protein expressed by a tumor. In exemplary instances, the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent. Optionally, the process of making liposomes further comprises rehydrating the lipid mixture with a rehydration solution to form a rehydrated lipid mixture and then agitating, resting, and sizing the rehydrated lipid mixture. In various aspects, the method comprises sizing the rehydrated lipid mixture comprises sonicating, extruding and/or filtering the rehydrated lipid mixture.

Further provided herein are nanoparticles made by the presently disclosed method of making a nanoparticle. Additionally provided herein is a cell comprising a nanoparticle of the present disclosure. Optionally, the cell is an antigen-presenting cell (APC), e.g., a dendritic cell (DC). The present disclosure also provides a population of cells, wherein at least 50% of the population are cells according to the present disclosure.

The present disclosure provides a pharmaceutical composition comprising a plurality of nanoparticles according to the present disclosure and a pharmaceutically acceptable carrier, diluent, or excipient. In various aspects, the composition comprises about 10¹⁰ nanoparticles per mL to about 10¹⁵ nanoparticles per mL, optionally about 10¹² nanoparticles±10% per mL.

A method of increasing an immune response against a tumor in a subject is provided by the present disclosure. In exemplary embodiments, the method comprises administering to the subject the pharmaceutical composition of the present disclosure. In exemplary aspects, the nucleic acid molecules are mRNA. Optionally, the composition is systemically administered to the subject. For example, the composition is administered intravenously. In various aspects, the pharmaceutical composition is administered in an amount which is effective to activate dendritic cells (DCs) in the subject. In various instances, the immune response is a T cell-mediated immune response

Further provided are methods of treating a subject with a disease. In various aspects, the method comprises administering to the subject a pharmaceutical composition of the present disclosure in an amount effective to treat the disease in the subject. In various instances, the subject has cancer or a tumor. Optionally, the pharmaceutical composition is administered intravenously to the patient. In exemplary aspects, the patient is a pediatric patient.

### BRIEF DESCRIPTION OF THE DRAWINGS

FIG. 1A is a series of illustrations of a lipid bilayer, liposome and a general scheme leading to multilamellar (ML) RNA NPs (boxed).

FIG. 1B is a pair of CEM images of uncomplexed NPs (left) and ML RNA NPs (right).

FIG. 2A is an illustration of a general scheme leading to cationic RNA lipoplexes.

FIG. 2B is an illustration of a general scheme leading to cationic RNA lipoplexes.

FIG. 2C is a CEM image of uncomplexed NPs, FIG. 2D is a CEM image of RNA LPXs, and FIG. 2E is a CEM image of ML RNA NPs.

FIG. 2F is a graph of the % CD86+ of CD11c+MHC Class II+ splenocytes present in the spleens of mice treated with ML RNA NPs (ML RNA-NPs), RNA LPXs, anionic LPXs, or of untreated mice.

FIG. 2G is a graph of the % CD44+CD62L+ of CD8+ splenocytes present in the spleens of mice treated with ML RNA NPs (ML RNA-NPs), RNA LPXs, anionic LPXs, or of untreated mice.

FIG. 2H is a graph of the % CD44+CD62L of CD4+ splenocytes present in the spleens of mice treated with ML RNA NPs (ML RNA-NPs), RNA LPXs, anionic LPXs, or of untreated mice.

FIG. 2I is a graph of the % survival of mice treated with ML RNA NPs (ML RNA-NPs), RNA LPXs, anionic LPXs, or of untreated mice.

FIG. 2J is a graph of the amount of IFN-α produced in mice upon treatment with ML RNA NPs (ML RNA-NPs), RNA LPXs, anionic LPXs, or of untreated mice.

FIG. 3A is a pair of photographs of lungs of mice treated with ML RNA NPs or of untreated mice.

FIG. 3B is a graph of the % central memory T cells (CD62L+CD44+ of CD3+ cells) present in mice treated with ML RNA NPs loaded with tumor specific RNA or with ML RNA NPs with non-specific RNA (GFP RNA) or of untreated mice.

FIG. 3C is a graph of the % survival of mice treated with ML RNA NPs loaded with tumor specific RNA or with ML RNA NPs with non-specific RNA (GFP RNA) or of untreated mice.

FIG. 3D is a graph of the % survival of mice treated with ML RNA NPs loaded with tumor specific RNA or with ML RNA NPs with non-specific RNA (GFP RNA) or of untreated mice. This model is different from the one used to obtain the data of FIG. 3C.

FIG. 4A is a graph of the % expression of CD8 or CD44 and CD8 of CD3+ cells plotted as a function of time post administration of ML RNA NPs. FIG. 4B is a graph of the % expression of PDL1, MHC II, CD86 or CD80 of CD11c+ cells plotted as a function of time post administration of ML RNA NPs. FIG. 4C is a graph of the % expression of CD44 and CD8 of CD3+ cells plotted as a function of time post administration of ML RNA NPs. FIG. 4D is a graph of the % survival of a canine treated with ML RNA NPs compared to the median survival (dotted line).

FIG. 5 is a CEM image of ML RNA NPs and point to examples with several layers.

FIG. 6 is a cartoon delineating the generation of personalized tumor mRNA loaded NPs: From as few as 100-500 biopsied brain tumor cells, total RNA is extracted and a cDNA library is generated from which copious amounts of mRNA (representing a personalized tumor specific transcriptome) can be amplified. Negatively charged tumor mRNA is then encapsulated into positively charged lipid NPs. NPs encapsulate RNA through electrostatic interaction and are administered intravenously (iv) for uptake by dendritic cells (DCs) in reticuloendothelial organs (i.e. liver spleen and lymph nodes). The RNA is then translated and processed by a DC's intracellular machinery for presentation of peptides onto MHC Class I and II molecules, which activate CD4 and CD8+ T cells.

FIG. 7A is a timeline of the long-term survivor treatment. First and Second tumor inoculations are shown. FIG. 7B is a graph of the percent survival of animals after the second tumor inoculation for each of the three groups of mice: two groups treated before 2^{nd} tumor inoculation with ML RNA NPs comprising non-specific RNA (RNA not specific to the tumor in the subject; Green Fluorescence Protein (GFP) or pp65) and one group treated before 2^{nd} tumor inoculation with ML RNA NPs comprising tumor specific RNA or untreated animals prior to 2^{nd} tumor inoculation. Control group survival percentage is noted as “Untreated”.

FIG. 8 is a series of images depicting the localization of anionic LPX in mice upon administration.

FIG. 9A: Mouse model of glioma. PCA analysis of RNA sequencing data shows differential expression between slow vs fast-cycling cells in a mouse model of glioma. Control represents adult mouse normal astrocytes.

FIG. 9B: RNA sequencing was performed with tumor cells from nine different glioblastoma (GBM) patients and revealed differential gene-specific expression and transcript abundance between glioma slow- and fast-cycling cells. Differentially expressed genes derived from limma-voom algorithm were visualized by p-heatmap.

FIGS. 10A-10B. NP complexes were generated using RNA derived from total unselected KR158B tumor cells (TTRNA-NP), fast-cycling cells (Fast RNA-NP) and slow-cycling cells (Slow RNA-NP). Empty NP (NP alone) were used as negative control. The different NP vaccines were injected every 4-5 days for a total of three vaccines into naïve C57Bl/6 mice. T cells were then isolated from spleens and co-cultured with unselected KR158B-GFP tumor cells. FIG. 10A) After 48 h of co-culture, tumor cell death was measured by flow cytometry using GFP and propidium iodide incorporation rate. Greatest anti-tumor activity was measured with T cells derived from animals vaccinated with slow-cycling cell RNA-NP. FIG. 10B) Light microscopy images of the co-cultures reveal in the slow-cycling vaccine group a greater proliferation of T cells surrounding tumor cells represented by a white star.

FIGS. 11A-11B. Superior anti-tumor activity from RNA slow-cycling based vaccines. KR158B cells expressing luciferase were implanted intracranially. Tumor bearing animals were vaccinated with the following RNA-NP vaccines: empty NP (control), total (TT) RNA-NP (unselected), fast RNA-NP (fast) and slow RNA-NP (slow). FIG. 11A) Lower tumorigenicity in the RNA-NP slow-cycling cells group was demonstrated using Xenogen IVIS imager 7 days post implant. FIG. 11B) Tumor growth was significantly reduced in the animals treated with slow-cycling RNA-NP.

FIGS. 12-13 demonstrate slow-cycling RNA-NPs mediate antigen specific T cell activity with increased TILs. Spleens and tumors were harvested from mice vaccinated with slow fast or total RNA-NPs or NPs alone one week after 3 weekly i.v. injections into C57Bl/6 mice implanted with intracranial KR158b-luc cells. (FIG. 12) Splenocytes were restimulated ex vivo with KR158B-luc cells or left unstimulated for 48 hrs in culture; supernatants were than harvested and analyzed for IFN-gamma by ELISA. (FIG. 13) Tumors were processed for analysis of effector/memory T cells (CD44+/CD62L-).

FIG. 14A. RNA sequencing analysis performed using a mouse model of glioma (KR158) revealed significant differences in the RNA population between slow- and fast-cycling glioma cells. Interestingly pathways related to immune responses and processes were found to be differentially regulated between slow- and fast-cycling cells both in vitro and vivo.

FIG. 14B. A unique immune response signature specific to the slow-cycling glioma cells commonly identified in vitro and in vivo.

FIG. 15A. An enrichment plot and graph showing the majority of the genes composing the signature were also over-expressed by human slow-cycling glioma cells identified in 9 glioblastoma patients.

FIG. 15B. Glioblastoma patients overexpressing this gene set demonstrated shorter survival, demonstrating the clinical relevance of this signature.

FIG. 16 is a graph of the SNPs difference for SCCs and non SCCs in a mouse, Patient L0 and Patient L1.

FIG. 17A is a graph of the number of MHC I high affinity neoantigens for HMCs and non-HMCs in hGBM.

FIG. 17B is a graph of the number of MHC I high affinity neoantigens for HMCs and non-HMCs in a mouse.

FIG. 18A is a spectra showing the lipid content of control cells, FCCs and SCCs. All cells were stained with a 1/1000 dilution of LipidSpot 610.

FIG. 18B is a spectra showing the lipid content of control cells, FCCs and SCCs. All cells were stained with a 1/500 dilution of LipidSpot 610.

FIG. 19A provides Kaplan-Meier survival curves showing the % survival of animals treated with control RNA-NP (GFP), RNA NP vaccines comprising RNA from SCCs or from FCCs.

FIG. 19B is a graph of the median survival among animals treated with control RNA-NP (GFP), RNA NP vaccines comprising RNA from SCCs or from FCCs

FIG. 20 is a table listing the top 620 genes that are representative of the SCC transcriptome.

FIG. 21A is a table listing multiple epitopes predicted to be strongly immunogenic based on MHC class II binding.

FIG. 21B is an illustration of a western blot using an anti-CKB antibody. The CKB band in the control (lower band in Control lane) has a size of ˜42-45 KDa, as predicted. The CKB band in GL261 was higher in the gel than control (lower band in GL261 lane), suggesting the presence of the fusion protein. B-Tubulin antibody (upper bands in control and GL261 lines) was used as loading control.

FIG. 22A describes current fusion-based tumor diagnosis and current treatment effectiveness, e.g., Ewing's Sarcoma (Onco Targets Ther. 2019; 12: 2279-2288. doi: 10.2147/OTT.S170585.). Generated using http://www.cydas.org/Resources/index.html modified by SQ.

FIG. 22B provides examples of conventional chemotherapy regimens for Ewing sarcoma. Van Matter et al., Onco Targets Ther. 2019; 12: 2279-2288.

FIG. 23 is an illustration of limitations of current immune-based therapies.

FIG. 24 is an illustration of an exemplary method for preparing RNA-NP vaccines for cancers associated with fusion proteins. Panel (a) illustrates a method of identifying a particular gene-fusion sequence for targeting using RNA-NPs. In this example, primers are generated to flank the predicted fusion gene (here, EWSR1-FL1) to amplify the fusion protein coding sequence, confirming that the predicted fusion protein is present in the subject sample. Panel (b) is a DNA electrophoresis prepared using various cancer cell lines, EWS1, EWS2, clear cell carcinoma, and non-small cell lung carcinoma, and illustrates a means for identifying fusion proteins associated with particular cancers. Lane 1: Ladder; Lane 2: EWS1, Lane 3: EWS2, Lane 4: clear cell carcinoma, Lane 5: non-small cell lung carcinoma; Lane 6; Ladder. In panel (a), EWS1 and EW2 are associated with fusion sequences similar, but not identical in size. In a clinical setting, such a method can be used to identify fusions associated with cancers and detect differences in fusions among different subjects (when compared to a reference) that may indicate that a subject would benefit for a personalized RNA-NP treatment. Panel (c) illustrates a step of sequencing the amplified gene-fusion sequence, thereby allowing identifications of any mutations contained in the fusion from the particular subject compared to a reference or a library of fusions associated with an “off the shelf” vaccine (i.e., an RNA-NP vaccine that is not specifically prepared for a particular subject). Panel (d) illustrates the option to use the identified fusion sequence to produce mRNA-NP. Panel (e) illustrates the options of administering the subject an “off the shelf” vaccine or generation of a personalized vaccine based on the mutant fusion sequence identified from the subject's sample. Panel (f) illustrates an example of a mutant fusion sequence identified in a subject, which may prompt the generation of a personalized RNA-NP vaccine specific to the mutant fusion protein (Panel g). It will be appreciated that the steps described above are illustrative and may be modified as appropriate for a particular subject.

FIG. 25 is a listing of fusion proteins described in, e.g., Ding et al., In J Mol Sci 19(1): 177 (2018) and Wener et al., Molecular Cancer 17, article number 28 (2018). Abbreviations: LLC, Lewis lung carcinoma; CC, colon carcinoma; BC, breast carcinoma; OC, ovarian carcinoma; MM, multiple myeloma; MC, mammary carcinoma; PC, prostate carcinoma; CLL, chronic lymphocytic leukemia; MZL, marginal zone lymphoma; MCL, mantle cell lymphoma; HUVEC, human umbilical vein endothelial cell; HCC, hepatocellular carcinoma; ESFT, Ewing's sarcoma family of tumors; DSRCT, Desmoplastic small round cell tumor

FIG. 26 is a listing of fusion proteins described in, e.g., Parker and Zhang, Chin J Cancer 32(11): 594-603 (2013) and Yu et al., Scientific Reports 9, article number 1074 (2019). Abbreviations: BCR, breakpoint cluster region; ABL1, Abelson murine leukemia viral oncogene homolog 1; CML, chronic myelogenous leukemia; EWSR1, Ewing sarcoma breakpoint region 1; ETS, E-twenty six; FLI1, friend leukemia virus integration 1; IGH, immunoglobulin heavy chain; SSX, synovial sarcoma X chromosome breakpoint; PML, promyelocytic leukemia; RARA, retinoic acid receptor alpha; ATF1, activating transcription factor 1; ETV, ETS variant gene; NTRK3, neurotrophic tyrosine receptor kinase, type 3; PAX8, paired box gene 8; PPARG, peroxisome proliferator-activated receptor gamma; MECT1, mucoepidermoid carcinoma translocated 1; MAML2, mastermind-like protein 2; TMPRSS2, transmembrane protease, serine 2; ERG, ETS-related gene; EML4, echinoderm microtubule-associated protein-like 4; ALK, anaplastic lymphoma receptor tyrosine kinase; NFIB, nuclear factor 1 B-type; ESRRA, estrogen receptor related alpha; FGFR3, fibroblast growth factor receptor 3; GBM, glioblastoma multiforme; BC, bladder cancer TACC3, transforming acidic coiled-coil containing protein 3; PTPRK, protein tyrosine phosphatase receptor type K; RSPO3, R-spondin family protein 3; EIF3E3, eukaryotic translation initiation factor 3, subunit E gene; RT-PCR, reverse transcription polymerase chain reaction; FGFR3-AF4/FMR2 Family, member 3 (AFF3); FGFR2-caspase 7 (CASP7); FGFR2-coiled-coil domain containing 6 (CCDC6); FGFR1-endoplasmic reticulum lipid raft-associated 2 (ERLIN2); FGFR3-BAI1-associated protein 2-like 1 (BAIAP2L1); mucoepidermoid carcinoma translocated 1 (MECT1)—Notch coactivator mastermind-like protein 2 (MAML2).

FIG. 27 is a listing of fusion proteins described in, e.g., cancer.sanger.ac.uk/cosmic/fusion.

FIG. 28 is a listing of fusion proteins associated with various cancer types.

### DETAILED DESCRIPTION

The present disclosure relates to nanoparticles comprising a cationic lipid and nucleic acids, e.g., RNA (such as mRNA) useful in generating an immune response against cancer cells. As used herein the term “nanoparticle” refers to a particle that is less than about 1000 nm in diameter. As the nanoparticles of the present disclosure comprise cationic lipids that have been processed to induce liposome formation, the presently disclosed nanoparticles in various aspects comprise liposomes. Liposomes are artificially-prepared vesicles which, in exemplary aspects, are primarily composed of a lipid bilayer or multiple lipid bilayers. In various aspects, the liposomes of the present disclosure are of different sizes and the composition may comprise one or more of (a) a multilamellar vesicle (MLV) which may be, e.g., hundreds of nanometers in diameter and may contain a series of concentric bilayers separated by narrow aqueous compartments, (b) a small unicellular vesicle (SUV) which may be, e.g., smaller than 50 nm in diameter, and (c) a large unilamellar vesicle (LUV) which may be, e.g., between 50 and 500 nm in diameter. Liposomes in various instances are designed to comprise opsonins or ligands in order to improve the attachment of liposomes to unhealthy tissue or to activate events such as, but not limited to, endocytosis. In exemplary aspects, liposomes contain a low or a high pH in order to improve the delivery of the pharmaceutical formulations. In various instances, liposomes are formulated depending on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products. Accordingly, provided herein are nanoparticles comprising liposomes comprising cationic lipids and nucleic acid molecules (e.g., RNA molecules) which are useful in eliciting an immune response against cancer cells, nucleic acid molecules which bind to or encode an epitope of a nucleic acid encoding a fusion protein expressed by a tumor or nucleic acid molecules which comprise a sequence of a nucleic acid molecule expressed by slow-cycling cells (SCCs).

In exemplary embodiments, the nanoparticle of the present disclosure comprises a surface and an interior comprising (i) a core and (ii) a nucleic acid layer. In exemplary embodiments, the nanoparticle comprises a surface and an interior comprising (i) a core and (ii) at least two nucleic acid layers, optionally, more than two nucleic acid layers. In exemplary instances, each nucleic acid layer is positioned between a lipid layer, e.g., a cationic lipid layer. In exemplary aspects, the nanoparticles are multilamellar comprising alternating layers of nucleic acid and lipid. In exemplary embodiments, the nanoparticle comprises at least three nucleic acid layers, each of which is positioned between a cationic lipid bilayer. In exemplary aspects, the nanoparticle comprises at least four or at least five nucleic acid layers, each of which is positioned between a cationic lipid bilayer. In exemplary aspects, the nanoparticle comprises more than five (e.g., 6, 7, 8, 9, 10, 11, 12, 13, 14, 15, 16, 17, 18, 19, 20, or more) nucleic acid layers, each of which is positioned between a cationic lipid bilayer. As used herein the term “cationic lipid bilayer” is meant a lipid bilayer comprising, consisting essentially of, or consisting of a cationic lipid or a mixture thereof. Suitable cationic lipids are described herein. As used herein the term “nucleic acid layer” is meant a layer of the presently disclosed nanoparticle comprising, consisting essentially of, or consisting of a nucleic acid, e.g., RNA.

The unique structure of the nanoparticle of the present disclosure results in mechanistic differences in how the multilamellar nanoparticles (ML-NPs) exert a biological effect. Previously described RNA-based nanoparticles exert their effect, at least in part, through the toll-like receptor 7 (TLR7) pathway. Surprisingly, the multi-lamellar nanoparticles of the instant disclosure mediate efficacy independent of TLR7. While not wishing to be bound to any particular theory, intracellular pathogen recognition receptors (PRRs), such as MDA-5, appear more relevant to biological activity of the multi-lamellar nanoparticles than TLRs. This likely allows ML RNA-NPs to stimulate multiple intracellular PRRs (i.e., RIG-I, MDA-5) as opposed to singular TLRs (i.e., TLR7 in the endosome) culminating in greater release of type I interferons and induction of more potent innate immunity. This allows RNA-NPs to demonstrate superior efficacy with long-term survivor benefit.

In various aspects, the presently disclosed nanoparticle comprises a positively-charged surface. In some instances, the positively-charged surface comprises a lipid layer, e.g., a cationic lipid layer. In various aspects, the outermost layer of the nanoparticle comprises a cationic lipid bilayer. Optionally, the cationic lipid bilayer comprises, consists essentially of, or consists of DOTAP. In various instances, the surface comprises a plurality of hydrophilic moieties of the cationic lipid of the cationic lipid bilayer. In some aspects, the core comprises a cationic lipid bilayer. In various instances, the core lacks nucleic acids, optionally, the core comprises less than about 0.5 wt % nucleic acid.

In exemplary aspects, the nanoparticle has a diameter within the nanometer range and accordingly in certain instances are referred to herein as “nanoliposomes” or “liposomes”. In exemplary aspects, the nanoparticle has a diameter between about 50 nm to about 500 nm, e.g., about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 500 nm, about 150 nm to about 500 nm, about 200 nm to about 500 nm, about 250 nm to about 500 nm, about 300 nm to about 500 nm, about 350 nm to about 500 nm, about 400 nm to about 500 nm. In exemplary aspects, the nanoparticle has a diameter between about 50 nm to about 300 nm, e.g., about 100 nm to about 250 nm, about 110 nm±5 nm, about 115 nm±5 nm, about 120 nm±5 nm, about 125 nm±5 nm, about 130 nm±5 nm, about 135 nm±5 nm, about 140 nm±5 nm, about 145 nm±5 nm, about 150 nm±5 nm, about 155 nm±5 nm, about 160 nm±5 nm, about 165 nm±5 nm, about 170 nm±5 nm, about 175 nm±5 nm, about 180 nm±5 nm, about 190 nm±5 nm, about 200 nm±5 nm, about 210 nm±5 nm, about 220 nm±5 nm, about 230 nm±5 nm, about 240 nm±5 nm, about 250 nm±5 nm, about 260 nm±5 nm, about 270 nm±5 nm, about 280 nm±5 nm, about 290 nm±5 nm, about 300 nm±5 nm. In exemplary aspects, the nanoparticle is about 50 nm to about 250 nm in diameter. In some aspects, the nanoparticle is about 70 nm to about 200 nm in diameter.

In exemplary aspects, the nanoparticle is present in a pharmaceutical composition comprising a heterogeneous mixture of nanoparticles ranging in diameter, e.g., about 50 nm to about 500 nm or about 50 nm to about 250 nm in diameter. Optionally, the pharmaceutical composition comprises a heterogeneous mixture of nanoparticles ranging from about 70 nm to about 200 nm in diameter.

In exemplary instances, the nanoparticle is characterized by a zeta potential of about +40 mV to about +60 mV, e.g., about +40 mV to about +55 mV, about +40 mV to about +50 mV, about +40 mV to about +50 mV, about +40 mV to about +45 mV, about +45 mV to about +60 mV, about +50 mV to about +60 mV, about +55 mV to about +60 mV. In exemplary aspects, the nanoparticle has a zeta potential of about +45 mV to about +55 mV. The nanoparticle in various instances, has a zeta potential of about +50 mV. In various aspects, the zeta potential is greater than +30 mV or +35 mV. The zeta potential is one parameter which distinguishes the nanoparticles of the present disclosure and those described in Sayour et al., Oncoimmunology 6(1): e1256527 (2016).

In exemplary embodiments, the nanoparticles comprise a cationic lipid. In some embodiments, the cationic lipid is a low molecular weight cationic lipid such as those described in U.S. Patent Application No. 20130090372, the contents of which are herein incorporated by reference in their entirety. The cationic lipid in exemplary instances is a cationic fatty acid, a cationic glycerolipid, a cationic glycerophospholipid, a cationic sphingolipid, a cationic sterol lipid, a cationic prenol lipid, a cationic saccharolipid, or a cationic polyketide. In exemplary aspects, the cationic lipid comprises two fatty acyl chains, each chain of which is independently saturated or unsaturated. In some instances, the cationic lipid is a diglyceride. For example, in some instances, the cationic lipid may be a cationic lipid of Formula I or Formula II:

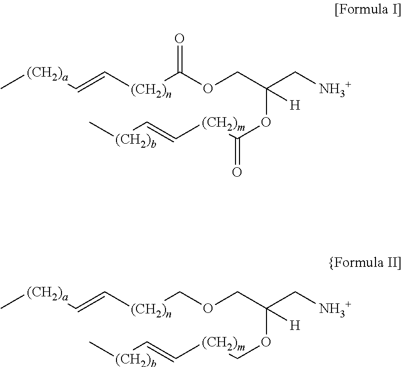

wherein each of a, b, n, and m is independently an integer between 2 and 12 (e.g., between 3 and 10). In some aspects, the cationic lipid is a cationic lipid of Formula I wherein each of a, b, n, and m is independently an integer selected from 3, 4, 5, 6, 7, 8, 9, and 10. In exemplary instances, the cationic lipid is DOTAP (1,2-dioleoyl-3-trimethylammonium-propane), or a derivative thereof. In exemplary instances, the cationic lipid is DOTMA (1,2-di-O-octadecenyl-3-trimethylammonium propane), or a derivative thereof.

In some embodiments, the nanoparticles comprise liposomes formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)[1,3]-dioxolane (DLin-KC2-DMA), and MC3 (US20100324120; herein incorporated by reference in its entirety). In some embodiments, the nanoparticles comprise liposomes formed from the synthesis of stabilized plasmid-lipid particles (SPLP) or stabilized nucleic acid lipid particle (SNALP) that have been previously described and shown to be suitable for oligonucleotide delivery in vitro and in vivo. The nanoparticles in some aspects are composed of 3 to 4 lipid components in addition to the nucleic acid molecules. In exemplary aspects, the liposome comprises 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al., Pharm Res. 2005; 22(3):362-72. In exemplary instances, the liposome comprises 48% cholesterol, 20% DSPC, 2% PEG-c-DMA, and 30% cationic lipid, where the cationic lipid can be 1,2-distearloxy-N, N-dimethylaminopropane (DSDMA), DODMA, DLin-DMA, or 1,2-dilinolenyloxy-3-dimethylaminopropane (DLenDMA), as described by Heyes et al., J. Control Release 2005; 107(2): 276-87.

In some embodiments, the liposomes comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some embodiments, the liposomes may comprise a percentage of cholesterol selected from the group consisting of 28.5%, 31.5%, 33.5%, 36.5%, 37.0%, 38.5%, 39.0% and 43.5%. In some embodiments, the liposomes may comprise from about 5.0% to about 10.0% DSPC and/or from about 7.0% to about 15.0% DSPC.

In some embodiments, the liposomes are DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In various instances, the cationic lipid comprises 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

The liposome in various aspects comprises DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some aspects, the liposome comprises a cationic lipid such as, but not limited to, DLin-DMA, DLin-D-DMA, DLin-MC3-DMA, DLin-KC2-DMA, DODMA and amino alcohol lipids. The amino alcohol cationic lipid comprises in some aspects lipids described in and/or made by the methods described in U.S. Patent Publication No. US20130150625, herein incorporated by reference in its entirety. As a non-limiting example, the cationic lipid in certain aspects is 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

In various embodiments, the liposome comprises (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid: 5-25% neutral lipid: 25-55% sterol; 0.5-15% PEG-lipid.

In some embodiments, the liposome comprises from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 94(4-(dimethylamino)butanoyl)oxy)heptadecanedioate (L319), e.g., from about 35 to about 65%, from about 45 to about 65%, about 60%, about 57.5%, about 50% or about 40% on a molar basis.

In some embodiments, the liposome comprises from about 0.5% to about 15% on a molar basis of a neutral lipid, e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In various aspects, the nanoparticle does not comprise a neutral lipid. In some embodiments, the formulation includes from about 5% to about 50% on a molar basis of the sterol (e.g., about 15 to about 45%, about 20 to about 40%, about 40%, about 38.5%, about 35%, or about 31% on a molar basis). An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis). In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005), the contents of which are herein incorporated by reference in their entirety)

In exemplary aspects, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N,N-dimethylpentacosa-16, 19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N, N-dimethylnonacos-17-en-10-amine, (24Z)—N, N-dimethyltritriacont-24-en-10-amine, (20Z)—N, N-dimethylnonacos-20-en-10-amine, (22Z)—N, N-dimethylhentriacont-22-en-10-amine, (16Z)—N, N-dimethylpentacos-16-en-8-amine, (12Z,15Z)—N, N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N, N-dimethyl-1-[(1S,2R)-2-octylcyclopropyl]hexadecan amine, N,N-dimethyl-[(1R,2S)-2-undecylcyclopropyl]tetradecan-5-amine, N,N-dimethyl-3-{7-[(1S,2R) octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R) octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxyl-3-[(9Z,12Z)-octadeca-9,12-dien-1-yloxy]propan-2-amine, (2R)-1-[(3,7-dimethyloctyl)oxy]-N, N-dimethyl-3-[(9Z,12Z)-octadeca-9,12-dien-1-yloxy]propan-2-amine, N, N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N, N-dimethylnonacosa-11,20,2-trien-10-amine or a pharmaceutically acceptable salt or stereoisomer thereof.

In some embodiments, the nanoparticle comprises a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the composition may comprise a lipid-polycation complex, which may further include a non-cationic lipid such as, but not limited to, cholesterol or dioleoyl phosphatidylethanolamine (DOPE).

In various aspects, the cationic liposomes optionally do not comprise a non-cationic lipid. Neutral molecules, in some aspects, may interfere with coiling/condensation of multi-lamellar nanoparticles resulting in RNA loaded liposomes greater than 200 nm in size. Cationic liposomes generated without helper molecules can comprise a size of about 70-200 nm (or less). These constructs consist essentially of a cationic lipid with negatively charged nucleic acid, and may be formulated in a sealed rotary vacuum evaporator which prevents oxidation of the particles (when exposed to the ambient environment). In this embodiment, the absence of a helper lipid optimizes mRNA coiling into tightly packaged multilamellar NPs where each NP contains a greater amount of nucleic acid per particle. Due to increased nucleic acid payload per particle, these multi-lamellar RNA nanoparticles drive significantly greater innate immune responses, which are a significant predictor of efficacy for modulating the immune system.

In some aspects, the nucleic acid molecules are present at a nucleic acid molecule: cationic lipid ratio of about 1 to about 5 to about 1 to about 25. In some aspects, the nucleic acid molecules are present at a nucleic acid molecule: cationic lipid ratio of about 1 to about 5 to about 1 to about 20, optionally, about 1 to about 15, about 1 to about 10, or about 1 to about 7.5. As used herein, the term “nucleic acid molecule: cationic lipid ratio” is meant a mass ratio, where the mass of the nucleic acid molecule is relative to the mass of the cationic lipid. Also, in exemplary aspects, the term “nucleic acid molecule: cationic lipid ratio” is meant the ratio of the mass of the nucleic acid molecule, e.g., RNA, added to the liposomes comprising cationic lipids during the process of manufacturing the ML RNA NPs of the present disclosure. In exemplary aspects, the nanoparticle comprises less than or about 10 μg RNA molecules per 150 μg lipid mixture. In exemplary aspects, the nanoparticle is made by incubating about 10 μg RNA with about 150 μg liposomes. In alternative aspects, the nanoparticle comprises more RNA molecules per mass of lipid mixture. For example, the nanoparticle may comprise more than 10 μg RNA molecules per 150 μg liposomes. The nanoparticle in some instances comprises more than 15 μg RNA molecules per 150 μg liposomes or lipid mixture.

In various aspects, the nucleic acid molecules are RNA molecules, e.g., transfer RNA (tRNA), ribosomal RNA (rRNA), messenger RNA (mRNA). In various aspects, the RNA molecules comprise tRNA, rRNA, mRNA, or a combination thereof. In various aspects, the RNA is total RNA isolated from a cell. In exemplary aspects, the RNA is total RNA isolated from a diseased cell, such as, for example, a tumor cell or a cancer cell, such as a slow cycling cells. Methods of obtaining total tumor RNA are known in the art and described herein at Example 1.

In exemplary instances, the RNA molecules are mRNA. In various aspects, mRNA is in vitro transcribed mRNA. In various instances, the mRNA molecules are produced by in vitro transcription (IVT). Suitable techniques of carrying out IVT are known in the art. In exemplary aspects, an IVT kit is employed. In exemplary aspects, the kit comprises one or more IVT reaction reagents. As used herein, the term “in vitro transcription (IVT) reaction reagent” refers to any molecule, compound, factor, or salt, which functions in an IVT reaction. For example, the kit may comprise prokaryotic phage RNA polymerase and promoter (T7, T3, or SP6) with eukaryotic or prokaryotic extracts to synthesize proteins from exogenous DNA templates. In exemplary aspects, the RNA is in vitro transcribed mRNA, wherein the in vitro transcription template is cDNA made from RNA extracted from a tumor cell. Optionally, the nanoparticle comprises a mixture of RNA which is RNA isolated from a tumor of a human, optionally, a malignant brain tumor, optionally, a glioblastoma, medulloblastoma, diffuse intrinsic pontine glioma, or a peripheral tumor with metastatic infiltration into the central nervous system. In various aspects, the RNA comprises a sequence encoding a poly(A) tail so that the in vitro transcribed RNA molecule comprises a poly(A) tail at the 3′ end. In various aspects, the method of making a nanoparticle comprises additional processing steps, such as, for example, capping the in vitro transcribed RNA molecules.

The mRNAs in exemplary aspects encode or bind to a protein. In exemplary instances, the mRNAs bind to or encode an epitope of a nucleic acid encoding a fusion protein expressed by a tumor. As used herein, the term “fusion protein” refers to a protein comprising at least a portion of an amino acid sequence of a first protein and at least a portion of an amino acid sequence of a second protein. In various aspects, a fusion protein is encoded by a single nucleic acid comprising a nucleotide sequence encoding at least a portion of an amino acid sequence of a first protein and a nucleotide sequence encoding at least a portion of an amino acid sequence of a second protein. In various instances, the nucleotide sequence encoding at least a portion of the amino acid sequence of the first protein is immediately adjacent to the nucleotide sequence encoding at least a portion of the amino acid sequence of the second protein. In various instances, the nucleotide sequence encoding at least a portion of the amino acid sequence of the first protein is upstream of or 5′ to the nucleotide sequence encoding at least a portion of the amino acid sequence of the second protein. In alternative aspects, the nucleotide sequence encoding at least a portion of the amino acid sequence of the first protein is downstream of or 3′ to the nucleotide sequence encoding at least a portion of the amino acid sequence of the second protein. In various aspects, the point at which the nucleotide sequence encoding at least a portion of the amino acid sequence of the first protein is connected to the nucleotide sequence encoding at least a portion of the amino acid sequence of the second protein is referred to as a “junction”. In various instances, the term “breakpoint” in the context of a fusion protein or fusion is used to mean “junction”.

Without being bound to any particular theory, the compositions of the present disclosure are not limited to any particular fusion protein. The fusion protein may comprise at least a portion of at least two different proteins. In exemplary aspects, the fusion protein comprises at least a portion of a tumor antigen, a co-stimulatory molecule, a cytokine, a growth factor, and/or a lymphokine. Examples of cytokines and growth factors are those that are effective in inhibiting tumor metastasis and those that have been shown to have an antiproliferative effect on at least one cell population. Such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neurotrophic factor, ciliary neurotrophic factor receptor α, cytokine-induced neutrophil chemotactic factor 1, cytokine-induced neutrophil, chemotactic factor 2 α, cytokine-induced neutrophil chemotactic factor 2β, β endothelial cell growth factor, endothelin 1, epithelial-derived neutrophil attractant, glial cell line-derived neurotrophic factor receptor α 1, glial cell line-derived neurotrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor a, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof. In exemplary aspects, the tumor antigen is an antigen derived from a viral protein, an antigen derived from point mutations, or an antigen encoded by a cancer-germline gene. In exemplary aspects, the tumor antigen is pp65, p53, KRAS, NRAS, MAGEA, MAGEB, MAGEC, BAGE, GAGE, LAGE/NY-ES01, SSX, tyrosinase, gp100/pmell7, Melan-A/MART-1, gp75/TRP1, TRP2, CEA, RAGE-1, HER2/NEU, WT1. In exemplary aspects, the co-stimulatory molecule is selected from the group consisting of CD80 and CD86.

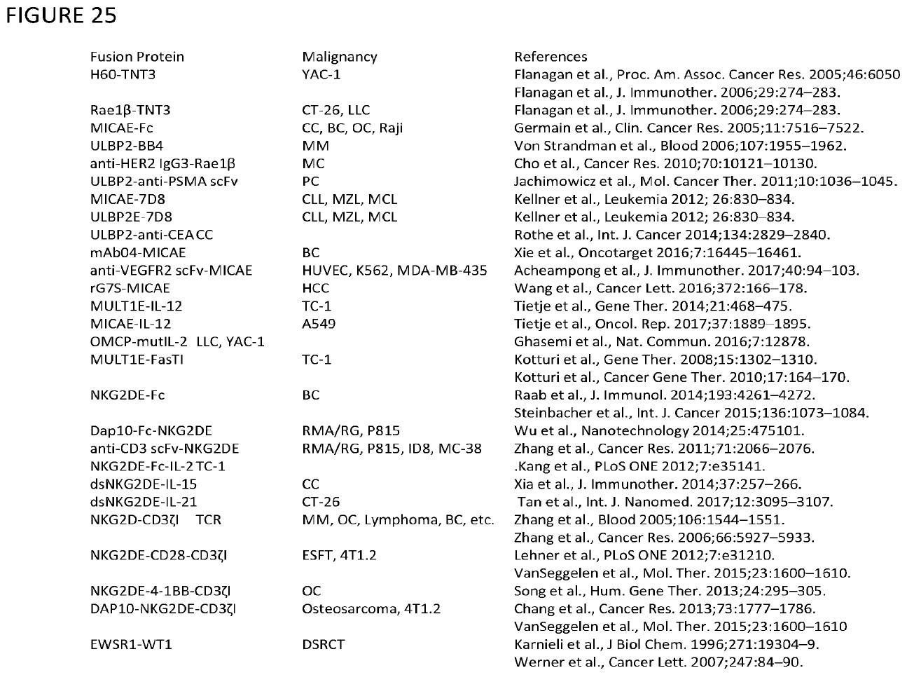

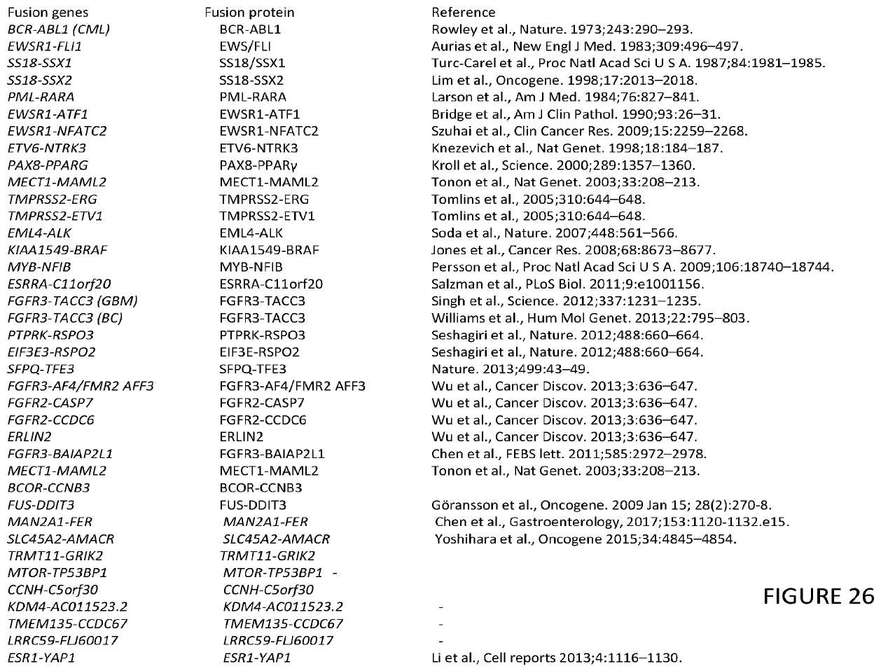

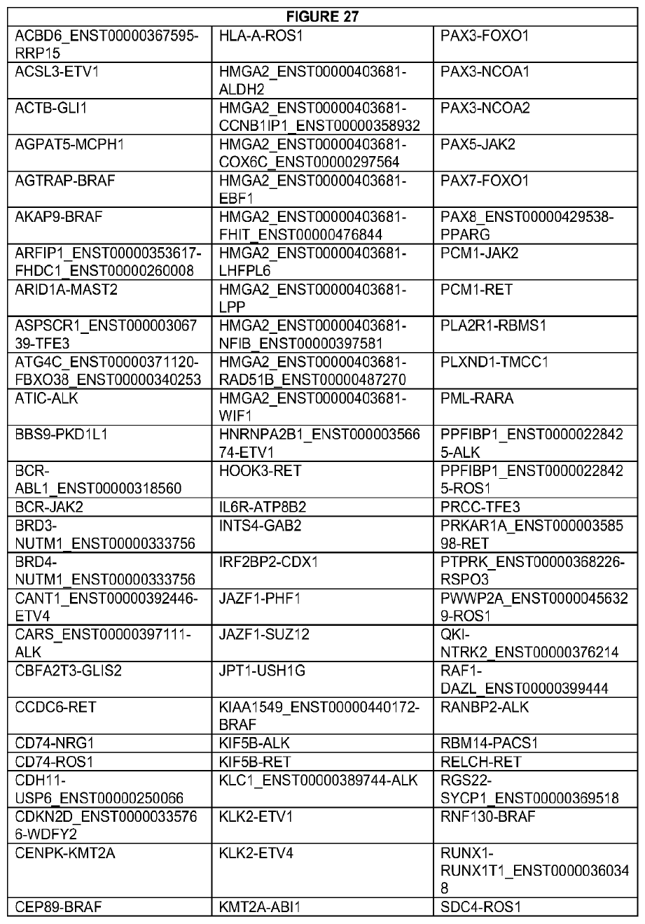

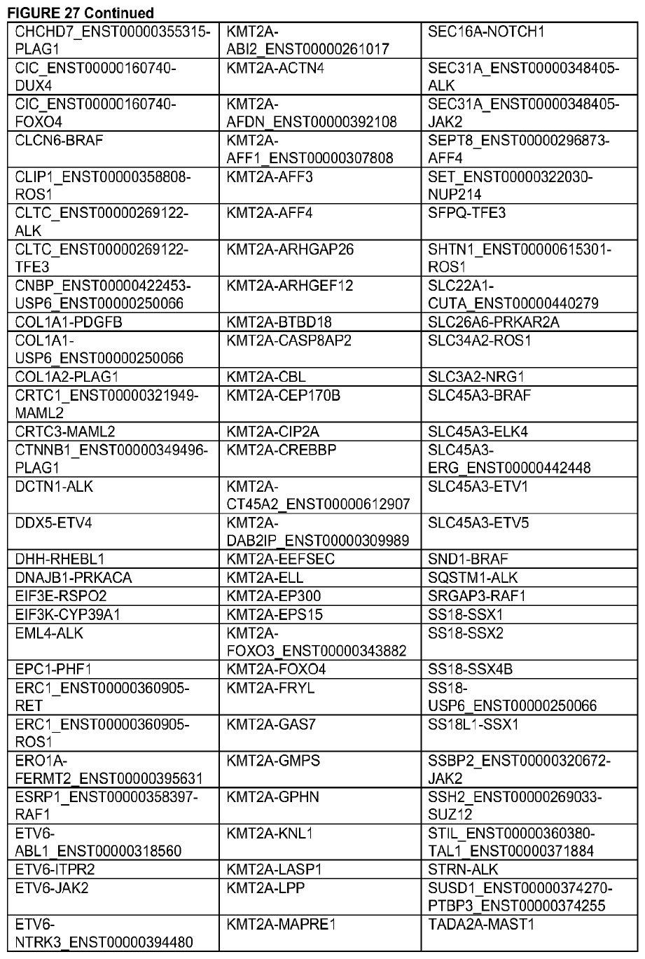

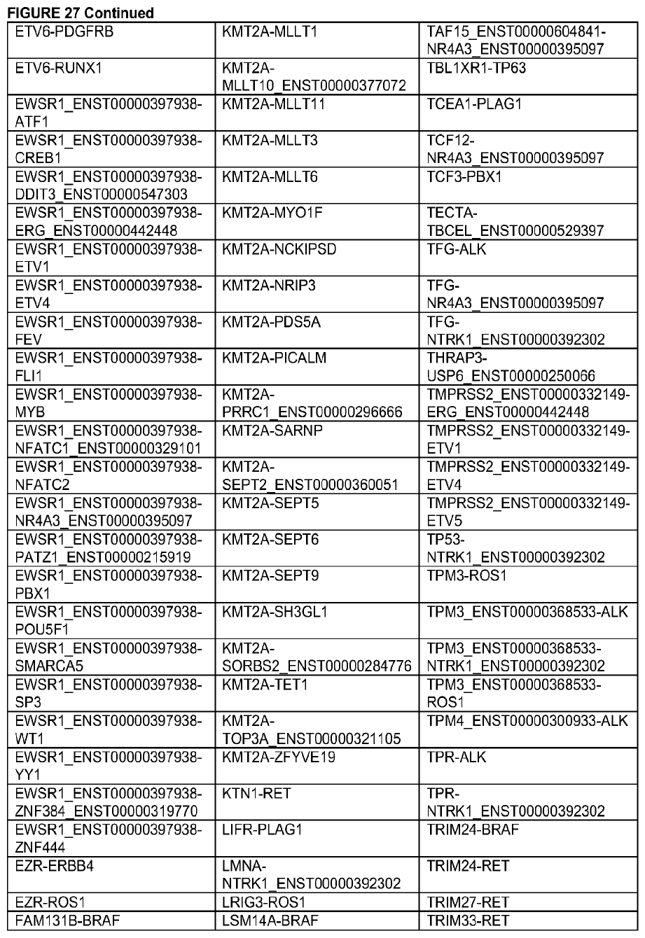

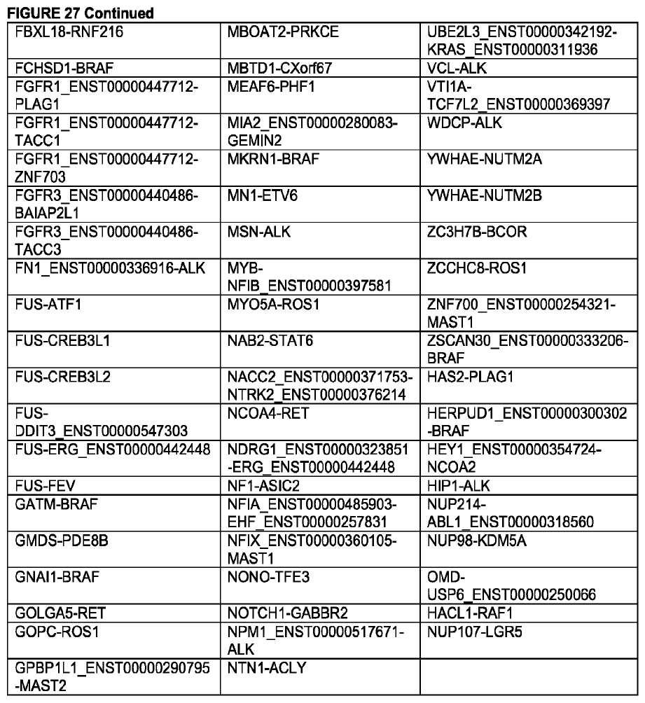

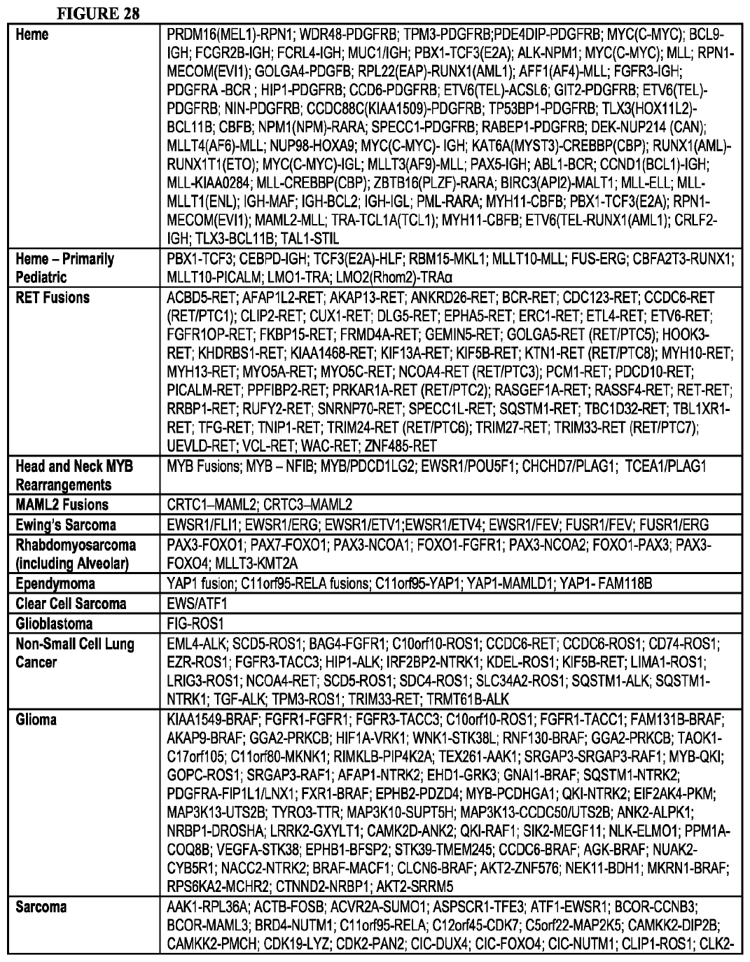

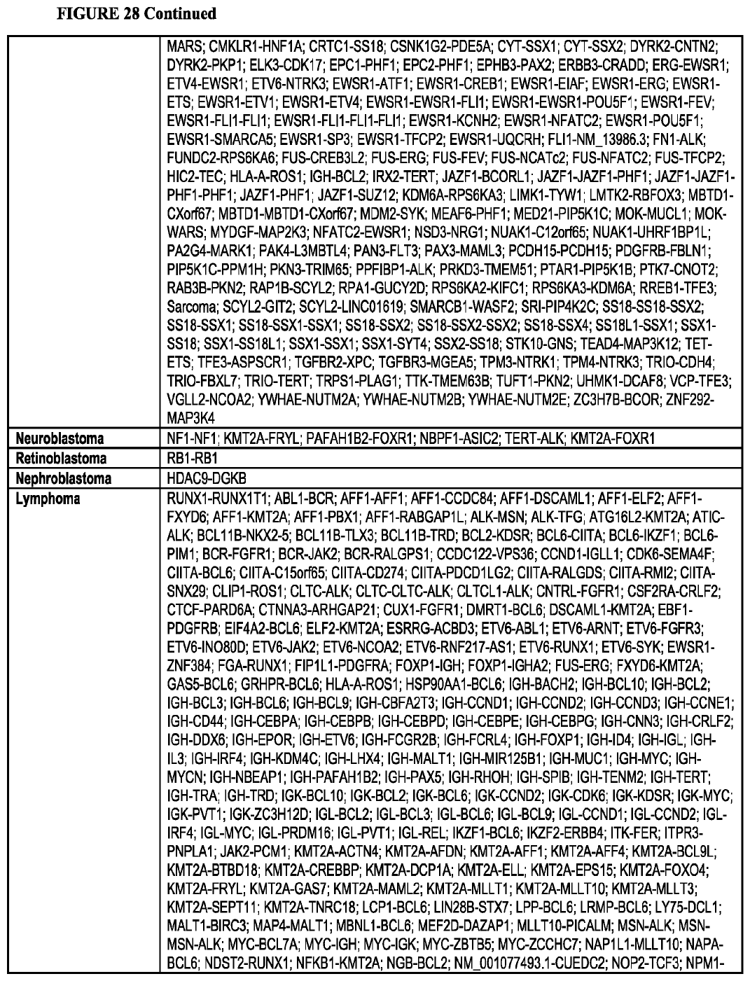

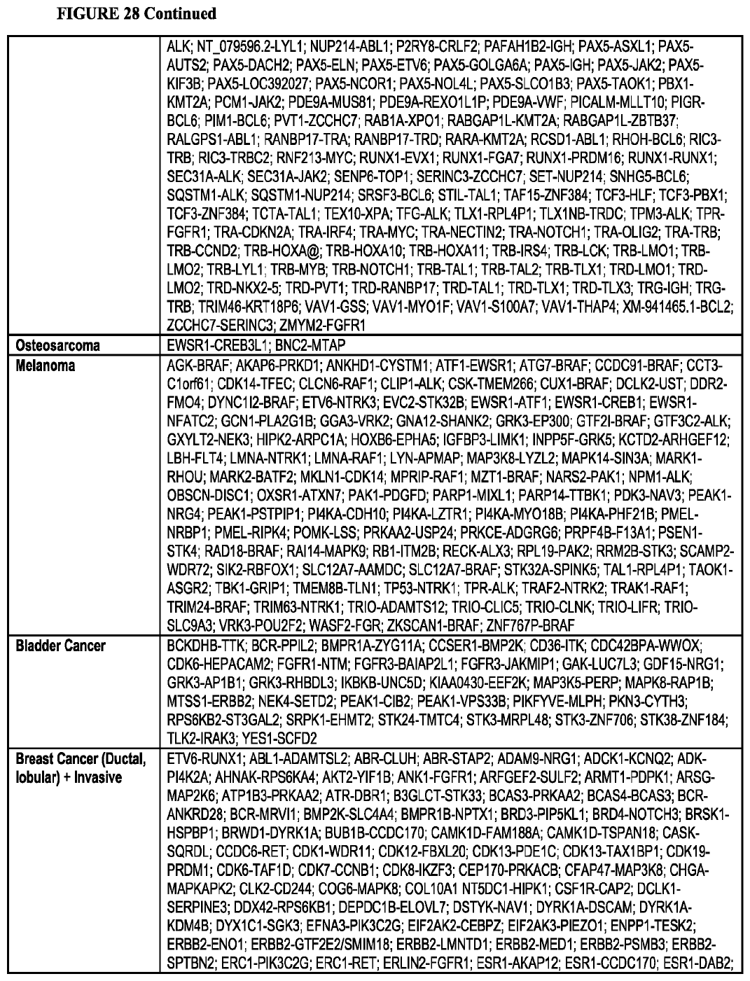

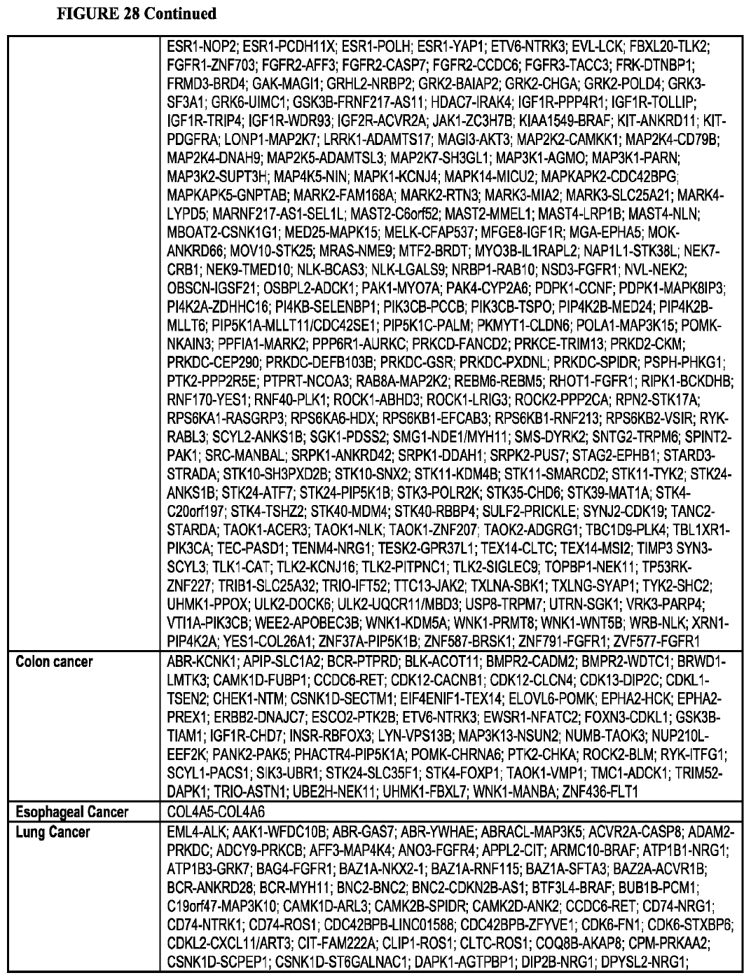

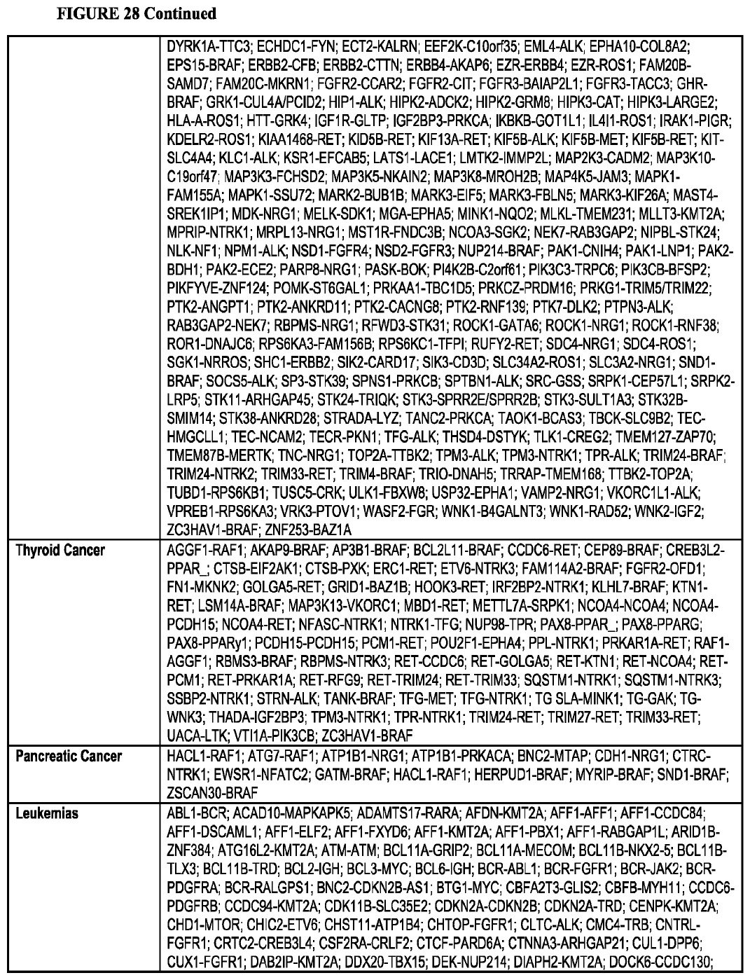

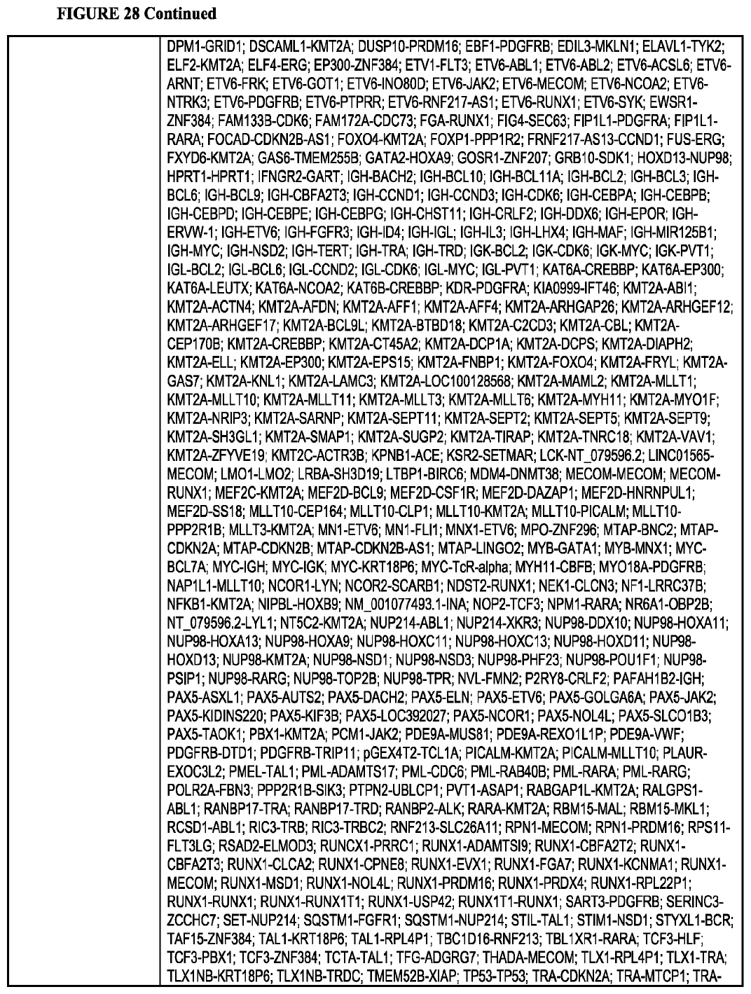

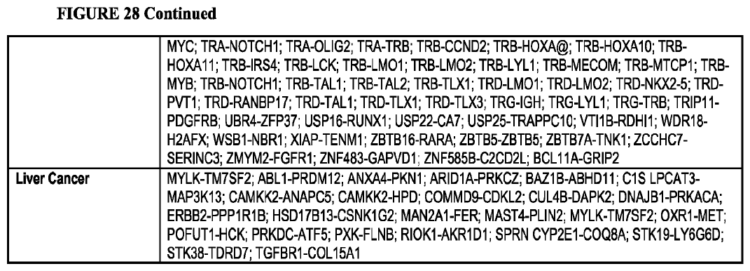

By way of example, the fusion protein in various instances is a C11orf95-RELA fusion protein or a fusion protein described herein or in Parker and Zhang, Chin J Cancer 32(11): 594-603 (2013); Ding et al., In J Mol Sci 19(1): 177 (2018); Wener et al., Molecular Cancer 17, article number 28 (2018); or Yu et al., Scientific Reports 9, article number 1074 (2019). See FIGS. 25-28. In exemplary aspects, the fusion protein is a fusion protein that comprises at least a portion of two of Erdr1, Mid1, Ppp1r13b, or CKB. In exemplary aspects, the fusion protein is a fusion protein comprising at least a portion of Erdr1 and at least a portion of Mid1 (e.g., Erdr1/Mid1 or Mid1/Erdr1) or at least a portion of Ppp1r13b and at least a portion of CKB (e.g., Ppp1r13b/CKB or CKB/Ppp1r13b). In various aspects, the fusion protein is expressed by a murine model of a brain tumor. In exemplary aspects, the fusion protein is a fusion protein comprising at least a portion of two of EWSR1, FUSR1, FOXO1, SS18, FLI1, ERG, ETV1, ETV4, FEV, SSX1. In exemplary aspects, the fusion protein comprises at least a portion of EWSR1, FUSR1, FOXO1, or SS18 and at least a portion of FLI1, ERG, ETV1, ETV4, FEV, or SSX1 (e.g., EWSR1/FLI1, FLI1/EWSR1, EWSR1/ERG, ERG/EWSR1, EWSR1/ETV1 or ETV1/EWSR1, EWSR1/ETV4, ETV4/EWSR1, EWSR1/FEV, FEV/EWSR1, FUSR1/FEV, FEV/FUSR1, FUSR1/ERG, ERG/FUSR1, FOXO1/PAX3, PAX3/FOXO1, FOXO1/PAX7, PAX7/FOXO1, SS18/SSX1, or SSX1/SS18). In exemplary aspects, the fusion protein is expressed by a sarcoma tumor. In various aspects, the fusion protein comprises at least a portion of two of YAP1, FAM118B, MAMLD1, C11or95, RELA, EPN, MTOR, CASZ1, TP53, DEK, FXR2, BRAF, KIAA1549, or EML4. In exemplary aspects, the fusion protein comprises at least a portion of YAP1, C11or95, MTOR, TP53 or BRAF and at least a portion of FAM118B, MAMLD1, RELA, C11orf95, EPN, CASZ1, DEK, FXR2, KIAA1549, or EML4 (e.g., YAP1/FAM118B, FAM118B/YAP1, YAP1/MAMLD1, MAMLD1/YAP1, YAP1/C11orf95, c11orf95/YAP1, C11orf95-RELA, c11orf95/RELA, EPN-YAP1, EPN-YAP1, MTOR/MTOR, MTOR/CASZ1, CASZ1/MTOR, TP53/TP53, TP53/DEK, DEK/TP53, TP53/FXR2, FXR2/TP53, BRAF/KIAA1549, KIAA1549/BRAF, BRAF/EML4, or EML4/BRAF). In exemplary aspects, the fusion protein is expressed by a neuro-tumor. The fusion protein may be any one of those described at the website for the Catalog of Somatic Mutations in Cancer (COSMIC) at cancer.sanger.ac.uk/cosmic/fusion or at the website for the Atlas of Genetics and Cytogenetics in Oncology and Haematology at atlasgeneticsoncology.org/Deep/Cancer_CytogenomicsID20145.html.

In exemplary aspects of the disclosure, the mRNAs within the nanoparticle encode a protein that is not a fusion protein. In these aspects, the mRNA(s) may encode any of the proteins described above. Put another way, the proteins described above are, in some embodiments, part of a fusion protein, but this is not required by all embodiments of the disclosure. The mRNA(s) may encode all or part of a tumor antigen, a co-stimulatory molecule, a cytokine, a growth factor, or a lymphokine, such as those described above. In some aspects, the protein is not expressed by a tumor cell or by a human. In exemplary instances, the protein is not related to a tumor antigen or cancer antigen. In some aspects, the protein is non-specific relative to a tumor or cancer. For example, the non-specific protein may be green fluorescence protein (GFP) or ovalbumin (OVA).

In various instances, the RNA molecules are antisense molecules, optionally siRNA, shRNA, miRNA, or any combination thereof. The antisense molecule can be one which mediates RNA interference (RNAi). RNAi is a ubiquitous mechanism of gene regulation in plants and animals in which target mRNAs are degraded in a sequence-specific manner (Sharp, Genes Dev., 15, 485-490 (2001); Hutvagner et al., Curr. Opin. Genet. Dev., 12, 225-232 (2002); Fire et al., Nature, 391, 806-811 (1998); Zamore et al., Cell, 101, 25-33 (2000)). The natural RNA degradation process is initiated by the dsRNA-specific endonuclease Dicer, which promotes cleavage of long dsRNA precursors into double-stranded fragments between 21 and 25 nucleotides long, termed small interfering RNA (siRNA; also known as short interfering RNA) (Zamore, et al., Cell. 101, 25-33 (2000); Elbashir et al., Genes Dev., 15, 188-200 (2001); Hammond et al., Nature, 404, 293-296 (2000); Bernstein et al., Nature, 409, 363-366 (2001)). siRNAs are incorporated into a large protein complex that recognizes and cleaves target mRNAs (Nykanen et al., Cell, 107, 309-321 (2001). It has been reported that introduction of dsRNA into mammalian cells does not result in efficient Dicer-mediated generation of siRNA and therefore does not induce RNAi (Caplen et al., Gene 252, 95-105 (2000); Ui-Tei et al., FEBS Lett, 479, 79-82 (2000)). The requirement for Dicer in maturation of siRNAs in cells can be bypassed by introducing synthetic 21-nucleotide siRNA duplexes, which inhibit expression of transfected and endogenous genes in a variety of mammalian cells (Elbashir et al., Nature, 411: 494-498 (2001)).

In this regard, the RNA molecule in some aspects mediates RNAi and in some aspects is a siRNA molecule specific for inhibiting the expression of a protein. The term “siRNA” as used herein refers to an RNA (or RNA analog) comprising from about 10 to about 50 nucleotides (or nucleotide analogs) which is capable of directing or mediating RNAi. In exemplary embodiments, an siRNA molecule comprises about 15 to about 30 nucleotides (or nucleotide analogs) or about 20 to about 25 nucleotides (or nucleotide analogs), e.g., 21-23 nucleotides (or nucleotide analogs). The siRNA can be double or single stranded, preferably double-stranded.

In alternative aspects, the RNA molecule is a short hairpin RNA (shRNA) molecule specific for inhibiting the expression of a protein. The term “shRNA” as used herein refers to a molecule of about 20 or more base pairs in which a single-stranded RNA partially contains a palindromic base sequence and forms a double-strand structure therein (i.e., a hairpin structure). An shRNA can be an siRNA (or siRNA analog) which is folded into a hairpin structure. shRNAs typically comprise about 45 to about 60 nucleotides, including the approximately 21 nucleotide antisense and sense portions of the hairpin, optional overhangs on the non-loop side of about 2 to about 6 nucleotides long, and the loop portion that can be, e.g., about 3 to 10 nucleotides long. The shRNA can be chemically synthesized. Alternatively, the shRNA can be produced by linking sense and antisense strands of a DNA sequence in reverse directions and synthesizing RNA in vitro with T7 RNA polymerase using the DNA as a template.

Though not wishing to be bound by any theory or mechanism it is believed that after shRNA is introduced into a cell, the shRNA is degraded into a length of about 20 bases or more (e.g., representatively 21, 22, 23 bases), and causes RNAi, leading to an inhibitory effect. Thus, shRNA elicits RNAi and therefore can be used as an effective component of the disclosure. shRNA may preferably have a 3′-protruding end. The length of the double-stranded portion is not particularly limited, but is preferably about 10 or more nucleotides, and more preferably about 20 or more nucleotides. Here, the 3′-protruding end may be preferably DNA, more preferably DNA of at least 2 nucleotides in length, and even more preferably DNA of 2-4 nucleotides in length.

In exemplary aspects, the antisense molecule is a microRNA (miRNA). The term “microRNA” refers to a small (e.g., 15-22 nucleotides), non-coding RNA molecule which base pairs with mRNA molecules to silence gene expression via translational repression or target degradation. microRNA and the therapeutic potential thereof further described in, e.g., Mulligan, MicroRNA: Expression, Detection, and Therapeutic Strategies, Nova Science Publishers, Inc., Hauppauge, N.Y., 2011; Bader and Lammers, “The Therapeutic Potential of microRNAs” Innovations in Pharmaceutical Technology, pages 52-55 (March 2011).

In certain instances, the RNA molecule is an antisense molecule, optionally, an siRNA, shRNA, or miRNA, which targets a protein of an immune checkpoint pathway for reduced expression. In various aspects, the protein of the immune checkpoint pathway is CTLA-4, PD-1, PD-L1, PD-L2, B7-H3, B7-H4, TIGIT, LAG3, CD112 TIM3, BTLA, or co-stimulatory receptor ICOS, OX40, 41BB, or GITR. The protein of the immune-checkpoint pathway in certain instances is CTLA4, PD-1, PD-L1, B7-H3, B7H4, or TIM3. Immune checkpoint signaling pathways are reviewed in Pardoll, Nature Rev Cancer 12(4): 252-264 (2012).

In exemplary embodiments, the nanoparticles of the present disclosure comprise a mixture of RNA molecules. In exemplary aspects, the mixture of RNA molecules is RNA isolated from cells from a human and optionally, the human has a tumor. In some aspects, the mixture of RNA is RNA isolated from the tumor of the human. In exemplary aspects, the human has cancer, optionally, any cancer described herein. Optionally, the tumor from which RNA is isolated is selected from the group consisting of a glioma (including, but not limited to, a glioblastoma), a medulloblastoma, a diffuse intrinsic pontine glioma, or a peripheral tumor with metastatic infiltration into the central nervous system (e.g., melanoma or breast cancer). In exemplary aspects, the tumor from which RNA is isolated is a tumor of a cancer, e.g., any of these cancers described herein.

In various aspects, the nanoparticles comprise a nucleic acid molecule (e.g., RNA molecule) comprising a nucleotide sequence encoding a chimeric protein comprising a LAMP protein. In certain aspects, the LAMP protein is a LAMP1, LAMP 2, LAMP3, LAMP4, or LAMP5 protein.

In various aspects the nanoparticles of the present disclosure comprise nucleic acid molecules comprising a sequence of a nucleic acid molecule expressed by slow-cycling cells (SCCs). In various aspects, nucleic acid molecules are RNA, e.g., optionally, tRNA, rRNA, mRNA, siRNA, shRNA, or the like. In exemplary instances, the nucleic acid molecules are RNA extracted from isolated SCCs or are nucleic acid molecules which hybridize to RNA extracted from isolated SCCs.

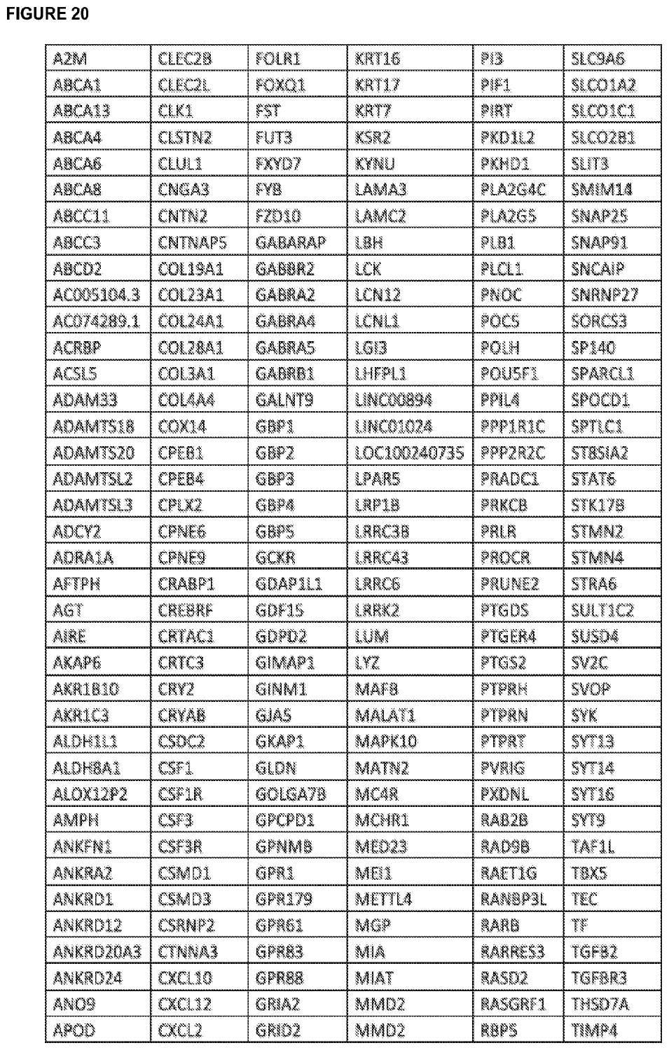

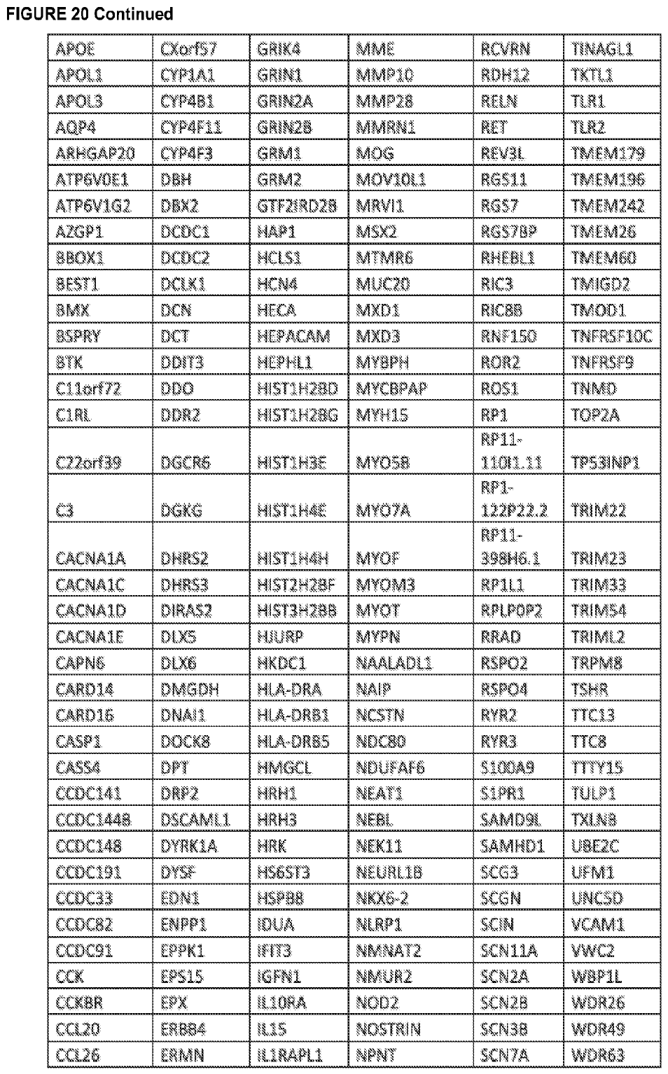

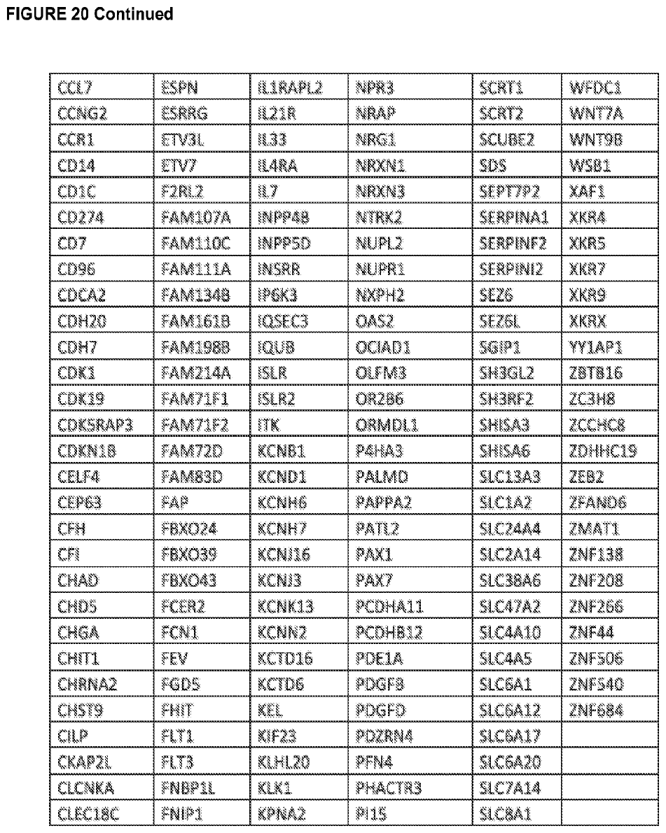

In exemplary instances, the nucleic acid molecules are encoded by at least one gene listed in FIG. 20. Optionally, the nucleic acid molecules are encoded by at least or about 2, 3, 4, 5, 6, 7, 8, 9, 10, 11, 12, 13, 14, 15, 16, 17, 18, 19, or 20 genes listed in FIG. 20. The nucleic acid molecules in various aspects are encoded by more than about 50, 60, 70, 80, 90, 100 genes listed in FIG. 20. In some instances, the nucleic acid molecules are encoded by at least or about 200, 300, 400, 500, or 600 genes listed in FIG. 20.

In exemplary instances, the nucleic acid molecules comprise mRNA expressed by SCCs. In exemplary instances, the mRNA is prepared by amplifying transcribed mRNA from cDNA libraries generated by reverse transcription from total RNA isolated from SCCs. Optionally, the SCCs are isolated from a mixed tumor cell population obtained from a subject with a tumor. In various instances, the tumor is a glioblastoma. In various aspects, the RNA is isolated from SCCs which are isolated from a mixed tumor cell population using a flow cytometer. In exemplary instances, the SCCs are isolated from a mixed tumor cell population based on proliferation rate, mitochondrial content, lipid content or a combination thereof. In exemplary aspects, the SCCs are isolated from a mixed tumor cell population based on proliferation rate using a dye that covalently binds to free amines of intracellular proteins. Optionally, the dye is a carboxyfluorescein succinimidyl ester (CFSE) dye, a Carboxyfluorescein diacetate (CFDA) dye, a Carboxyfluorescein diacetate succinimidyl ester (CFDA-SE) dye, a Celllrace™ Proliferation dye (e.g., a CellTrace™ Violet (CTV) dye), a CellVue® Claret dye, a PKH26 dye, or an e-Fluor™ Proliferation dye. In exemplary aspects, the SCCs are isolated from a mixed tumor cell population based on mitochondrial content using a dye that binds to thiol groups in the mitochondria. Optionally, the dye comprises a thiol-reactive moiety, optionally, a thiol-reactive chloromethyl moiety. In exemplary aspects, the SCCs are isolated from a mixed tumor cell population based on lipid content using a dye that stains lipid droplets. Optionally, the dye is LipidTox or LipidSpot dye.

In exemplary instances, the nanoparticle comprises a mixture or plurality of different RNA molecules expressed by SCCs. In certain instances, the mixture or plurality comprises at least 10 (e.g., at least 20, at least 30, at least 40, at least 50, at least 60, at least 70, at least 80, at least 90) different RNA molecules expressed by SCCs. In some aspects, the mixture or plurality comprises at least 100 (e.g., at least 150, at least 200, at least 250, at least 300, at least 350, at least 400, at least 450, at least 500, at least 550, at least 600, or more (e.g., at least 700, at least 800 at least 900)) different RNA molecules expressed by SCCs. In aspects, the liposome comprises a mixture or plurality of RNA molecules which represent at least in part the transcriptome of SCCs. The term “transcriptome” refers to the sum total of all the messenger RNA molecules expressed from the genes of an organism. The term “SCC transcriptome” refers to the sum total of all the mRNA molecules expressed by SCCs. In particular instances, the SCC transcriptome is produced by first isolating total RNA from the tumor cells, which total RNA is then used to generate cDNA by RT-PCR using routine methods. The cDNA may be used to synthesize protected mRNA transcripts (e.g., 7-methyl guanosine capped RNA) using, for example, an Ambion® mMESSAGE mMACHINE® transcription kit. In exemplary aspects, the SCC transcriptome is the sum total of all the mRNA expressed from the genes listed in Supplementary Table 1. In alternative or additional embodiments, the nucleic acid molecules of the liposomes, e.g., the RNA, are de novo synthesized RNA encoded by at least two (e.g., at least 3, at least 4, at least 5, at least 6, at least 7, at least 8, at least 9) different genes listed in FIG. 20. In exemplary instances, the nucleic acid molecules are RNA encoded by at least 10 (e.g., at least 20, at least 30, at least 40, at least 50, at least 60, at least 70, at least 80, at least 90) different genes listed in FIG. 20. In some aspects, the nucleic acid molecules are RNA encoded by at least 100 (e.g., at least 150, at least 200, at least 250, at least 300, at least 350, at least 400, at least 450, at least 500, at least 550, at least 600, or more (e.g., at least 700, at least 800 at least 900)) different genes listed FIG. 20.

In exemplary aspects, the nucleic acid molecules of the liposomes, e.g., the RNA, are prepared by isolating total RNA from SCCs, creating a cDNA library from the total RNA, preparing (e.g., via transcription) mRNA from the cDNA library, and amplifying the mRNA. In exemplary aspects, the SCCs from which total RNA is isolated are SCCs isolated from a sample obtained from a subject, e.g., a human. In exemplary aspects, the subject from whom the sample is obtained is the same subject to be treated with the anti-tumor liposome composition. In some aspects, the subject has a tumor or cancer, optionally, any cancer or tumor described herein. Optionally, the tumor is selected from the group consisting of: a glioma (including, but not limited to, a glioblastoma), a medulloblastoma, a diffuse intrinsic pontine glioma, or a peripheral tumor with metastatic infiltration into the central nervous system (e.g., melanoma or breast cancer).

In exemplary aspects, the nucleic acid molecules are complexed with the cationic lipid via electrostatic interactions. In exemplary aspects, the anti-tumor liposomes are prepared by mixing the RNA expressed by SCCs and the cationic lipid at a RNA: cationic lipid ratio of about 1 to about 10 to about 1 to about 25. In exemplary aspects, the anti-tumor liposomes are prepared by mixing the RNA expressed by SCCs and the cationic lipid at a RNA: cationic lipid ratio of about 1 to about 10 to about 1 to about 20 (e.g., about 1 to about 19, about 1 to about 18, about 1 to about 17, about 1 to about 16, about 1 to about 15, about 1 to about 14, about 1 to about 13, about 1 to about 12, about 1 to about 11). In exemplary instances, the liposomes are prepared by mixing RNA and the cationic lipid at a RNA: cationic lipid ratio of about 1 to about 15. Methods of complexing nucleic acid molecules, e.g., RNA, with cationic lipids for purposes of making liposomes or nanoparticles are described in the art. See, e.g., Sayour et al., Oncoimmunology 6(1): e1256527 (2017).

Methods of Manufacture

The present disclosure also provides a method of making a nanoparticle comprising a positively-charged surface and an interior comprising (i) a core and (ii) at least two nucleic acid (e.g., RNA) layers, wherein each nucleic acid layer is positioned between a cationic lipid bilayer, said method comprising: (A) mixing nucleic acid molecules and liposomes at a nucleic acid: liposome ratio of about 1 to about 5 to about 1 to about 25, such as about 1 to about 5 to about 1 to about 20, optionally, about 1 to about 15, to obtain a nucleic acid-coated liposomes. The liposomes are made by a process comprising drying a lipid mixture comprising a cationic lipid and an organic solvent by evaporating the organic solvent under a vacuum. The method further comprises (B) mixing the nucleic acid-coated liposomes with a surplus amount of liposomes. In some aspects of the disclosure, the nucleic acid is RNA and binds to or encodes an epitope of a nucleic acid encoding a fusion protein expressed by a tumor. In other aspects of the disclosure, comprises nucleic acid molecules comprising a sequence of a nucleic acid molecule expressed by slow-cycling cells (SCCs).

In various aspects, the nucleic acid molecules are RNA. Optionally, the nucleic acid molecules are extracted from isolated SCCs. In some embodiments, the method further comprises extracting RNA from the isolated SCCs. Optionally, the method further comprises preparing mRNA by amplifying transcribed mRNA from cDNA libraries generated by reverse transcription from total RNA isolated from SCCs. In various instances, the method further comprises isolating SCCs from a mixed tumor cell population, optionally, by using a flow cytometer. Methods of isolating SCCs are known in the art and include for example those described herein and in Hoang-Minh et al., 2018, supra. In exemplary aspects, the method comprises isolating SCCs from a mixed tumor cell population based on proliferation rate, mitochondrial content, lipid content or a combination thereof. In various instances, the method comprises isolating the SCCs from a mixed tumor cell population based on proliferation rate using a dye that covalently binds to free amines of intracellular proteins, optionally, wherein the dye is a carboxyfluorescein succinimidyl ester (CFSE) dye, a Carboxyfluorescein diacetate (CFDA) dye, a Carboxyfluorescein diacetate succinimidyl ester (CFDA-SE) dye, a CellTrace™ Proliferation dye (e.g., a CellTrace™ Violet (CTV) dye), a CellVue® Claret dye, a PKH26 dye, or an e-Fluor™ Proliferation dye. In various instances, the method comprises isolating the SCCs from a mixed tumor cell population based on mitochondrial content using a dye that binds to thiol groups in the mitochondria, optionally, wherein the dye comprises a thiol-reactive moiety, optionally, a thiol-reactive chloromethyl moiety. In various aspects, the method comprises isolating the SCCs from a mixed tumor cell population based on lipid content using a dye that stains lipid droplets, optionally, wherein the dye is LipidTox or LipidSpot dye. In various instances, the nucleic acid molecules are encoded by at least one gene listed in FIG. 20. In some aspects, the nucleic acid molecules are encoded by at least or about 2, 3, 4, 5, 6, 7, 8, 9, 10, 11, 12, 13, 14, 15, 16, 17, 18, 19, or 20 genes listed in FIG. 20 or are encoded by more than about 50, 60, 70, 80, 90, 100 genes listed in FIG. 20 or are encoded by at least or about 200, 300, 400, 500, or 600 genes listed in FIG. 20.

In exemplary aspects, the nanoparticle made by the presently disclosed method accords with the descriptions of the presently disclosed nanoparticles described herein. For example, the nanoparticle made by the presently disclosed methods has a zeta potential of about +40 mV to about +60 mV, optionally, about +45 mV to about +55 mV. Optionally, the zeta potential of the nanoparticle made by the presently disclosed methods is about +50 mV. In various aspects, the core of the nanoparticle made by the presently disclosed methods comprises less than about 0.5 wt % nucleic acid and/or the core comprises a cationic lipid bilayer and/or the outermost layer of the nanoparticle comprises a cationic lipid bilayer and/or the surface of the nanoparticle comprises a plurality of hydrophilic moieties of the cationic lipid of the cationic lipid bilayer.

In exemplary aspects, the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent. In various instances, the process of making liposomes further comprises rehydrating the lipid mixture with a rehydration solution to form a rehydrated lipid mixture and then agitating, resting, and sizing the rehydrated lipid mixture. Optionally, sizing the rehydrated lipid mixture comprises sonicating, extruding and/or filtering the rehydrated lipid mixture.

A description of an exemplary method of making a nanoparticle comprising a positively-charged surface and an interior comprising (i) a core and (ii) at least two nucleic acid layers, wherein each nucleic acid layer is positioned between a cationic lipid bilayer is provided herein at Example 1. Any one or more of the steps described in Example 1 may be included in the presently disclosed method. For instance, in some embodiments, the method comprises one or more steps required for preparing the RNA prior to being complexed with the liposomes. In exemplary aspects, the method comprises downstream steps to prepare the nanoparticles for administration to a subject, e.g., a human. In exemplary instances, the method comprises formulating the NP for intravenous injection. The method comprises in various aspects adding one or more pharmaceutically acceptable carriers, diluents, or excipients, and optionally comprises packaging the resulting composition in a container, e.g., a vial, a syringe, a bag, an ampoule, and the like. The container in some aspects is a ready-to-use container and optionally is for single-use.

Further provided herein are nanoparticles made by the presently disclosed method of making a nanoparticle.

Methods of Isolating SCCs

The present disclosure additionally provides an in vitro method of isolating slow-cycling cells (SCCs) from a mixed tumor cell population. As used herein, the term “mixed tumor cell population” refers to a heterogeneous cell population comprising tumor cells of different sub-types and comprising slow-cycling cells and at least one other tumor cell type, e.g., fast-cycling cells (FCCs). As used herein, the term “slow-cycling cells” or “SCCs” refers to tumor or cancer cells that proliferate at a slow rate. In exemplary aspects, the SCCs have a doubling time of at least about 50 hours. SCCs have been identified in numerous cancer tissues, including, melanoma, ovarian cancer, pancreatic adenocarcinoma, breast cancer, glioblastoma, and colon cancer. As taught in Deleyrolle et al., Brain 134(5): 1331-1343 (2011) (incorporated by reference herein, particularly with respect to the description of SCCs), SCCs display increased tumor-initiation properties and are stem cell like. Because of their slow proliferation rate, SCCs are also referred to as label-retaining cells (LRCs).

In exemplary embodiments, the method comprises (a) contacting a mixed tumor cell population with a cell proliferation dye or mitochondrial dye (e.g., MitoTracker™) which binds to cells (e.g., binds to the surface or the interior of the cells) of the mixed tumor cell population; (b) separating the dyed cells into sub-populations based on the intensity of the fluorescence emitted by the cell proliferation dye or mitochondrial dye; and (c) selecting and isolating the sub-population exhibiting the top 1-20% of fluorescence intensity or removing the sub-population exhibiting the bottom 80% of fluorescence intensity, thereby isolating SCCs from the mixed tumor cell population.

In exemplary aspects, the cell proliferation dye or mitochondrial dye comprises a thiol-reactive chloromethyl group or amine-reactive group. Optionally, the cell proliferation dye binds to the cell interior and comprises carboxyfluorescein succinimidyl ester (CFSE), optionally, CellTrace™ CFSE, CFDA-SE, CFDA, CellTrace™ Violet, Blue, Yellow, Far Red or any wavelengths of the color spectrum. In exemplary aspects, the cell proliferation dye is a cell surface binding dye such as, e.g., CellVue Claret dyes, PKH26 and e-Fluor Proliferation dyes. In exemplary aspects, the mitochondrial dye is a cell mitotracker dye comprising Rosamine-based Mitotracker probes (Orange CMTMRos, Orange CM-H2TMRos, Red CMXRos, Red CM-H2XRos, Deep Red CMXRos, Deep Red CM-H2XRos) and Carbocyanin-based Mitotracker probes (Green FM, Orange FM, Red FM, Deep Red FM).

Additional dyes that could be used in the presently disclosed method of isolating SCCs include, but are not limited to, CellTrace Proliferation dyes (Blue, Violet, CFSE, Yellow, Far Red), CFDA, CFDA-SE, CellVue Claret dyes, PKH26 and e-Fluor Proliferation dyes). The concentration of the dyes may vary from 0.1 uM to 50 uM and the labeling time may vary from 1 minute to 1 hour. The labeling solution may be PBS or any serum-free or protein-free medium. The cell density for labeling may be from 0.1 million cells per ml of labeling solution to 20 million cells per ml of labeling solution. A chasing period may need to be performed after labeling. After this chasing period, which varies between 2 days and 8 weeks, the labeling intensity is quantified by flow cytometry.

In exemplary embodiments, the method comprises a combination of one or more of the aforementioned dyes. In some aspects, the method comprises contacting a mixed tumor cell population with at least two cell proliferation or mitochondrial dyes, optionally, at least 3, at least 4, at least 5, at least 6, or more cell proliferation or mitochondrial dyes.

The SCCs selected may be those cells exhibiting the most fluorescence. In exemplary aspects, the SCCs represent the top 1 to 20% of cells having the highest fluorescence intensity. In aspects, FCCs (fast-cycling cells) may be those cells exhibiting the least fluorescence. In exemplary aspects, the FCCs represent the bottom 1 to 20% cells having the lowest fluorescence intensity. Accordingly, to isolate SCCs, the method in exemplary instances comprises selecting and isolating the sub-population of cells exhibiting the top 1-20% of fluorescence intensity. In exemplary aspects, the method comprises selecting and isolating the sub-population of cells exhibiting the top 1%, top 2%, top 3%, top 4%, top 5%, top 6%, top 7%, top 8%, top 9%, top 10%, top 11%, top 12%, top 13%, top 14%, top 15%, top 16%, top 17%, top 18%, top 19% or the top 20% fluorescence intensity. The selection of cells based on fluorescence intensity may be achieved through techniques of flow cytometry and cell sorting, e.g., fluorescence-activated cell sorting (FACS). It is understood that larger isolated fractions may work with less efficacy and smaller fractions may work with less efficiency. SCCs and FCCs are identified based on their respective ability to be stained and retain labeling.

In exemplary aspects, the method comprises removing dead cells from the mixed tumor cell population. In some aspects, the method comprises contacting the cells of the mixed tumor cell population with a dead cell stain agent including but not limited to propidium iodide (PI), non-fixable SYTOX DNA-binding dyes (e.g., SYTOX AADvanced, SYTOX Blue, SYTOX Orange, SYTOX Red or SYTOX Green) and live/dead fixable dyes (e.g., LIVE/DEAD Fixable Dead Cell Stain Blue, Aqua, Yellow, Green, Red, Far Red, Near-IR). Dead cell stain agents are dyes that enters dead cells and cannot penetrate live cells.

In exemplary aspects, the isolation of SCCs from the mixed tumor population is carried out in one of the following ways. In a first method, SCCs are isolated from the mixed population of tumor cells based on proliferation rates, as described in the Examples provided herein. In exemplary aspects, SCCs are isolated based on their capacity to retain CellTrace dyes (Carboxyfluorescein succinimidyl ester-CFSE or Cell Trace Violet-CTV, Invitrogen). The SCCs and FCCs are grouped as CFSE/Violet^{high}—top 10% and CFSE/Violet_{low}—bottom 10%, respectively, or FCCs in some aspects are isolated as CFSE_{low}—bottom 85% (Deleyrolle L P, et al. (2011) Brain 134:1331-43). Thus, SCCs in some aspects are isolated by selecting for cells grouped as CFSE/Violet^{high}—top 10% or by removing CFSE^{low}—bottom 85% (FCCs). In a second method, SCCs are isolated based on mitochondrial content. In various instances, the cell-permeant MitoTracker™ (ThermoFisher Scientific, Waltham, Mass.) probes containing a mildly thiol-reactive chloromethyl moiety for labeling mitochondria is used to alternatively identify and isolate SCCs. In alternative or additional aspects, the following dyes are used to label live cells: Rosamine-based MitoTracker dyes, which include MitoTracker Orange CMTMRos, a derivative of tetramethylrosamine, and MitoTracker Red CMXRos, a derivative of X-rosamine. Reduced MitoTracker dyes, MitoTracker Orange CM-H2TMRos and MitoTracker Red CM-H2XRos, which are derivatives of dihydrotetramethylrosamine and dihydro-X-rosamine, respectively also are used in various instances. The carbocyanine-based MitoTracker dyes including MitoTracker Red FM, MitoTracker Green FM dye, and MitoTracker® Deep Red FM are additional dyes that are suitable for use to stain mitochondria and identify SCCs. The MitoProbe™ DilC1(5) (1,1′,3,3,3′,3′-hexamethylindodicarbo-cyanine iodide), which penetrates the cytosol of eukaryotic cells and accumulates primarily in mitochondria with active membrane potentials at concentrations below 100 nM (e.g., below 90 nM, below 80 nM, below 70 nM, below 60 nM, below 50 nM, below 25 nM, below 10 nM, below 5 nM), can be used to identify and isolate SCCs, which demonstrated greater mitochondrial membrane potential. Labeling of the cells is performed at 1 nM to 100 nM for 5 minutes to 12 h (optionally, about 10 minutes to about 12 h, about 15 minutes to about 12 h, about 30 minutes to about 12 h, about 45 minutes to about 12 h, about 1 h to about 12 h, about 2 h to about 12 h, about 3 h to about 12 h, about 4 h to about 12 h, about 5 h to about 12 h, about 6 h to about 12 h, about 8 h to about 12 h). SCCs can then be identified by the up to top 50% most brightest cells. In a third method, SCCs are isolated based on lipid content. In exemplary aspects, LipidSpot is used. Live or fixed cells are incubated with LipidSpot dyes, including but not limited to LipidSpot 610 and LipidSpot 488. In other exemplary aspects, LipidTox is used. Fixed cells are incubated with lipidTox dyes including but not limited to LipidTOX Green neutral lipid stain, LipidTOX Red neutral lipid stain or LipidTOX Deep Red neutral lipid stain.

The dilutions of the dyes may vary from 1/10 to 1/5000 (e.g., about 1/10, about 1/50, about 1/100, about 1/250, about 1/500, about 1/750, about 1/1000, about 1/2000, about 1/3000, about 1/4000, about 1/5000).

The concentrations of the dyes in certain aspects range from about 5 nM to 1000 nM, e.g., about 50 nM to about 1000 nM, about 100 nM to about 1000 nM, about 150 nM to about 1000 nM, about 200 nM to about 1000 nM, about 250 nM to about 1000 nM, about 300 nM to about 1000 nM, about 350 nM to about 1000 nM, about 400 nM to about 1000 nM, about 450 nM to about 1000 nM, about 500 nM to about 1000 nM, about 550 nM to about 1000 nM, about 600 nM to about 1000 nM, about 650 nM to about 1000 nM, about 700 nM to about 1000 nM, about 750 nM to about 1000 nM, about 800 nM to about 1000 nM, about 850 nM to about 1000 nM, about 900 nM to about 1000 nM, about 950 nM to about 1000 nM, about 5 nM to about 950 nM, about 5 nM to about 850 nM, about 5 nM to about 800 nM, about 5 nM to about 750 nM, about 5 nM to about 700 nM, about 5 nM to about 650 nM, about 5 nM to about 600 nM, about 5 nM to about 550 nM, about 5 nM to about 500 nM, about 5 nM to about 450 nM, about 5 nM to about 400 nM, about 5 nM to about 350 nM, about 5 nM to about 300 nM, about 5 nM to about 250 nM, about 5 nM to about 200 nM, about 5 nM to about 150 nM, about 5 nM to about 100 nM, or about 5 nM to about 50 nM.

In various aspects, the labeling time ranges from about 1 minute to about 24 hours, about 5 minute to about 24 hours, about 10 minutes to about 24 hours, about 15 minutes to about 24 hours, about 30 minutes to about 24 hours, about 45 minutes to about 24 hours, about 1 hour to about 24 hours, about 2 hours to about 24 hours, about 3 hours to about 24 hours, about 4 hours to about 24 hours, about 5 hours to about 24 hours, about 6 hours to about 24 hours, or about 12 hours to about 24 hours.

The labeling solution may comprise PBS or any buffer. Optionally, the buffer does not comprise a detergent. In various aspects, the buffer is at a neutral pH.

The cell density for labeling may be from about 0.1 million cells per ml of labeling solution to about 20 million cells per ml of labeling solution. Optionally, the cell density is about 0.5×10⁶ to about 20×10⁶ cells per mL labeling solution, about 1×10⁶ to about 20×10⁶ cells per mL labeling solution about 2.0×10⁶ to about 20×10⁶ cells per mL labeling solution about 3.0×10⁶ to about 20×10⁶ cells per mL labeling solution about 4.0×10⁶ to about 20×10⁶ cells per mL labeling solution about 5.0×10⁶ to about 20×10⁶ cells per mL labeling solution about 6.0×10⁶ to about 20×10⁶ cells per mL labeling solution about 7.0×10⁶ to about 20×10⁶ cells per mL labeling solution about 8.0×10⁶ to about 20×10⁶ cells per mL labeling solution about 9.0×10⁶ to about 20×10⁶ cells per mL labeling solution, about 10×10⁶ to about 20×10⁶ cells per mL labeling solution, about 12.5×10⁶ to about 20×10⁶ cells per mL labeling solution, about 15×10⁶ to about 20×10⁶ cells per mL labeling solution, or about 17.5×10⁶ to about 20×10⁶ cells per mL labeling solution.

Cells and Populations Thereof

Additionally provided herein is a cell (e.g., an isolated cell or an ex vivo cell) comprising (e.g., transfected with) a nanoparticle of the present disclosure. In exemplary aspects, the cell is any type of cell that can contain the presently disclosed nanoparticle. The cell in some aspects is a eukaryotic cell, e.g., plant, animal, fungi, or algae. In alternative aspects, the cell is a prokaryotic cell, e.g., bacteria or protozoa. In exemplary aspects, the cell is a cultured cell. In alternative aspects, the cell is a primary cell, i.e., isolated directly from an organism (e.g., a human). The cell may be an adherent cell or a suspended cell, i.e., a cell that grows in suspension. The cell in exemplary aspects is a mammalian cell. Most preferably, the cell is a human cell. The cell can be of any cell type, can originate from any type of tissue, and can be of any developmental stage. In exemplary aspects, the cell comprising the liposome is an antigen presenting cell (APC). As used herein, “antigen presenting cell” or “ARC” refers to an immune cell that mediates the cellular immune response by processing and presenting antigens for recognition by certain T cells. In exemplary aspects, the APC is a dendritic cell, macrophage, Langerhans cell, or a B cell. In exemplary aspects, the APC is a dendritic cell (DC). In exemplary aspects, when the cells are administered to a subject, e.g., a human, the cells are autologous to the subject. In exemplary instances, the immune cell is a tumor associated macrophage (TAM).

Also provided by the present disclosure is a population of cells wherein at least 50%, at least 60%, at least 70%, at least 80%, at least 90%, or at least 95% of the population are cells comprising (e.g., transfected with) a nanoparticle of the present disclosure. The population of cells in some aspects is a heterogeneous cell population or, alternatively, in some aspects, is a substantially homogeneous population, in which the population comprises mainly cells comprising a nanoparticle of the present disclosure.

Pharmaceutical Compositions

Provided herein are compositions comprising a nanoparticle of the present disclosure, a cell comprising the nanoparticle of the present disclosure, a population of cells of the present disclosure, or any combination thereof, and a pharmaceutically acceptable carrier, excipient or diluent. In exemplary aspects, the composition is a pharmaceutical composition comprising a plurality of nanoparticles according to the present disclosure and a pharmaceutically acceptable carrier, diluent, or excipient and intended for administration to a human. In exemplary aspects, the composition is a sterile composition. In exemplary instances, the composition comprises a plurality of nanoparticles of the present disclosure. Optionally, at least 50% of the nanoparticles of the plurality have a diameter between about 100 nm to about 250 nm. In various aspects, the composition comprises about 10¹⁰ nanoparticles per mL to about 10¹⁵ nanoparticles per mL, optionally about 10¹² nanoparticles±10% per mL.

In exemplary aspects, the composition of the present disclosure may comprise additional components other than the nanoparticle, cell comprising the nanoparticle, or population of cells. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The composition of the present disclosure can be suitable for administration by any acceptable route, including parenteral and subcutaneous routes. Other routes include intravenous, intradermal, intramuscular, intraperitoneal, intranodal and intrasplenic, for example. In exemplary aspects, when the composition comprises the liposomes (not cells comprising the liposomes), the composition is suitable for systemic (e.g., intravenous) administration.

If the composition is in a form intended for administration to a subject, it can be made to be isotonic with the intended site of administration. For example, if the solution is in a form intended for administration parenterally, it can be isotonic with blood. The composition typically is sterile. In certain embodiments, this may be accomplished by filtration through sterile filtration membranes. In certain embodiments, parenteral compositions generally are placed into a container having a sterile access port, for example, an intravenous solution bag, or vial having a stopper pierceable by a hypodermic injection needle, or a prefilled syringe. In certain embodiments, the composition may be stored either in a ready-to-use form or in a form (e.g., lyophilized) that is reconstituted or diluted prior to administration.

Use

Without being bound to any particular theory, the data provided herein support the use of the presently disclosed RNA NPs for increasing an immune response against a tumor in a subject. Accordingly, a method of increasing an immune response against a tumor in a subject is provided by the present disclosure. In exemplary embodiments, the method comprises administering to the subject the nanoparticle or pharmaceutical composition of the present disclosure. In exemplary aspects, the nucleic acid molecules are mRNA. Optionally, the composition is systemically administered to the subject. For example, the composition is administered intravenously. In various aspects, the pharmaceutical composition is administered in an amount which is effective to activate dendritic cells (DCs) in the subject. In various instances, the immune response is a T cell-mediated immune response. Optionally, the T cell-mediated immune response comprises activity by tumor infiltrating lymphocytes (TILs). In exemplary aspects, the immune response is the innate immune response.

Also the data provided herein support the use of the presently disclosed RNA NPs for increasing dendritic cell (DC) activation in a subject. A method of activating DCs or increasing DC activation in a subject is accordingly furthermore provided. In exemplary embodiments, the method comprises administering to the subject the pharmaceutical composition of the present disclosure. In exemplary aspects, the nucleic acid molecules are mRNA. Optionally, the composition is systemically administered to the subject. For example, the composition is administered intravenously. In various aspects, the pharmaceutical composition is administered in an amount which is effective to increase an immune response against a tumor in the subject. In various instances, the immune response is a T cell-mediated immune response. Optionally, the T cell-mediated immune response comprises activity by tumor infiltrating lymphocytes (TILs). In exemplary aspects, the immune response is the innate immune response.

As used herein, the term “increase” and words stemming therefrom may not be a 100% or complete increase. Rather, there are varying degrees of increasing of which one of ordinary skill in the art recognizes as having a potential benefit or therapeutic effect. In exemplary embodiments, the increase provided by the methods is at least or about a 10% increase (e.g., at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase). In various aspects, the “increase” is in reference to baseline immunity or activation in the absence of (e.g., prior to) administering the nanoparticles of the instant disclosure.

The present disclosure also provides a method of delivering RNA molecules to an intra-tumoral microenvironment, lymph node, and/or a reticuloendothelial organ. In exemplary embodiments, the method comprises administering to the subject a presently disclosed pharmaceutical composition. Optionally, the reticuloendothelial organ is a spleen or liver. Provided herein are methods of delivery RNA to cells of a tumor, e.g., a brain tumor, comprising systemically (e.g., intravenously) administering a presently disclosed composition, wherein the composition comprises the nanoparticles. Also provided herein are methods of delivering RNA to cells in a microenvironment of a tumor, optionally a brain tumor. In exemplary embodiments, the method comprises systemically (e.g., intravenously) administering a presently disclosed composition, wherein the composition comprises the nanoparticle. In some aspects, the nanoparticle comprises an siRNA targeting a protein of an immune checkpoint pathway, optionally, PDL1. In various aspects, the cells in the microenvironment are antigen-presenting cells (APCs), optionally, tumor associated macrophages. The present disclosure also provides methods of activating antigen-presenting cells in a tumor microenvironment. In exemplary embodiments, the method comprises systemically (e.g., intravenously) administering a presently disclosed composition, wherein the composition comprises the NP.

The present disclosure provides methods of delivering RNA molecules to cells. In exemplary embodiments, the method comprises incubating the cells with the nanoparticles of the present disclosure. In exemplary instances, the cells are antigen-presenting cells (APCs), optionally, dendritic cells (DCs). In various instances, the APCs (e.g., DCs) are obtained from a subject. In certain aspects, the RNA molecules are isolated from tumor cells obtained from a subject, e.g., a human. In certain aspects, the RNA molecules are antisense molecules that target a protein of interest for reduced expression. In exemplary aspects, the RNA molecules are siRNA molecules that target a protein of the immune checkpoint pathway. Suitable proteins of the immune checkpoint pathway are known in the art and also described herein. In various instances, the siRNA target PDL1.

Once RNA has been delivered to the cells, the cells may be administered to a subject for treatment of a disease. Accordingly, the present disclosure provides a method of treating a subject with a disease. In exemplary embodiments, the method comprises delivering RNA molecules to cells of the subject in accordance with the above-described method of delivering RNA molecules to cells. In some aspects, RNA molecules are delivered to the cells ex vivo and the cells are administered to the subject. Alternatively, the method comprises administering the liposome nanoparticles directly to the subject. In exemplary embodiments, the method of treating a subject with a disease comprises administering a composition of the present disclosure in an amount effective to treat the disease in the subject. In exemplary aspects, the disease is cancer, and, in some aspects, the cancer is located across the blood brain barrier and/or the subject has a tumor located in the brain. In some aspects, the tumor is a glioma, a low grade glioma or a high grade glioma, specifically a grade III astrocytoma or a glioblastoma. Alternatively, the tumor could be a medulloblastoma or a diffuse intrinsic pontine glioma. In another example, the tumor could be a metastatic infiltration from a non-CNS tumor, e.g., breast cancer, melanoma, or lung cancer. In exemplary aspects, the composition comprises the liposome nanoparticles, and optionally, the composition comprising the liposome nanoparticles are intravenously administered to the subject. In alternative aspects, the composition comprises cells transfected with the liposome. Optionally, the cells of the composition are APCs, optionally, DCs. In exemplary aspects, the composition comprising the cells comprising the liposome nanoparticle(s) is intradermally administered to the subject, optionally, wherein the composition is intradermally administered to the groin of the subject. In exemplary instances, the DCs are isolated from white blood cells (WBCs) obtained from the subject, optionally, wherein the WBCs are obtained via leukapheresis. In some aspects, the RNA molecules bind or encode a fusion protein as described herein. In some aspects, the RNA molecules are isolated from tumor cells, e.g., cells of a tumor of the subject. In some aspects, the RNA molecules are isolated from slow-cycling cells. Accordingly, a method of treating a subject with a disease is furthermore provided herein. In exemplary embodiments, the method comprises delivering RNA molecules to cells of the subject according to the presently disclosed method of delivering RNA molecules to an intra-tumoral microenvironment, lymph node, and/or a reticuloendothelial organ. In various aspects, RNA molecules are ex vivo delivered to the cells and the cells are administered to the subject. In exemplary embodiments, the method comprises administering to the subject a pharmaceutical composition of the present disclosure in an amount effective to treat the disease in the subject. In various instances, the subject has a cancer or a tumor, optionally, a malignant brain tumor, optionally, a glioblastoma, medulloblastoma, diffuse intrinsic pontine glioma, or a peripheral tumor with metastatic infiltration into the central nervous system.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a disease of the present disclosure can provide any amount or any level of treatment. Furthermore, the treatment provided by the method may include treatment of one or more conditions or symptoms or signs of the disease being treated. For instance, the treatment method of the presently disclosure may inhibit one or more symptoms of the disease. Also, the treatment provided by the methods of the present disclosure may encompass slowing the progression of the disease. The term “treat” also encompasses prophylactic treatment of the disease. Accordingly, the treatment provided by the presently disclosed method may delay the onset or reoccurrence/relapse of the disease being prophylactically treated. In exemplary aspects, the method delays the onset of the disease by 1 day, 2 days, 4 days, 6 days, 8 days, 10 days, 15 days, 30 days, two months, 4 months, 6 months, 1 year, 2 years, 4 years, or more. The prophylactic treatment encompasses reducing the risk of the disease being treated. In exemplary aspects, the method reduces the risk of the disease 2-fold, 5-fold, 10-fold, 20-fold, 50-fold, 100-fold, or more.

In certain aspects, the method of treating the disease may be regarded as a method of inhibiting the disease or a symptom thereof. As used herein, the term “inhibit” and words stemming therefrom may not be a 100% or complete inhibition or abrogation. Rather, there are varying degrees of inhibition of which one of ordinary skill in the art recognizes as having a potential benefit or therapeutic effect. The presently disclosed methods may inhibit the onset or re-occurrence of the disease or a symptom thereof to any amount or level. In exemplary embodiments, the inhibition provided by the methods is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition).

The methods of treating a subject described herein are intended to provide an improvement in a disease or condition, and/or an improvement in the symptoms associated with the disease or condition. Any improvement in the subject's well being is contemplated (e.g., at least or about a 10% reduction, at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, or at least or about a 95% reduction of any parameter described herein). For example, a therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth or appearance of new lesions; (6) decrease in tumor size or burden; (7) absence of clinically detectable disease, (8) decrease in levels of cancer markers; (9) an increased patient survival rate; and/or (10) some relief from one or more symptoms associated with the disease or condition (e.g., pain). In various aspects, the methods of the disclosure further comprise monitoring treatment in the subject. Therapeutic responses in any given disease or condition can be determined by standardized response criteria specific to that disease or condition. Tumor response can be assessed using screening techniques such as magnetic resonance imaging (MRI) scan, x-radiographic imaging, computed tomographic (CT) scan, positron emission tomography (PET) scan, bone scan, ultrasound, tumor biopsy sampling, counting of tumor cells in circulation, and/or measurement of tumor antigen.

With regard to the foregoing methods, the nanoparticles or the composition comprising the same in some aspects is systemically administered to the subject. Optionally, the method comprises administration of the liposome nanoparticles or composition by way of parenteral administration. In various instances, the liposome nanoparticles or composition is administered to the subject intravenously.

In various aspects, the nanoparticles or composition is administered according to any regimen including, for example, daily (1 time per day, 2 times per day, 3 times per day, 4 times per day, 5 times per day, 6 times per day), three times a week, twice a week, every two days, every three days, every four days, every five days, every six days, weekly, bi-weekly, every three weeks, monthly, or bi-monthly. In various aspects, the liposomes or composition is/are administered to the subject once a week.

Subjects

The subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human is a pediatric patient or a child aged 17 years or less. In exemplary aspects, the subject has a DMG. In various instances, the DMG is diffuse intrinsic pontine glioma (DIPG).

Cancer

The cancer referenced herein may be any cancer, e.g., any malignant growth or tumor caused by abnormal and uncontrolled cell division that may spread to other parts of the body through the lymphatic system or the blood stream, that is treatable by the methods disclosed. In various aspects, the cancer is one in which cancer cells express a fusion protein.

The cancer in some aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, or lung cancer, e.g., non-small cell lung cancer (NSCLC) or bronchioloalveolar carcinoma. Other cancers and tumor types treatable by the methods of the present disclosure are contemplated, some of which are described here in a different section.

Exemplary embodiments are described in the numbered paragraphs below:

1. A composition comprising a liposome comprising ribonucleic acid (RNA) molecules and a cationic lipid, wherein the RNA molecules bind to or encode an epitope of a nucleic acid encoding a fusion protein expressed by a tumor.

2. The composition of claim 1, wherein the epitope comprises a junction of the nucleic acid encoding the fusion protein.

3. The composition of claim 1 or 2, wherein the epitope encodes an amino acid sequence which binds to an MHC Class II.

4. The composition of any one of claims 1-3, wherein the tumor is a solid tumor, optionally, a refractory solid tumor.

5. The composition of any one of claims 1-4, wherein the tumor is a malignant tumor.

6. The composition of any one claims 1-5, wherein the tumor is a brain tumor.

7. The composition of any one of claims 1-6, wherein the tumor is a sarcoma.

8. The composition of any one claims 1-7, wherein the tumor is a resistant supratentorial ependymoma or a metastatic alveolar rhabdomyosarcoma.

9. The composition of any one of claims 1-8, wherein the fusion protein is a C11orf95-RELA fusion protein or a fusion protein described herein or in Parker and Zhang, Chin J Cancer 32(11): 594-603 (2013); Ding et al., In J Mol Sci 19(1): 177 (2018), Wener et al., Molecular Cancer 17, article number 28 (2018); Yu et al., Scientific Reports 9, article number 1074 (2019).

10. The composition of any one of claims 1-9, comprising a nanoparticle comprising a positively-charged surface and an interior comprising (i) a core and (ii) at least two nucleic acid layers, wherein each nucleic acid layer is positioned between a cationic lipid bilayer.

11. The composition of claim 10, wherein the nanoparticle comprises at least three nucleic acid layers, each of which is positioned between a cationic lipid bilayer.

12. The composition of claim 11, wherein the nanoparticle comprises at least four nucleic acid layers, each of which is positioned between a cationic lipid bilayer.

13. The composition of claim 12, wherein the nanoparticle comprises five or more nucleic acid layers, each of which is positioned between a cationic lipid bilayer.

14. The composition of any one of claims 10-13, wherein the outermost layer of the nanoparticle comprises a cationic lipid bilayer.

15. The composition of any one of claims 10-14, wherein the surface comprises a plurality of hydrophilic moieties of the cationic lipid bilayer.

16. The composition of any one of claims 10-15, wherein the core comprises a cationic lipid bilayer.

17. The composition of any one of claims 10-16, wherein the core comprises less than about 0.5 wt % nucleic acid.

18. The composition of any one of claims 10-17, wherein the diameter of the nanoparticle is about 50 nm to about 250 nm in diameter, optionally, about 70 nm to about 200 nm in diameter.

19. The composition of any one of claims 10-18, wherein the nanoparticle comprises a zeta potential of about 40 mV to about 60 mV, optionally, about 45 mV to about 55 mV.

20. The composition of claim 19, comprising a zeta potential of about 50 mV.

21. The composition of any one of claims 1-20, comprising nucleic acid molecules and cationic lipid at a ratio of about 1 to about 5 to about 1 to about 20, optionally, about 1 to about 15 or about 1 to about 7.5.

22. The composition of any one claims 1-21, wherein the cationic lipid is DOTAP or DOTMA.

23. The composition of any one claims 1-22, wherein the RNA molecules are mRNA.

24. The composition of claim 23, wherein the mRNA is in vitro transcribed mRNA wherein the in vitro transcription template is cDNA made from RNA extracted from a tumor cell.

25. The composition of any one of claims 1-24, wherein the liposomes are prepared by mixing the nucleic acid molecules and the cationic lipid at an RNA: cationic lipid ratio of about 1 to about 5 to about 1 to about 20, optionally, about 1 to about 15.

26. A method of making a nanoparticle comprising a positively-charged surface and an interior comprising (i) a core and (ii) at least two nucleic acid layers, wherein each nucleic acid layer is positioned between a cationic lipid bilayer, said method comprising: (A) mixing nucleic acid molecules and liposomes at a RNA: liposome ratio of about 1 to about 5 to about 1 to about 20, optionally, about 1 to about 15, to obtain a RNA-coated liposomes, wherein the liposomes are made by a process of making liposomes comprising drying a lipid mixture comprising a cationic lipid and an organic solvent by evaporating the organic solvent under a vacuum; and (B) mixing the RNA-coated liposomes with a surplus amount of liposomes, wherein the RNA binds to or encodes an epitope of a nucleic acid encoding a fusion protein expressed by a tumor.

27. The method of claim 26, wherein the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent.

28. The method of claim 26 or 27, wherein the process of making liposomes further comprises rehydrating the lipid mixture with a rehydration solution to form a rehydrated lipid mixture and then agitating, resting, and sizing the rehydrated lipid mixture.

29. The method of claim 28, wherein sizing the rehydrated lipid mixture comprises sonicating, extruding and/or filtering the rehydrated lipid mixture.

30. The method of any one of claims 26-29, comprising the steps of Example 1.

31. The method of any one of claims 26-30, wherein the nanoparticle has a zeta potential of about 40 mV to about 60 mV, optionally, about 45 mV to about 55 mV.

32. The method of any one of claims 26-31, wherein the core of the nanoparticle comprises less than about 0.5 wt % nucleic acid and/or the core comprises a cationic lipid bilayer

33. The method of any one of claims 26-32, wherein the outermost layer of the nanoparticle comprises a cationic lipid bilayer and/or the surface of the nanoparticle comprises a plurality of hydrophilic moieties of the cationic lipid bilayer.

34. A nanoparticle made by the method of any one of claims 26-33.

35. A cell comprising a nanoparticle as described in any one of claims 1-25 or according to claim 34.

36. The cell of claim 35, which is an antigen-presenting cell (APC), optionally, a dendritic cell (DC).

37. A population of cells, wherein at least 50% of the population are cells according to any one of claim 35 or 36.

38. A pharmaceutical composition comprising a plurality of nanoparticles according to any one of claims 1-25 or claim 34 and a pharmaceutically acceptable carrier, diluent, or excipient.

39. The pharmaceutical composition of claim 38, wherein the composition comprises about 10¹⁰ nanoparticles per mL to about 10¹⁵ nanoparticles per mL, optionally about 10¹² nanoparticles±10% per mL.

40. A method of increasing an immune response against a tumor in a subject, comprising administering to the subject the pharmaceutical composition of claim 38 or 39.

41. The method of claim 40, wherein the nucleic acid molecules are mRNA.

42. The method of claim 40 or 41, wherein the composition is systemically administered to the subject.

43. The method of claim 42, wherein the composition is administered intravenously.

44. The method of any one of claims 40-43, wherein the pharmaceutical composition is administered in an amount which is effective to activate dendritic cells (DCs) in the subject.

45. The method of any one of claims 40-44, wherein the immune response is a T cell-mediated immune response.

46. A method of treating a subject with a disease, comprising administering to the subject a pharmaceutical composition of claim 38 or 39 in an amount effective to treat the disease in the subject.

47. The method of claim 46, wherein the subject has cancer or a tumor.

48. The method of claim 47, wherein the pharmaceutical composition is administered intravenously to the patient.

49. The method of any one of claims 46-48, wherein the patient is a pediatric patient.

50. A nanoparticle comprising a positively-charged surface and an interior comprising (i) a core and (ii) at least two nucleic acid layers, wherein each nucleic acid layer is positioned between a cationic lipid bilayer, and nucleic acid molecules in the nucleic acid layers comprise a sequence of a nucleic acid molecule expressed by slow-cycling cells (SCCs).

51. The nanoparticle of claim 50, comprising at least three nucleic acid layers, each of which is positioned between a cationic lipid bilayer.

52. The nanoparticle of claim 51, comprising at least four nucleic acid layers, each of which is positioned between a cationic lipid bilayer.

53. The nanoparticle of claim 52, comprising five or more nucleic acid layers, each of which is positioned between a cationic lipid bilayer.

54. The nanoparticle of any one of claims 50-53, wherein the outermost layer of the nanoparticle comprises a cationic lipid bilayer.

55. The nanoparticle of any one of claims 50-54, wherein the surface comprises a plurality of hydrophilic moieties of the cationic lipid of the cationic lipid bilayer.

56. The nanoparticle of any one of claims 50-55, wherein the core comprises a cationic lipid bilayer.

57. The nanoparticle of any one of claims 50-56, wherein the core comprises less than about 0.5 wt % nucleic acid.

58. The nanoparticle of any one of claims 50-57, wherein the diameter of the nanoparticle is about 50 nm to about 250 nm in diameter, optionally, about 70 nm to about 200 nm in diameter.

59. The nanoparticle of any one of claims 50-58, comprising a zeta potential of about 40 mV to about 60 mV, optionally, about 45 mV to about 55 mV.

60. The nanoparticle of claim 59, comprising a zeta potential of about 50 mV.

61. The nanoparticle of any one claims 50-60, comprising nucleic acid molecules and cationic lipid at a ratio of about 1 to about 5 to about 1 to about 20, optionally, about 1 to about 15 or about 1 to about 7.5.

62. The nanoparticle of any one claims 50-61, wherein the cationic lipid is DOTAP or DOTMA.

63. The nanoparticle of any one claims 50-62, wherein the nucleic acid molecules are RNA molecules.

64. The nanoparticle of claim 63, wherein the RNA molecules are mRNA.

65. The nanoparticle of claim 64, wherein the mRNA is in vitro transcribed mRNA wherein the in vitro transcription template is cDNA made from RNA extracted from a tumor cell.

66. The nanoparticle of claim 64 or 65, wherein the mRNA is prepared by amplifying transcribed mRNA from cDNA libraries generated by reverse transcription from total RNA isolated from SCCs.

67. The nanoparticle of claim 66, wherein the SCCs are isolated from a mixed tumor cell population obtained from a subject with a tumor.

68. The nanoparticle of claim 67, wherein the tumor is a glioblastoma.

69. The nanoparticle of any one of claims 64-68, wherein the RNA are isolated from SCCs which are isolated from a mixed tumor cell population using a flow cytometer.

70. The nanoparticle of claim 69, wherein the SCCs are isolated from a mixed tumor cell population based on proliferation rate, mitochondrial content, lipid content or a combination thereof.

71. The nanoparticle of claim 70, wherein the SCCs are isolated from a mixed tumor cell population based on proliferation rate using a dye that covalently binds to free amines of intracellular proteins.

72. The nanoparticle of claim 71, wherein the dye is a carboxyfluorescein succinimidyl ester (CFSE) dye, a Carboxyfluorescein diacetate (CFDA) dye, a Carboxyfluorescein diacetate succinimidyl ester (CFDA-SE) dye, a CellTrace™ Proliferation dye (e.g., a CellTrace™ Violet (CTV) dye), a CellVue® Claret dye, a PKH26 dye, or an e-Fluor™ Proliferation dye.

73. The nanoparticle of claim 70, wherein the SCCs are isolated from a mixed tumor cell population based on mitochondrial content using a dye that binds to thiol groups in the mitochondria.

74. The nanoparticle of claim 73, wherein the dye comprises a thiol-reactive moiety, optionally, a thiol-reactive chloromethyl moiety.

75. The nanoparticle of claim 70, wherein the SCCs are isolated from a mixed tumor cell population based on lipid content using a dye that stains lipid droplets.

76. The nanoparticle of claim 75, wherein the dye is LipidTox or LipidSpot dye.

77. The nanoparticle of any one of claims 50-76, comprising nucleic acid molecules encoded by at least one gene listed in FIG. 20.

78. The nanoparticle of claim 77, comprising nucleic acid molecules encoded by at least or about 2, 3, 4, 5, 6, 7, 8, 9, 10, 11, 12, 13, 14, 15, 16, 17, 18, 19, or 20 genes listed in FIG. 20.

79. The nanoparticle of claim 77, comprising nucleic acid molecules encoded by more than about 50, 60, 70, 80, 90, 100 genes listed in FIG. 20.

80. The nanoparticle of claim 77, comprising nucleic acid molecules encoded by at least or about 200, 300, 400, 500, or 600 genes listed in FIG. 20.

81. The nanoparticle of any one claims 50-81, wherein the liposomes are prepared by mixing the nucleic acid molecules and the cationic lipid at a nucleic acid molecule: cationic lipid ratio of about 1 to about 5 to about 1 to about 20, optionally, about 1 to about 15.

82. A method of making a nanoparticle comprising a positively-charged surface and an interior comprising (i) a core and (ii) at least two nucleic acid layers, wherein each nucleic acid layer is positioned between a cationic lipid bilayer, wherein the nanoparticle comprises nucleic acid molecules comprising a sequence of a nucleic acid molecule expressed by slow-cycling cells (SCCs), said method comprising: (A) mixing nucleic acid molecules comprising a sequence of a nucleic acid molecule expressed by slow-cycling cells (SCCs) and liposomes made by a process of making liposomes comprising drying a lipid mixture comprising a cationic lipid and an organic solvent by evaporating the organic solvent under a vacuum, wherein the nucleic acid molecules and the liposomes are mixed at a nucleic acid: liposome ratio of about 1 to about 5 to about 1 to about 20, optionally, about 1 to about 15, to obtain nucleic acid-coated liposomes; and (B) mixing the nucleic acid-coated liposomes with a surplus amount of liposomes.

83. The method of claim 82, wherein the nucleic acid molecules are RNA.

84. The method of claim 82 or 83, wherein the nucleic acid molecules are extracted from isolated SCCs.

85. The method of any one of claims 82-84, wherein the nucleic acid molecules are encoded by at least one gene listed in FIG. 20.

86. The method of any one of claims 82-85, further comprising extracting RNA from the isolated SCCs.

87. The method of any one of claims 82-86, further comprising preparing mRNA by amplifying transcribed mRNA from cDNA libraries generated by reverse transcription from total RNA isolated from SCCs.

88. The method of any one of claims 82-87, further comprising isolating SCCs from a mixed tumor cell population using a flow cytometer.

89. The method of claim 88, comprising isolating SCCs from a mixed tumor cell population based on proliferation rate, mitochondrial content, lipid content or a combination thereof.

90. The method of claim 89, comprising isolating the SCCs from a mixed tumor cell population based on proliferation rate using a dye that covalently binds to free amines of intracellular proteins, optionally, wherein the dye is a carboxyfluorescein succinimidyl ester (CFSE) dye, a Carboxyfluorescein diacetate (CFDA) dye, a Carboxyfluorescein diacetate succinimidyl ester (CFDA-SE) dye, a CellTrace™ Proliferation dye (e.g., a CellTrace™ Violet (CTV) dye), a CellVue® Claret dye, a PKH26 dye, or an e-Fluor™ Proliferation dye.

91. The method of claim 88, comprising isolating the SCCs from a mixed tumor cell population based on mitochondrial content using a dye that binds to thiol groups in the mitochondria, optionally, wherein the dye comprises a thiol-reactive moiety, optionally, a thiol-reactive chloromethyl moiety.

92. The method of claim 88, comprising isolating the SCCs from a mixed tumor cell population based on lipid content using a dye that stains lipid droplets, optionally, wherein the dye is LipidTox or LipidSpot dye.

93. The method of any one of claims 82-92, wherein the nucleic acid molecules are encoded by at least or about 2, 3, 4, 5, 6, 7, 8, 9, 10, 11, 12, 13, 14, 15, 16, 17, 18, 19, or 20 genes listed in FIG. 20.

94. The method of claim 93, wherein the nucleic acid molecules are encoded by more than about 50, 60, 70, 80, 90, 100 genes listed in FIG. 20.

95. The method of claim 93, wherein the nucleic acid molecules are encoded by at least or about 200, 300, 400, 500, or 600 genes listed in FIG. 20.

96. The method of any one of claims 82-95, wherein the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent.

97. The method of any one of claims 82-96, wherein the process of making liposomes further comprises rehydrating the lipid mixture with a rehydration solution to form a rehydrated lipid mixture and then agitating, resting, and sizing the rehydrated lipid mixture.

98. The method of claim 97, wherein sizing the rehydrated lipid mixture comprises sonicating, extruding and/or filtering the rehydrated lipid mixture.

99. The method of any one of claims 82-98, comprising the steps of Example 1.

100. The method of any one of claims 82-99, wherein the nanoparticle has a zeta potential of about 40 mV to about 60 mV, optionally, about 45 mV to about 55 mV.

101. The method of any one of claims 82-100, wherein the core of the nanoparticle comprises less than about 0.5 wt % nucleic acid and/or the core comprises a cationic lipid bilayer

102. The method of any one of claims 82-101, wherein the outermost layer of the nanoparticle comprises a cationic lipid bilayer and/or the surface of the nanoparticle comprises a plurality of hydrophilic moieties of the cationic lipid of the cationic lipid bilayer.

103. A nanoparticle made by the method of any one of claims 82-102.

104. A cell comprising a nanoparticle as described in any one of claims 50-81 or according to claim 103.

105. The cell of claim 104, which is an antigen presenting cell (APC), optionally, a dendritic cell (DC).

106. A population of cells, wherein at least 50% of the population are cells according to any one of claim 104 or 105.

107. A pharmaceutical composition comprising a plurality of nanoparticles according to any one of claims 50-81 or claim 103 and a pharmaceutically acceptable carrier, diluent, or excipient.

108. The pharmaceutical composition of claim 107, wherein the composition comprises about 10¹⁰ nanoparticles per mL to about 10¹⁵ nanoparticles per mL, optionally about 10¹² nanoparticles±10% per mL.

109. A method of increasing an immune response against a tumor in a subject, comprising administering to the subject the pharmaceutical composition of claim 107 or 108.

110. The method of claim 109, wherein the nucleic acid molecules are mRNA.

111. The method of claim 109 or 110, wherein the composition is systemically administered to the subject.

112. The method of claim 111, wherein the composition is administered intravenously.

113. The method of any one of claims 109-112, wherein the pharmaceutical composition is administered in an amount which is effective to activate dendritic cells (DCs) in the subject.

114. The method of any one of claims 109-113, wherein the immune response is a T cell-mediated immune response.

115. The method of claim 114, wherein the T cell-mediated immune response comprises activity by tumor infiltrating lymphocytes (TILs).

116. A method of delivering RNA molecules to an intra-tumoral microenvironment, lymph node, and/or a reticuloendothelial organ, comprising administering to the subject a pharmaceutical composition of claim 107 or 108.

117. The method of claim 116, wherein the reticuloendothelial organ is a spleen or liver.

118. A method of treating a subject with a disease, comprising administering to the subject a pharmaceutical composition of claim 107 or 108 in an amount effective to treat the disease in the subject.

119. A method of treating a subject with a disease, comprising administering to the subject the cells of claim 104 in an amount effective to treat the disease in the subject.

120. The method of claim 118 or 119, wherein the subject has a cancer or a tumor.

121. The method of claim 120, wherein the tumor is a malignant brain tumor, optionally, a glioblastoma, medulloblastoma, diffuse intrinsic pontine glioma, or a peripheral tumor with metastatic infiltration into the central nervous system.

The following examples are given merely to illustrate the present invention and not in any way to limit its scope.

### EXAMPLES

### Example 1

This example describes a method of making nanoparticles of the present disclosure.

Preparation of DOTAP Liposomes

On Day 1, the following steps were carried out in the fume hood. Water was added to a rotavapor bath. Chloroform (20 mL) was poured into a sterile, glass graduated cylinder. After opening a vial containing 1 g of DOTAP, 5 mL chloroform was added to the DOTAP vial using a glass pipette. The volume of chloroform and DOTAP was then transferred into a 1-L evaporating flask. The DOTAP vial was washed by adding a second 5-mL volume of chloroform to the DOTAP vial to dissolve any remaining DOTAP in the vial and then transferring this volume of chloroform from the DOTAP vial to the evaporating flask. This washing step was repeated 2 more times until all the chloroform in the graduated cylinder was used. The evaporating flask was then placed into the Buchi rotavapor. The water bath was turned on and adjusted to 25° C. The evaporating flask was moved downward until it touched the water bath. The rotation speed of the rotavapor was adjusted to 2. The vacuum system was turned on and adjusted to 40 mbar. After 10 minutes, the vacuum system was turned off and the chloroform was collected from the collector flask. The amount of chloroform collected was measured. Once the collector flask is repositioned, the vacuum was turned on again and the contents in the evaporating flask were allowed to dry overnight until the chloroform was completely evaporated.

On Day 2, using a sterile graduated cylinder, PBS (200 mL) was added to a new, sterile 500-mL PBS bottle maintained at room temperature. A second 500-mL PBS bottle was prepared for collecting DOTAP. The Buchi Rotavapor water bath was set to 50° C. PBS (50 mL) was added into the evaporating flask using a 25-mL disposable serological pipette. The evaporating flask was positioned in the Buchi Rotavapor and moved downward until ⅓ of the flask was submerged into the water bath. The rotation speed of the rotavapor was set to 2, allowed to rotate for 10 min, and then rotation was turned off. A 50-mL volume of PBS with DOTAP from the evaporating flask was transferred to the second 500 mL PBS bottle. The steps were repeated (3-times) until the entire volume of PBS in the PBS bottle was used. The final volume of the second 500 mL PBS bottle was 400 mL. The lipid solution in the second 500 mL PBS bottle was vortexed for 30 s and then incubated at 50° C. for 1 hour. During the 1 hour incubation, the bottle was vortexed every 10 min. The second 500 mL PBS bottle was allowed to rest overnight at room temperature.

On Day 3, PBS (200 mL) was added to the second 500 mL PBS bottle containing DOTAP and PBS. The second 500 mL PBS bottle was placed into an ultrasonic bath. Water was filled in the ultrasonic bath and the second 500 mL PBS bottle was sonicated for 5 min. The extruder was washed with PBS (100 mL) and this wash step was repeated. A 0.45 μm pore filter was assembled into a filtration unit and a new (third) 500 mL PBS bottle was positioned into the output tube of the extruder. In a biological safety cabinet, the DOTAP-PBS mixture was loaded into the extruder, until about 70% of the third PBS bottle was filled. The extruder was then turned on and the DOTAP PBS mixture was added until all the mixture was run through the extruder. Subsequently, a 0.22 μm pore filter was assembled into the filtration unit and a new (third) 500 mL PBS bottle was positioned into the output tube of the extruder. The previously filtered DOTAP-PBS mixture was loaded and run again throughout. The samples comprising DOTAP lipid nanoparticles (NPs) in PBS were then stored at 4° C.

RNA Preparation—Fusion Proteins

Prior to incorporation into nanoparticles (NPs), mini-gene RNAs spanning the length of a fusion protein is synthesized. The constructs are cloned into a modified psP73 vector (Promega), which contains a poly-A tail that is 64 nucleotides long (pSP73-Sph/A64) protecting RNA from degradation. The use of the pSP73-Sph/A64 vector allows for in vitro transcription of the DNA construct due to the presence of a T7 promoter. In vitro transcription is carried out using the mMESSAGE mMACHINE T7 transcription kit (Ambion) per manufacturer's protocol.

Fusion protein identification and mRNA manufacturing is done as follows: RNA from tumor cells expressing a fusion protein is isolated using commercially available RNeasy mini kits (Qiagen) per manufacturer's instructions. RNA is analyzed by RNA seq analysis to identify fusion proteins, and, fusion protein-specific primers are designed for amplifying and extracting the fusion protein sequences. Following amplification and extraction of the fusion protein sequences, a reverse transcriptase reaction by PCR is performed on the total tumor RNA in order to generate cDNA libraries, using a SMARTScribe Reverse Transcriptase kit (Takara). Using the fusion protein specific primers designed by amplification, the fusion protein cDNA sequence is amplified using Takara Advantage 2 Polymerase mix, and the amplified cDNA is cloned into a plasmid. The sequence is sequenced by Sanger sequencing for confirmation. In order to get sufficient mRNA for use in RNA-nanoparticles, mMESAGE mMACHINE (Invitrogen) kits with T7 enzyme mix were used to perform in vitro transcription on the plasmid to obtain mRNA. The resulting mRNA was then purified with a Qiagen RNeasy Maxi kit to obtain the final mRNA product. The final mRNA product coding for the fusion protein is then complexed with DOTAP lipid NPs, as essentially described below.

RNA Preparation—Slow-Cycling Cells

Prior to incorporation into NPs, RNA was prepared in one of a few ways. Total tumor RNA was prepared by isolating total RNA (including rRNA, tRNA, mRNA) from tumor cells. In vitro transcribed mRNA was prepared by carrying out in vitro transcription reactions using cDNA templates produced by reverse transcription of total tumor RNA. Tumor antigen-specific and Non-specific RNAs were either made in-house or purchased from a vendor.

Total Tumor RNA: Total tumor-derived RNA from tumor cells (e.g., B16F0, B16F10, and KR158-luc) is isolated using commercially available RNeasy mini kits (Qiagen) based on manufacturer instructions.

In vitro transcribed mRNA: Briefly, RNA is isolated using commercially available RNeasy mini kits (Qiagen) per manufacturer's instructions and cDNA libraries were generated by RT-PCR. Using a SMARTScribe Reverse Transcriptase kit (Takara), a reverse transcriptase reaction by PCR was performed on the total tumor RNA in order to generate cDNA libraries. The resulting cDNA was then amplified using Takara Advantage 2 Polymerase mix with T7/SMART and CDS III primers, with the total number of amplification cycles determined by gel electrophoresis. Purification of the cDNA was performed using a Qiagen PCR purification kit per manufacturer's instructions. In order to isolate sufficient mRNA for use in each RNA-nanoparticle vaccine, mMESAGE mMACHINE (Invitrogen) kits with T7 enzyme mix were used to perform overnight in vitro transcription on the cDNA libraries. Housekeeping genes were assessed to ensure fidelity of transcription. The resulting mRNA was then purified with a Qiagen RNeasy Maxi kit to obtain the final mRNA product.

Tumor Antigen-Specific and Non-Specific mRNA: Plasmids comprising DNA encoding tumor antigen-specific RNA (RNA encoding, e.g., pp65, OVA) and non-specific RNA (RNA encoding, e.g., Green Fluorescent Protein (GFP), luciferase) are linearized using restriction enzymes (i.e., Spel) and purified with Qiagen PCR MiniElute kits. Linearized DNA is subsequently transcribed using the mmRNA in vitro transcription kit (Life technologies, Invitrogen) and cleaned up using RNA Maxi kits (Qiagen). In alternative methods, non-specific RNA is purchased from Trilink Biotechnologies (San Diego, Calif.).

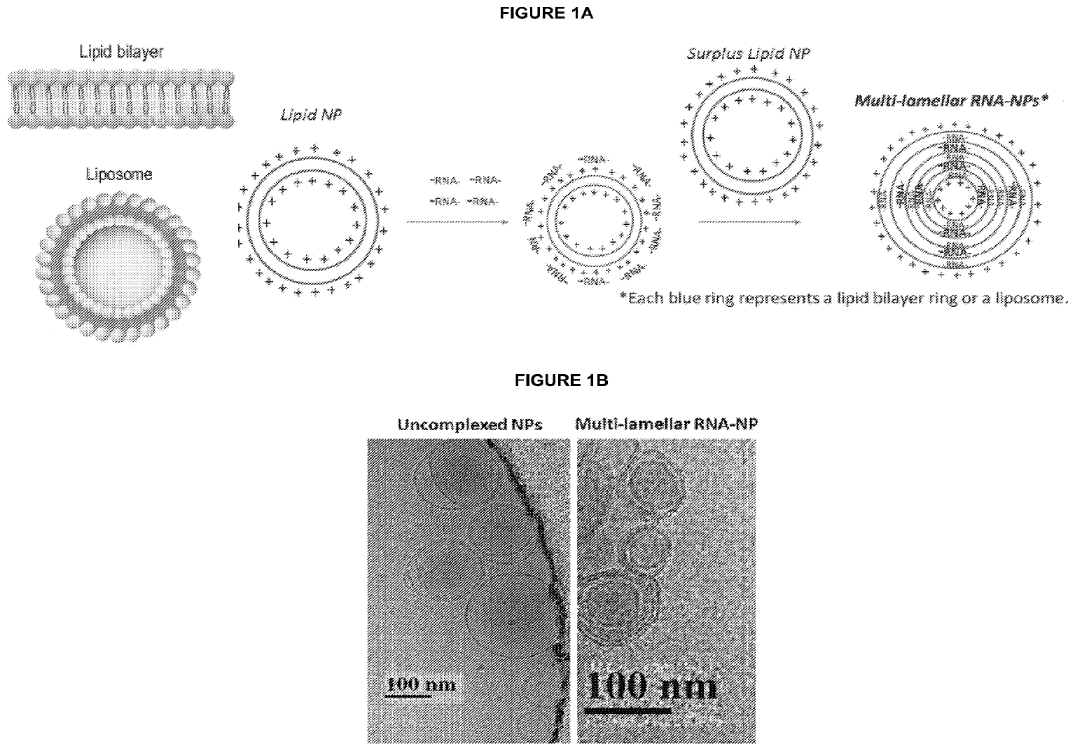

Preparation of Multilamellar RNA nanoparticles (NPs): The DOTAP lipid NPs were complexed with RNA to make multilamellar RNA-NPs which were designed to have several layers of mRNA contained inside a tightly coiled liposome with a positively charged surface and an empty core (FIG. 1A). Briefly, in a safety cabinet, RNA was thawed from −80° C. and then placed on ice, and samples comprising PBS and DOTAP (e.g., DOTAP lipid NPs) were brought up to room temperature. Once components were prepared, the desired amount of RNA was mixed with PBS in a sterile tube. To the sterile tube containing the mixture of RNA and PBS, the appropriate amount of DOTAP lipid NPs was added without any physical mixing (without e.g., inversion of the tube, without vortexing, without agitation). The mixture of RNA, PBS, and DOTAP was incubated for about 15 minutes to allow multilamellar RNA-NP formation. After 15 min, the mixture was gently mixed by repeatedly inverting the tube. The mixture was then considered ready for systemic (i.e. intravenous) administration.

The amount of RNA and DOTAP lipid NPs (liposomes) used in the above preparation is pre-determined or pre-selected. In some instances, a ratio of about 15 μg liposomes per about 1 μg RNA was used. For instance, about 75 μg liposomes are used per ˜5 μg RNA or about 375 μg liposomes are used per ˜25 μg RNA. In other instances, about 7.5 μg liposomes were used per 1 μg RNA. Thus, in exemplary instances, about 1 μg to about 20 μg liposomes are used for every μg RNA used.

### Example 2

This example describes the characterization of the nanoparticles of the present disclosure.

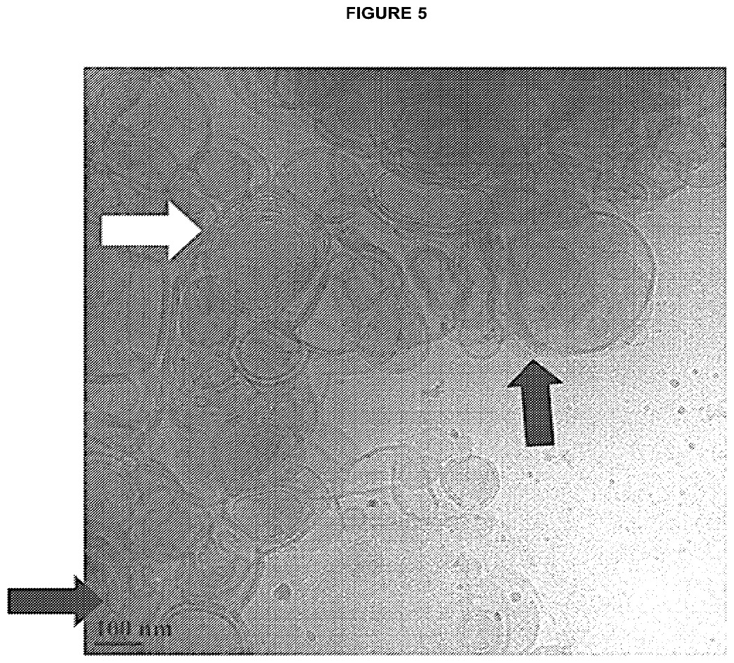

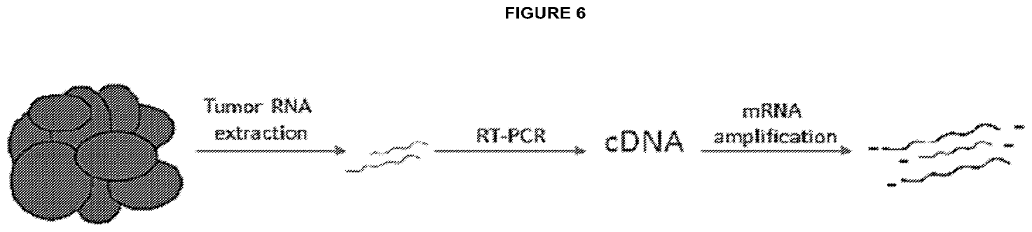

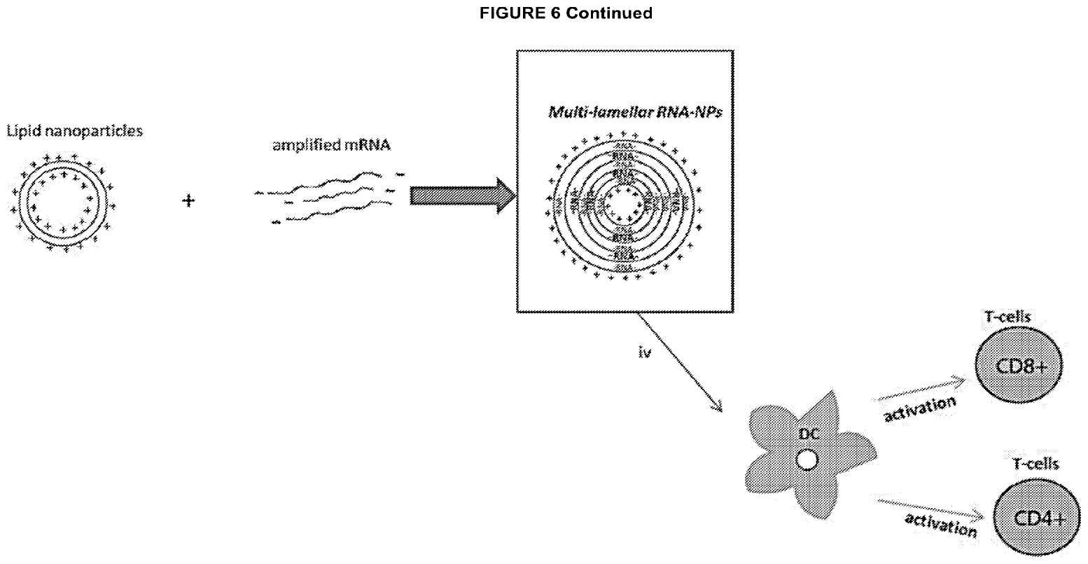

Cryo-Electron Microscopy (CEM): CEM was used to analyze the structure of multilamellar RNA-NPs prepared as described in Example 1 and control NPs devoid of RNA (uncomplexed NPs) which were made by following all the steps of Example 1, except for the steps under “RNA Preparation” and “Preparation of Multilamellar RNA nanoparticles (NPs)”. CEM was carried out as essentially described in Sayour et al., Nano Lett 17(3) 1326-1335 (2016). Briefly, samples comprising multilamellar RNA-NPs or control NPs were kept on ice prior to being loaded in a snap-freezed in Vitrobot (and automated plunge-freezer for cryoTEM, that freezes samples without ice crystal formation, by controlling temperature, relative humidity, blotting conditions and freezing velocity). Samples were then imaged in a Tecnai G2 F20 TWIN 200 kV/FEG transmission electron microscope with a Gatan UltraScan 4000 (4 k×4 k) CCD camera. The resulting CEM images are shown in FIG. 1B. The right panel is a CEM image of multilamellar RNA-NPs and the left panel is a CEM image of control NPs (uncomplexed NPs). As shown in FIG. 1B, the control NPs contained at most 2 layers, whereas multilamellar RNA NPs contained several layers. FIG. 5 provides another CEM image of exemplary multilamellar RNA NPs. Here, the multiple layers of RNA layers alternating with lipid layers are especially evident.

Zeta Potentials: Zeta potentials of multilamellar RNA NPs were measured by phase analysis light scattering (PALS) using a Brookhaven ZetaPlus instrument (Brookhaven Instruments Corporation, Holtsville, N.Y.), as essentially described in Sayour et al., Nano Lett 17(3) 1326-1335 (2016). Briefly, uncomplexed NPs or RNA-NPs (200 NL) were resuspended in PBS (1.2 mL) and loaded in the instrument. The samples were run at 5 runs per sample, 25 cycles each run, and using the Smoluchowski model.

The zeta potential of the multilamellar RNA NPs prepared as described in Example 1 was measured at about +50 mV. Interestingly, this zeta potential of the multilamellar RNA NPs was much higher than those described in Sayour et al., Oncoimmunology 6(1): e1256527 (2016), which measured at around +27 mV. Without being bound to any particular theory, the way in which the DOTAP lipid NPs are made for use in making the multilamellar RNA NPs (Example 1) involving a vacuum-seal method for evaporating off chloroform leads to less environmental oxidation of the DOTAP lipid NPs, which, in turn, may allow for a greater amount of RNA to complex with the DOTAP NPs and/or greater incorporation of RNA into the DOTAP lipid NPs.

RNA Incorporation by Gel Electrophoresis: A gel electrophoresis experiment was conducted to measure the amount of RNA incorporated into ML liposomes. Based on this experiment, it was qualitatively shown that nearly all, if not all, of the RNA used in the procedure described in Example 1 was incorporated into the DOTAP lipid NPs. Additional experiments to characterize the extent of RNA incorporation are carried out by measuring RNA-NP density and comparing this parameter to that of lipoplexes.

### Example 3

This example describes a comparison of the nanoparticles of the present disclosure to cationic RNA lipoplexes and anionic RNA lipoplexes.

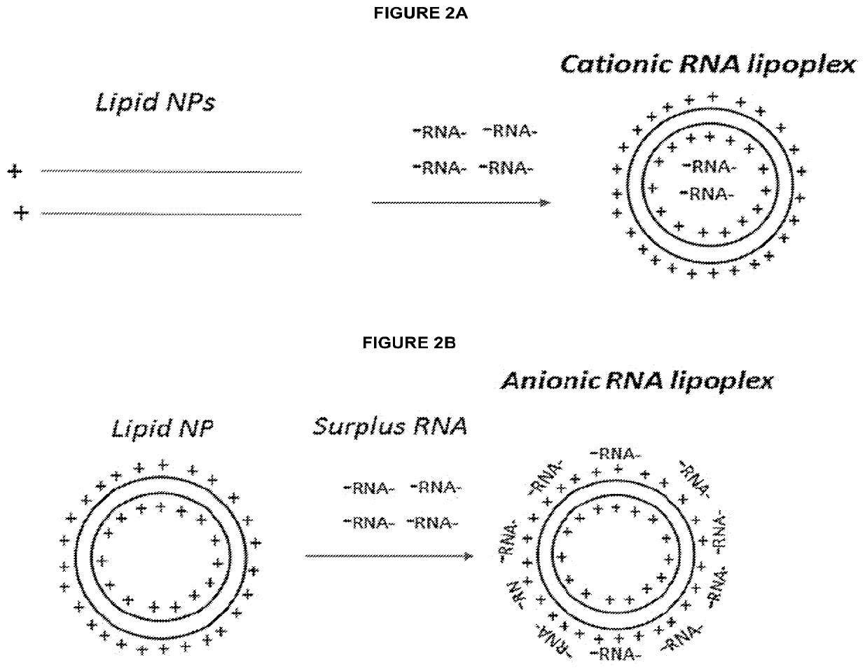

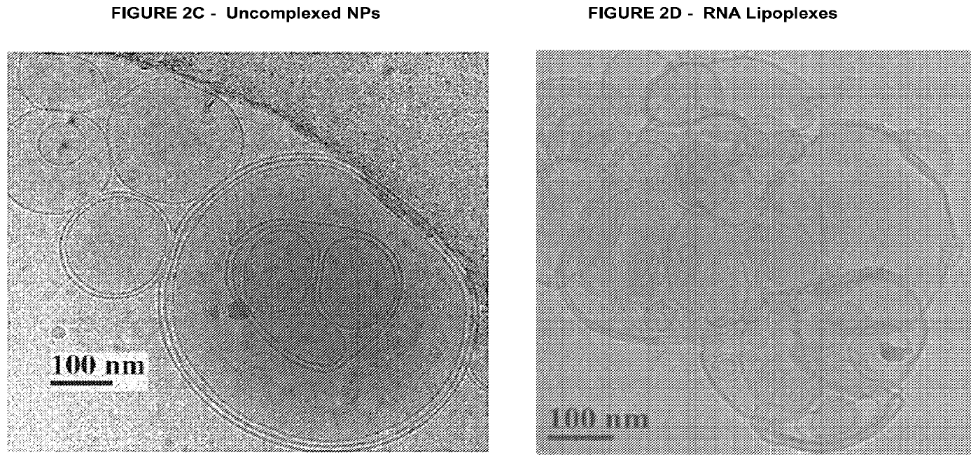

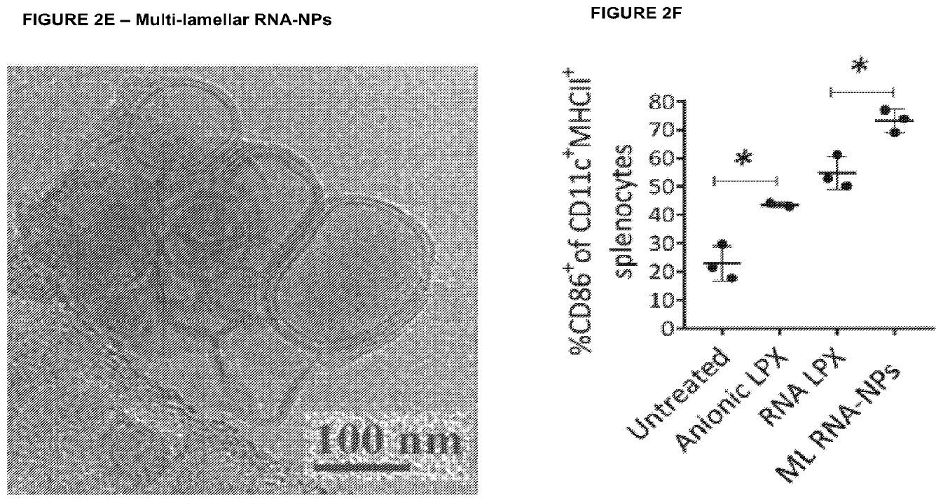

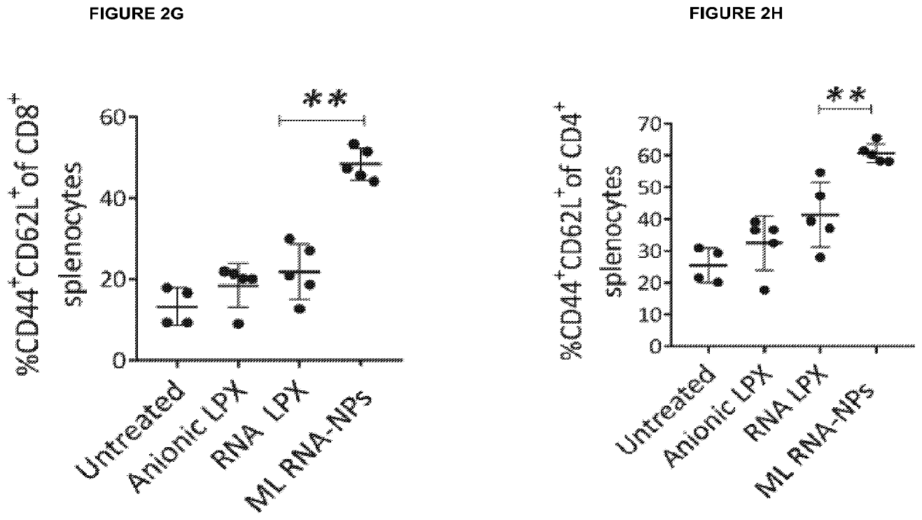

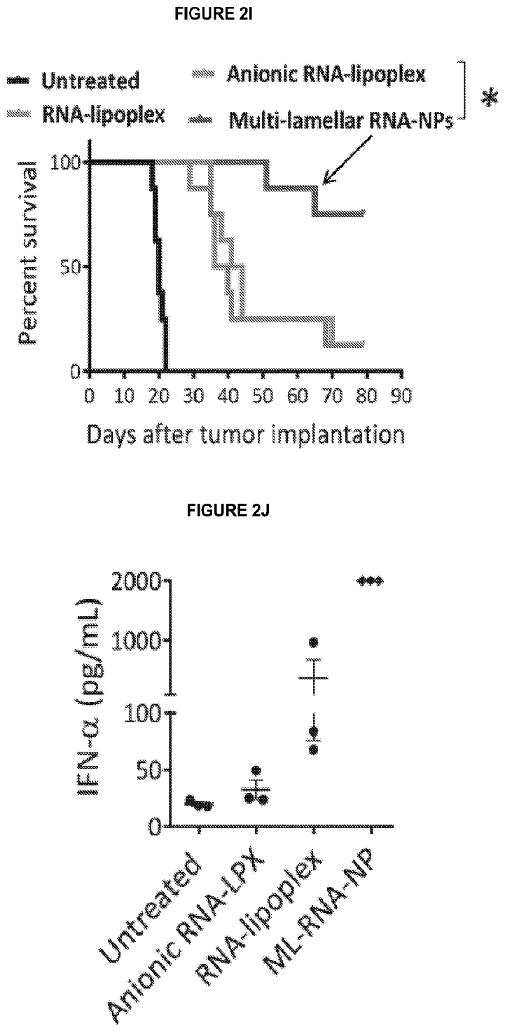

Cationic lipoplexes (LPX) were first developed with mRNA in the lipid core shielded by a net positive charge located on the outer surface (FIG. 2A). Anionic RNA lipoplexes (FIG. 2B) have been developed with an excess of RNA tethered to the surface of bi-lamellar liposomes. RNA-LPX were made by mixing RNA and lipid NP at ratios to equalize charge. Anionic RNA-NPs were made by mixing RNA and lipid NP at ratios to oversaturate lipid NPs with negative charge. Various aspects of the RNA-LPX and anionic RNA LPX were then compared to the multilamellar RNA NPs described in the above examples.

Cryo-Electron Microscopy (CEM) was used to compare the structures of the RNA LPX and the multilamellar RNA-NPs prepared as described in Example 1. Uncomplexed NPs were used as a control. CEM was carried out as essentially described in Example 2. FIG. 2C is a CEM image of uncomplexed NPs, FIG. 2D is a CEM image of RNA LPXs (wherein that mass ratio of liposome to RNA is 3.75:1), and FIG. 2E is a CEM image of the multilamellar RNA-NPs (wherein that mass ratio of liposome to RNA is 15:1). These data demonstrate that more RNA is held by the ML RNA-NPs. Additional data show that the concentration drops more with ML RNA-NP complexation versus RNA LPX supporting multilamellar formation of ML RNA-NPs not observed by simple mixing of equivalent amounts of RNA and lipid NPs by mass or charge (i.e. RNA-LPX and anionic RNA-LPX respectively). This data demonstrates that more RNA is “held” by ML RNA-NPs.

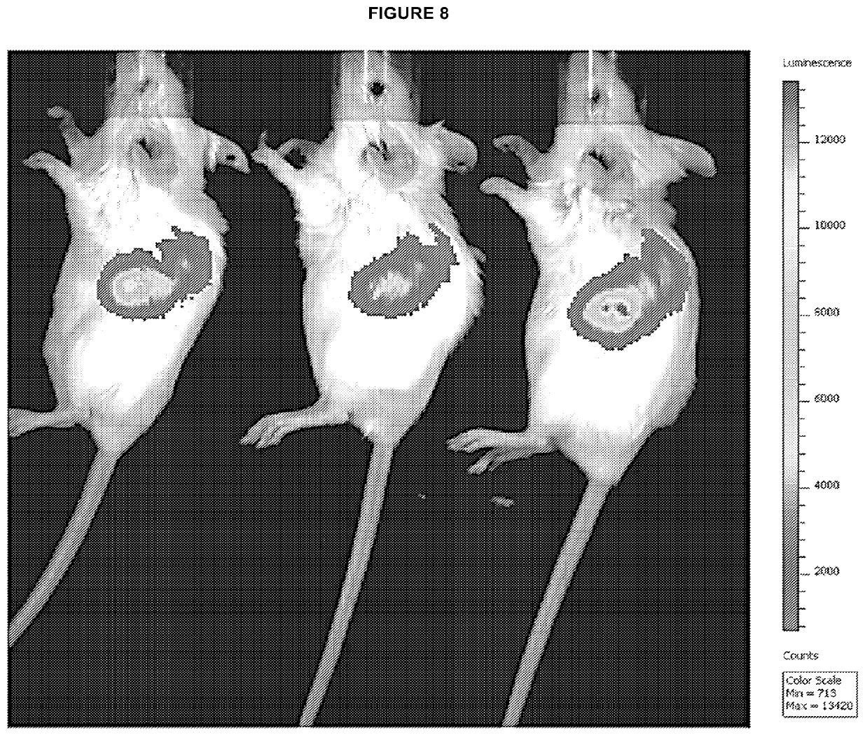

Also, an experiment was conducted to determine where the anionic LPXs localize upon administration to mice. As shown in FIG. 8, anionic LPXs localized to the spleens of animals upon administration.

RNA LPX, anionic lipoplex (LPX) or multilamellar RNA-NPs were administered to mice and spleens were harvested one week later for assessment of activated DCs (*p<0.05 unpaired t test). The RNA used in this experiment was tumor-derived mRNA from the K7M2 tumor osteosarcoma cell line. As shown in FIG. 2F, mice treated with multilamellar RNA NPs exhibited the highest levels of activated DCs.

Anionic tumor mRNA-lipoplexes, tumor mRNA-lipoplexes, and multilamellar tumor mRNA loaded NPs were compared in a therapeutic lung cancer model (K7M2) (n=5-8/group). Each vaccine was intravenously administered weekly (×3) (**p<0.01, Mann Whitney). The % CD44+CD62L+ of CD8+ splenocytes is shown in FIG. 2G and the % CD44+CD62L+ of CD4+ splenocytes is shown in FIG. 2H. Also, FIG. 2J shows that multilamellar (ML) RNA-NPs mediate substantially increased IFN-alpha which is an innate anti-viral cytokine. This demonstrates that ML RNA-NPs allow for substantially greater innate immunity which is enough to drive efficacy from even non-antigen specific ML RNA-NPs. Taken together, these figures demonstrate the superior efficacy of multilamellar tumor specific RNA-NPs, relative to anionic LPX and RNA LPX.

Anionic tumor mRNA-lipoplexes, cationic tumor mRNA-lipoplexes and multilamellar tumor mRNA loaded NPs were compared in a therapeutic lung cancer model (K7M2) (n=8/group). Each vaccine was iv administered weekly (×3), *p<0.05, Gehan Breslow-Wilcoxon test. The percent survival was measured by Kaplan-Meier Curve analysis. As shown in FIG. 2I, multilamellar tumor specific RNA-NPs mediated superior efficacy, compared to cationic RNA lipoplexes and anionic RNA lipoplexes, for increasing survival.

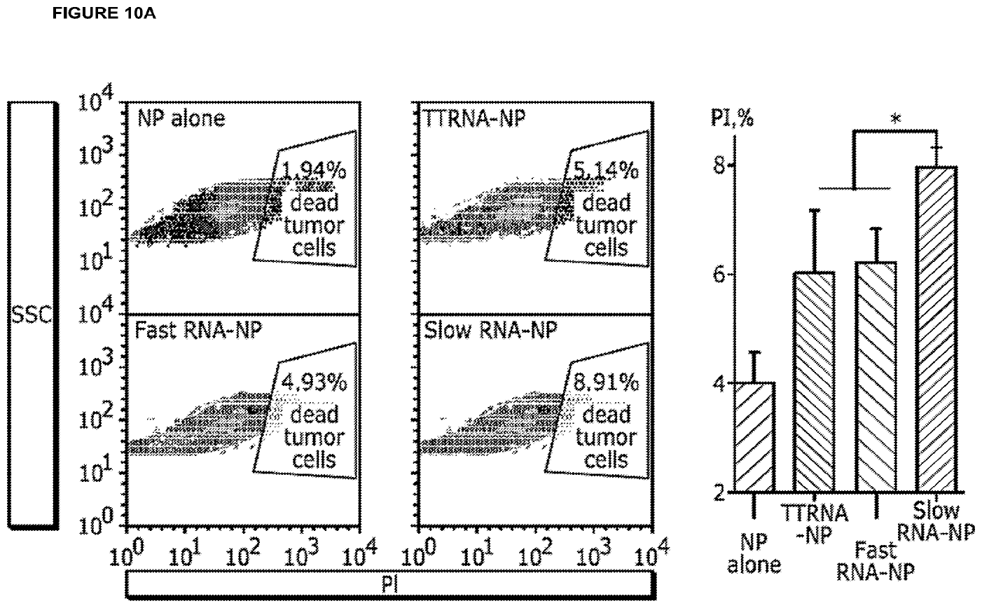

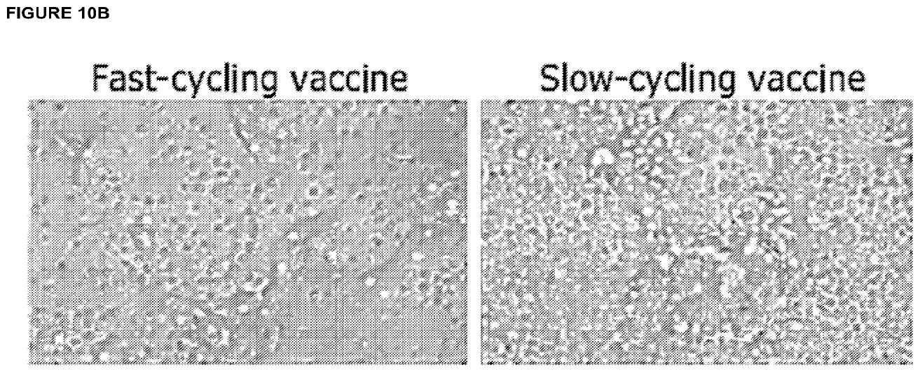

Herein it is demonstrated that the multilamellar RNA-NP formulation targeting physiologically relevant tumor antigens is more immunogenic (FIG. 2F-2H, 2J) and significantly more efficacious (FIG. 2I) compared with anionic LPX and RNA LPX. Without being bound to any particular theory, by altering RNA-lipid ratios and increasing the zeta potential, a novel RNA-NP design composed of multi-lamellar rings of tightly coiled mRNA has been developed (FIG. 10), which multi-lamellar design is thought to facilitate increased NP uptake of mRNA (condensed by alternating positive/negative charge) for enhanced particle immunogenicity and widespread in vivo localization to the periphery and tumor microenvironment (TME). Systemic administration of these multi-lamellar RNA-NPs localize to lymph nodes, reticuloendothelial organs (i.e. spleen and liver) and to the TME, activating DCs therein (based on increased expression of the activation marker CD86 on CD11c+ cells). These activated DCs prime antigen specific T cell responses, which lead to anti-tumor efficacy (with increased TILs) in several tumor models.

### Example 4

This example demonstrates the ability of multilamellar RNA-NPs to systemically activate DCs, induce antigen specific immunity and elicit anti-tumor efficacy.

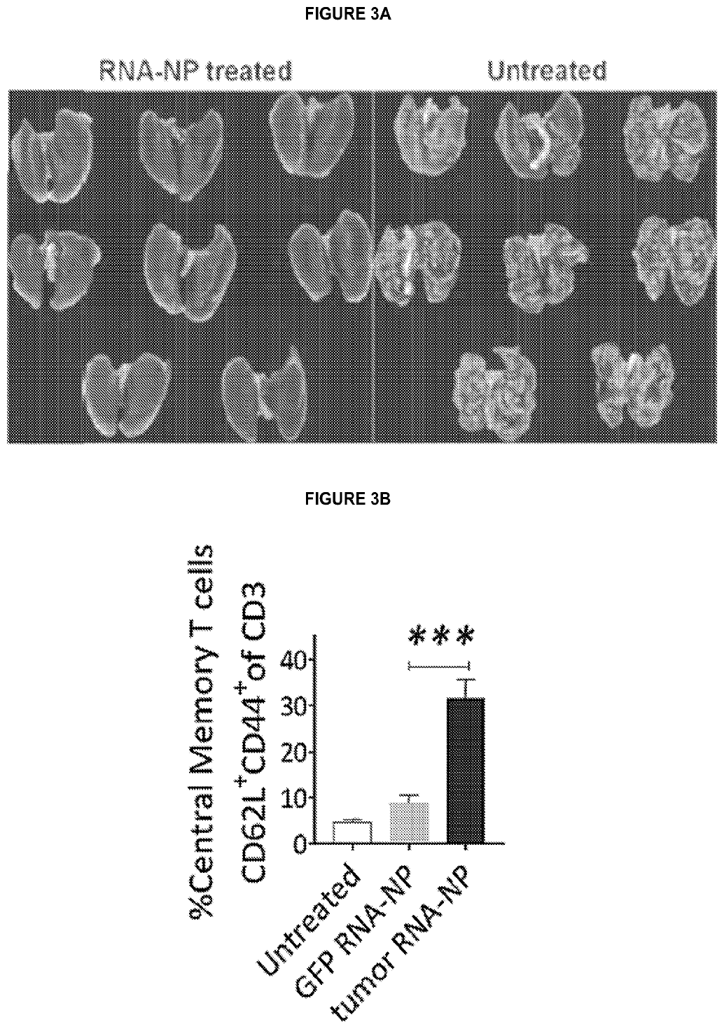

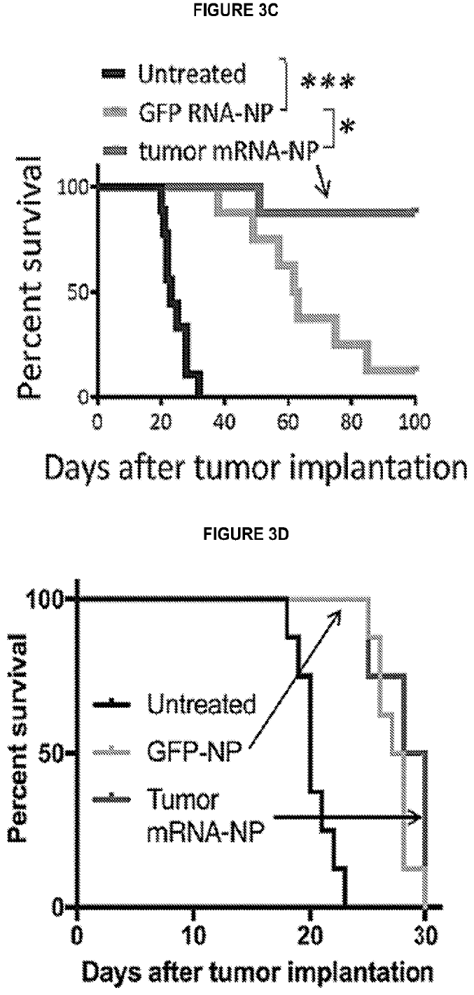

The effect of multilamellar RNA NPs were tested in a second model. Here, BALB/c mice (8 mice per group) inoculated with K7M2 lung tumors were vaccinated thrice-weekly with multilamellar RNA-NPs. A control group of mice was untreated. The lungs were harvested one week after the 3rd vaccine for analysis of intratumoral memory T cells ***p<0.001, Mann Whitney test. FIG. 3A provides a pair of photographs of RNA-NP treated-lungs (left) and of untreated lungs (right). FIG. 3B is a graph of the % central memory T cells (CD62L+CD44+ of CD3+ cells) in the harvested lungs of untreated mice, mice treated multilamellar RNA NPs with GFP RNA, and mice treated multilamellar RNA NPs with tumor-specific RNA.

Also, BALB/c mice or BALB/c SCID (Fox Chase) mice (8 mice per group) were inoculated with K7M2 lung tumors and vaccinated intravenously thrice-weekly with multilamellar RNA-NPs comprising GFP RNA or tumor-specific RNA. A control group of mice was untreated. % survival was plotted on a Kaplan-Meier curve (***p<0.0001, Gehen-Breslow-Wilcox). As shown in FIG. 3C, the percent survival of BALB/c mice treated with multilamellar RNA NPs with tumor-specific RNA was highest among the three groups. Interestingly, the percent survival of BALB/c SCID (Fox Chase) mice treated with multilamellar RNA NPs with GFP RNA was about the same as mice treated with multilamellar RNA NPs with tumor-specific RNA (FIG. 3D).

Taken together, the data of FIGS. 3A-3D demonstrate that monotherapy with RNA-NPs comprising GFP RNA or tumor-specific RNA mediates significant anti-tumor efficacy against metastatic lung tumors in immunocompetent animals and SCID mice. In BALB/c mice bearing metastatic lung tumors (FIGS. 3A-3D), both GFP (control) and tumor specific RNA-NPs mediate innate immunity and anti-tumor activity; however, only tumor specific RNA-NPs mediate increases in intratumoral memory T cells and long-term survivor outcome (FIGS. 3A-3D). Anti-tumor activity of RNA-NPs in mice bearing intracranial malignancies was also demonstrated (data not shown).

These data demonstrate that multilamellar RNA-NPs systemically activate DCs, induce antigen specific immunity and elicit anti-tumor efficacy. FIGS. 3A-3D show that control RNA-NPs elicit innate response with some efficacy that is not as robust as tumor specific RNA-NPs. Compared with untreated mice, no effects of uncomplexed NPs have been observed, but both non-specific (GFP RNA) and tumor-specific RNA when incorporated into multilamellar RNA NPs mediate innate immunity; however only tumor specific RNA-NPs elicit adaptive immunity that results in a long-term survival benefit (FIGS. 3A-3D).

### Example 5

This example demonstrates personalized tumor RNA-NPs are active in a translational canine model.

The safety and activity of multilamellar RNA-NPs was evaluated in canines (pet dogs) diagnosed with malignant gliomas or osteosarcomas. The malignant gliomas or osteosarcomas from dogs were first biopsied for generation of personalized tumor RNA-NP vaccines.

To generate personalized multilamellar RNA NPs, total RNA materials was extracted from each patient's biopsy. A cDNA library was then prepared from the extracted total RNA, and then mRNA was amplified from the cDNA library. mRNA was then complexed with DOTAP lipid NPs, into multilamellar RNA-NPs as essentially described in Example 1. Blood was drawn at baseline, then 2 hours and 6 hours post-vaccination for assessment of, PD-L1, MHCII, CD80, and CD86 on CD11c+ cells. CD11c expression of PD-L1, MHC-II, PDL1/CD80, and PD-L1/CD86 is plotted over time during the canine's initial observation period. CD3+ cells were analyzed over time during the canine's initial observation period for percent CD4 and CD8, and these subsets were assessed for expression of activation markers (e.g., CD44). From these data, it was shown that multilamellar RNA-NPs elicited an increase in 1) CD80 and MHCII on CD11c+ peripheral blood cells demonstrating activation of peripheral DCs and 2) an increase in activated T cells.

Interestingly, within a few hours after administration, tumor specific RNA-NPs elicited margination of peripheral blood mononuclear cells, which increased in the subsequent days and weeks post-treatment; suggesting that RNA-NPs mediate lymphoid honing of immune cell populations before egress.

These data demonstrated that personalized mRNA-NPs are safe and active in translational canine disease models.

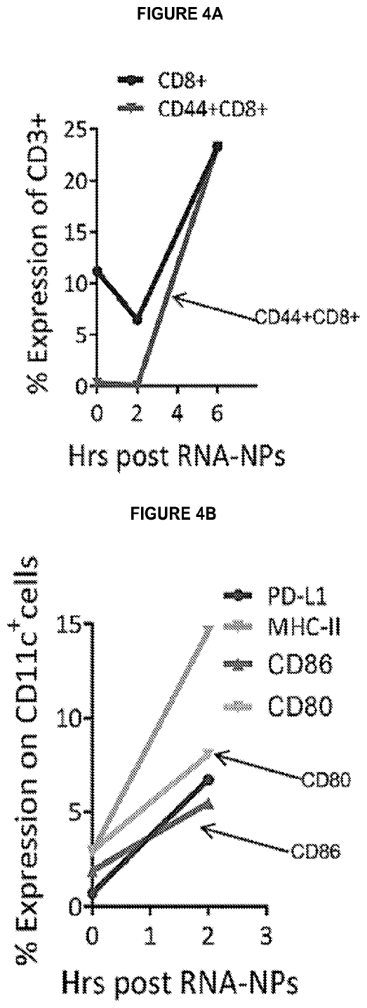

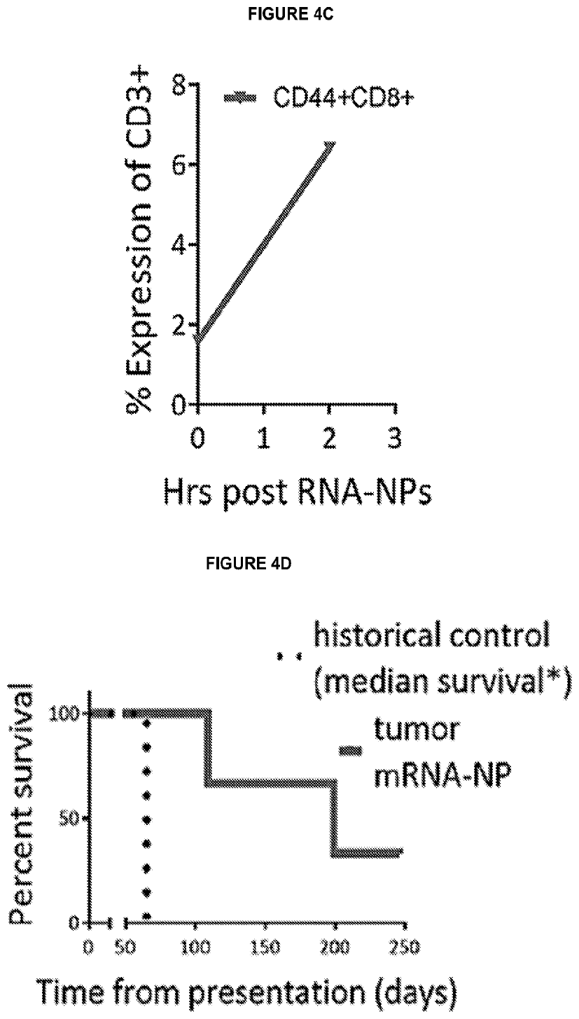

Specific data from canines evaluated in this manner are shown. A 31 kg male Irish Setter was enrolled on study per owner's consent to receive multilamellar RNA-NPs. Tumor mRNA was successfully extracted and amplified after tumor biopsy. Immunologic response was plotted in response to 1^{st} vaccine. The data show increased activation markers over time on CD11c+ cells (DCs) (FIG. 4A). The data show increased CD8+ cells that are activated (CD44+CD8+ cells) within the first few hours post RNA-NP vaccine. These data support that the multilamellar RNA-NPs are immunologically active in a male Irish Setter. A male boxer diagnosed with a malignant glioma was enrolled on study per owner's consent to receive RNA-NPs. Tumor mRNA was successfully extracted and amplified after tumor biopsy. Immunologic response is plotted in response to 1st vaccine (FIG. 4B). The data show increased activation markers over time on CD11c-F cells (DCs). As shown in FIG. 4C, an increase in activated T cells (CD44±CD8±cells) was observed within the first few hours post RNA-NP vaccine. These data support that the multilamellar RNA-NPs are immunologically active in a male canine boxer.

After receiving weekly RNA-NPs (×3), the canines diagnosed with malignant gliomas had a steady course. Post vaccination MRI showed stable tumor burdens, with increased swelling and enhancement (in some cases), which may be more consistent with pseudoprogression from an immunotherapeutic response in otherwise asymptomatic canines. Survival of canines diagnosed with malignant gliomas receiving only supportive care and tumor specific RNA-NPs (following tumor biopsy without resection) is shown in FIG. 4D. In FIG. 4D, the median survival (shown as dotted line) was about 65 days and was reported from a meta-analysis of canine brain tumor patients receiving only symptomatic management. In a previous study, cerebral astrocytomas in canines has been reported to have a median overall survival of 77 days. The personalized, multilamellar RNA NPs allowed for survival past 200 days.

Aside from low-grade fevers that spiked 6 hrs post-vaccination on the initial day, personalized tumor RNA-NPs (1×) were well tolerated with stable blood counts, differentials, renal and liver function tests. It is important to highlight that these canines received no other therapeutic interventions for their malignancies (i.e. surgery, radiation or chemotherapy), and all patients assessed developed immunologic response with pseudoprogression or stable/smaller tumors. The results described above establish the safety and activity of tumor specific RNA-NPs in clinical relevant animal models with malignant brain tumors where the subjects did not receive any other anti-tumor therapeutic interventions.

### Example 6

This example demonstrates toxicology study of murine glioma mRNA and pp65 mRNA encapsulated in DOTAP liposomes after intravenous delivery to C57BL/6 mice.

The objective of this study was to evaluate the safety of pp65 mRNA encapsulated by DOTAP liposomes when delivered intravenously in C57BL/6 mice. Experimental procedures applicable to pathology investigations are summarized in Table 1. All interim phase animals were submitted for necropsy on Day 35±1 day. Tissue samples listed in Table 2 were collected and fixed in 10% neutral buffered formalin (except eye, optic nerve, and testis, which were fixed in modified Davidson's solution); tissues from the early death animal were fixed in 10% neutral buffered formalin.

TABLE 1

| | | Total Dose | Number of Mice | | | | | |
|---|---|---|---|---|---|---|---|---|
| | | (total mRNA + LP; | Day 35 ± 1 day | | Day 56 ± 2 days | | Day 112 ± 3 days | |
| Group | Treatment | (mg/kg) | Males | Females | Males | Females | Males | Females |
| 1 | Vehicle | 0 | 5 | 5 | 5 | 5 | 5 | 5 |
| 2 | LP | 0 + 15.0 | 5 | 5 | 5 | 5 | 5 | 5 |
| 3 | RNA + LP | 0.2 + 3.0 | 5 | 5 | 5 | 5 | 5 | 5 |
| 4 | RNA + LP | 1.0 + 15.0 | 5 | 5 | 5 | 5 | 5 | 5 |

TABLE 2
Tissue Collection and Examination

| | | | Microscopic |
|---|---|---|---|
| Provantis Tissue Term | Protocol Tissue Term | Collect | Evaluation |
| BONE, FEMUR | Femur with bone marrow (R) | X | X |
| BONE MARROW | | X | X |
| BONE, STERNUM | Sternum | X | X |
| BRAIN | Brain stem | X | X |
| | Cerebellum | | |
| | Cerebrum | | |
| EPIDIDYMIS | Epididymis | X | X |
| ESOPHAGUS | Esophagus | X | X |
| EYE | Eye with optic nerve (R) | X | X |
| NERVE, OPTIC | | X | X |
| GLAND, ADRENAL | Adrenal gland (R) | X | X |
| GLAND, PARATHYROID | Thyroid/parathyroid gland | X | X |
| GLAND, THYROID | | X | X |
| GLAND, PITUITARY | Pituitary | X | X |
| GLAND, PROSTATE | Prostate | X | X |
| GLAND, SALIVARY | Salivary gland (R, mandibular) | X | X |
| GLAND, SEMINAL VESICLE | Seminal vesicles | X | X |
| HEART | Heart | X | X |
| KIDNEY | Kidney (R) | X | X |
| LARGE INTESTINE, CECUM | Cecum | X | X |
| LARGE INTESTINE, COLON | Colon | X | X |
| LARGE INTESTINE, RECTUM | Rectum | X | X |
| LIVER | Liver | X | X |
| LUNG | Lungs | X | X |
| LYMPH NODE, MESENTERIC | Lymph node (mesenteric) | X | X |
| MUSCLE, DIAPHRAGM | Diaphragm | X | X |
| MUSCLE, QUADRICEPS | Quadriceps (R) | X | X |
| NERVE, SCIATIC | Sciatic nerve (R) | X | X |
| OVARY | Gonad (Ovary, R) | X | X |
| PANCREAS | Pancreas | X | X |
| SITE, INJECTION | Tail (injection site) | X | X |
| SKIN | Skin | X | X |
| SMALL INTESTINE, DUODENUM | Duodenum | X | X |
| SMALL INTESTINE, ILEUM | Ileum | X | X |
| SMALL INTESTINE, JEJUNUM | Jejunum | X | X |
| SPINAL CORD | Spinal cord, cervical | X | X |
| | Spinal cord, lumbar | | |
| | Spinal cord, thoracic | | |
| SPLEEN | Spleen | X | X |
| STOMACH | Stomach | X | X |
| TESTIS | Gonad (Testis, R) | X | X |
| THYMUS | Thymus | X | X |
| TONGUE | Tongue | X | X |
| URINARY BLADDER | Urinary bladder | X | X |
| UTERUS | Uterus | X | X |
| VAGINA | Vagina | X | X |
| — | Gross lesions | X | X |

Tissues required for microscopic evaluation were trimmed, processed routinely, embedded in paraffin, and stained with hematoxylin and eosin by Charles River Laboratories Inc., Skokie, Ill. Light microscopic evaluation was conducted by the Contributing Scientist, a board-certified veterinary pathologist on all protocol-specified tissues from all animals in Groups 1 and 4, and any early death animals.

Tissues that were supposed to be microscopically evaluated per protocol but were not available on the slide (and therefore not evaluated) are listed in the Individual Animal Data of the pathology report as not present. These missing tissues did not affect the outcome or interpretation of the pathology portion of the study because the number of tissues examined from each treatment group was sufficient for interpretation.

Gross Pathology: No test article-related gross findings were noted. The gross findings observed were considered incidental, of the nature commonly observed in this strain and age of mouse, and/or were of similar incidence in control and treated animals and, therefore, were considered unrelated to administration of a 1:1 ratio of pp65 mRNA and KR158mRNA in DOTAP liposomes.

Histopathology: No test article-related microscopic findings were noted. There were a few animals with inflammatory cell infiltrates at the injection site; this finding is common for injection sites and at this point in the study, was considered equivocal. The microscopic findings observed were considered incidental, of the nature commonly observed in this strain and age of mouse, and/or were of similar incidence and severity in control and treated animals and, therefore, were considered unrelated to administration of a 1:1 ratio of pp65 mRNA and KR158mRNA in DOTAP liposomes.

It was concluded that intravenous injection into the tail vein of mice of 1.0 mg/kg KR158 and pp65 mRNAs+15.0 mg/kg DOTAP liposome on Study Days 0, 14, and 28 resulted in no gross or microscopic test article-related findings on Study Day 35±1 day. There were small amounts of inflammatory cell infiltrates at the injection site, which is a common finding for injection sites. This finding was equivocal.

### Example 7

This example demonstrates non-antigen specific multilamellar (ML) RNA NPs mediate antigen-specific immunity long enough to confer memory and fend off re-challenge of tumor.

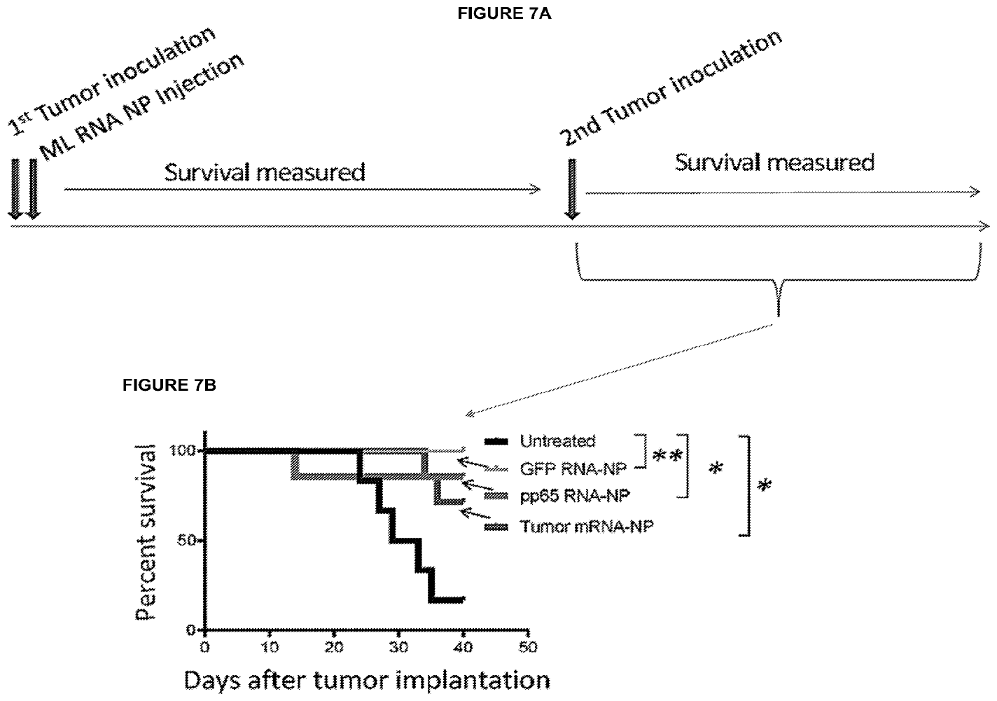

An experiment was carried out with long-term surviving mice (e.g., mice that survived for ˜100 days) that were challenged a total of two times via tumor inoculation, but treated only once weekly (x3) with ML RNA NPs comprising GFP RNA or pp65 RNA (each of which were non-specific to the tumor) or with ML RNA NPs comprising tumor-specific RNA. The treatment occurred just after the first tumor inoculation and about 100 days before the second tumor inoculation. Because none of the control mice (untreated mice) survived to 100 days, a new control group of mice were created by inoculating the same type of mice with K7M2 tumors. The new control group like the original control mice did not receive any treatment. The long-time survivors also did not receive any treatment after the second time of tumor inoculation. A timeline of the events of this experiment are depicted in FIG. 7A.

Remarkably, mice in all 3 groups contained long-time survivors that survived the second tumor challenge. As shown in FIG. 7B (which shows only the time period following the 2^{nd} inoculation), mice in all 3 groups contained long-time survivors with survival to 40 days post tumor implantation (second instance of tumor inoculation). Interestingly, the percentage of long-time survivor mice that were previously treated with ML RNA NPs comprising non-specific RNA (GFP RNA or pp65 RNA) survived to 40 days post second tumor inoculation, comparable to the group treated with ML RNA NPs comprising tumor specific RNA (treated before second tumor challenge).

These data support that ML RNA NPs comprising RNA non-specific to a tumor in a subject provides therapeutic treatment for the tumor comparable to that provided by ML RNA NPs comprising RNA specific to the tumor, leading to increased percentage in animal survival.

### Example 8

This example describes studies showing that infiltrative and chemotherapy-resistant slow-cycling cells contribute to metabolic heterogeneity in glioblastoma.

Metabolic reprogramming, known as the Warburg effect, has been described in rapidly growing tumors, which have been thought to mostly contain fast-cycling cells (FCCs) with impaired mitochondrial function and relying on aerobic glycolysis. GBM tumors are metabolically heterogeneous; FCCs harness aerobic glycolysis and slow-cycling cells (SCCs) utilize mitochondrial oxidative phosphorylation for cell proliferation and survival. Hoang-Minh et al., EMBO J 37(23):e98772 (doi: 10.15252/embj.201798772). SCCs display enhanced invasion and chemoresistance, suggesting their significant role in tumor recurrence. SCCs also demonstrate increased lipid content that is specifically metabolized under glucose-deprived conditions. Those lipid stores are surrounded by a rich network of autophagosomes/lysosomes, which are involved in catabolic pathways providing energy to cells in response to decreased nutrient availabilities. Furthermore, SCCs show increased fatty acid transport that is prevented by fatty acid binding protein pharmacological inhibition or gene knockout, both of which sensitize those cells to metabolic stress. GBM cell subpopulations with distinct metabolic requirements are targetable candidates for the inhibition of highly infiltrative and treatment-resistant SCCs.

Our studies revealed the following: SCCs display migration, invasion, and chemoresistance characteristics that promote GBM recurrence; treatment-resistant/recurrent tumors share metabolic gene signatures with SCCs; SCCs display increased mitochondrial activity and OxPhos; a metabolic dichotomy exists between GBM SCCs and FCCs; lipid metabolite levels are increased in SCCs; lipid droplets constitute a form of energy storage in SCCs; fatty acid transport is facilitated in SCCs; and SCCs' resistance to metabolic stress is driven by FABP7-dependent exogenous fatty acid uptake. Hoang-Minh et al., 2018, supra. Preventing lipid metabolism through fatty acid transport inhibition blocks the ability of SCCs to survive metabolic stresses such as glucose restriction and could be effective at least in some GBM tumors, if not all, due to the tumors' heterogeneity.

The Warburg effect is observed in GBM and other tumor types. GBM is metabolically heterogeneous, with FCCs relying on aerobic glycolysis and SCCs depending on mitochondrial OxPhos for their survival and proliferation. It was found that, compared to FCCs, SCCs contain increased levels of lipid metabolites and components that are involved in lipid metabolism, storage, and transport. These properties provide SCCs with a survival advantage when these cells are exposed to metabolic stresses such as glucose deprivation. Indeed, the resistance of SCCs to glucose deprivation can be prevented by blocking the uptake of fatty acids through the inhibition of fatty acid transporter FABP7. Interestingly, the specific metabolic characteristics of SCCs are accompanied by increased chemotherapy resistance and migration/invasion when compared to the rest of the tumor cell population.

Our data showed that SCCs possess greater migration and invasion capabilities as well as higher resistance to TMZ than FCCs (Deleyrolle L P, et al. (2011) Brain 134:1331-43). Previous studies reported that an EMT-like process positively correlates tumor cell invasion and chemoresistance (Siebzehnrubl F A, et al. (2013) EMBO Mol Med:1196-1212) (Qi et al., 2012, PLoS One 7:e38842) (Depner et al., 2016, Nat Comm 7:12329) and that quiescent GBM cells are more chemoresistant (Chen et al., 2012, Nature 488:522-526) (Campos et al., 2014, J Pathol 234:23-33). Interestingly, our studies revealed that the knockdown or overexpression of the EMT transcription factor, ZEB1, affects GBM cell proliferation, with greater ZEB1 levels reducing proliferation and enhancing cellular invasion. Our results show higher expression of ZEB1 and greater resistance to chemotherapy in GBM SCCs, suggesting a positive correlation between tumor cells' ZEB1 expression, invasion, chemoresistance, and slow proliferation.

Our characterization of the nature of GBM's metabolic heterogeneity provides an actionable target for immunotherapy.

### Example 9

This example describes materials and methods for exploring the heterogeneity of GMB, as described in Hoang-Minh et al., 2018, supra.

Cell culture: The primary cell lines used in this study, Line 0 (L0), Line 1 (L1) and Line 2 (L2) (Deleyrolle L P, et al. (2011) Brain 134:1331-43; Siebzehnrubl F A, et al. (2013) EMBO Mol Med:1196-1212), were isolated from human GBM tumors and cultured as previously described (Deleyrolle L P, et al. (2011) Brain 134:1331-43; Hoang-Minh L B, et al. (2016) Oncotarget 7:7029-43, Sarkisian M R, et al. (2014) J Neurooncol. 117:15-24; Siebzehnrubl F A, et al. (2013) EMBO Mol Med:1196-1212; Siebzehnrubl F A, et al. (2011) Methods Mol Biol 750:61-77). The lines were authenticated using STR analysis (University of Arizona Genetics Core). Cells were grown as floating spheres and maintained in Neurocult NS-A medium (StemCell Technologies) in the presence of 20 ng/mL human EGF. When the spheres reached approximately 150 μm in diameter, they were enzymatically dissociated by digestion with Accumax (Innovative Cell Technologies, Inc.) for 10 min at 37° C. Cells were then washed, counted using Trypan blue to exclude dead cells, and re-plated in fresh complete medium. To generate TMZ-resistant cells, cells were initially treated with 500 μM TMZ for one passage and then continuously exposed to 20 μM TMZ.

Isolation of fast- and slow-cycling cells: Populations of slow-cycling cells (SCCs) and fast-cycling cells (FCCs) were identified and isolated primary human glioblastoma cell lines based on their capacity to retain CellTrace dyes (Carboxyfluorescein succinimidyl ester-CFSE or Cell Trace Violet-CTV, Invitrogen), as described previously (Deleyrolle L P, et al. (2011) Brain 134:1331-43), and grouped as CFSE/Violet^{high}—top 10% and CFSE/Violet^{low}—bottom 10%. FCCs were isolated as CFSE^{low}—bottom 85% (Deleyrolle L P, et al. (2011) Brain 134:1331-43). The gating strategy allows for the isolation of functional and phenotypic extremes with similar size population, homogenizing for sorting time and hence overcoming the issue of fluorescence-activated cell sorting (FACS)-related metabolic stress. Both SCC and FCC populations are able to expand in vitro and in vivo, demonstrating their viability and expansion capacities (Deleyrolle L P, et al. (2011) Brain 134:1331-43). Although this strategy does not capture the full spectrum of cellular population contained in GBM, it provides a relevant paradigm to compare defined cellular components or states with distinct proliferation properties. Proliferation was assessed based on CellTrace fluorescence intensity decay rate over time measured by flow cytometry and identified 6-8 days post labeling. This process enabled the separation of rapidly proliferating (FCCs) and slowly proliferating (SCCs) cell fractions (top and bottom 10%) based on CellTrace fluorescence intensity, which is proportional to dye dilution. These most extreme fractions of the proliferation spectrum were used in order to ensure clear and distinct separation of FCCs and SCCs based on cell cycle kinetics. All experiments were performed immediately after FACS of those SCC and FCC populations.

Scratch assay: Sorted SCCs and FCCs were plated as described previously (Siebzehnrubl F A, et al. (2013) EMBO Mol Med:1196-1212) at 2 million cells per well of a six-well plate pre-coated with poly-D-lysine and laminin, in medium containing 1% fetal bovine serum. Twenty-four hours after plating, a scratch was made with a 200-μL pipette tip. Cells were imaged at the time of lesion, as well as 24 hours later, and the distance traveled by the most migratory cells was recorded.

Migration Assay: For quantification of cell migration, tumor spheres were plated onto a laminin/poly-D-lysine coated surface at low density and in the presence of growth factors, FABP7 inhibitor (SB-FI-26), or DMSO as a solvent control. Images were taken from the same spheres 2 hours and 24 hours after plating with a Leica DM IL microscope equipped with a DFC3000G camera and Leica application suite X software. The greatest distance of outgrowing cells was measured using ImageJ, and migration distance was calculated as the difference between the two time points. Only spheres with a diameter greater than 50 μm, 2 hours after plating, were used to measure migration distance.

In vivo invasion quantification: To quantify the effects of ZEB1 on the invasion of SCC, GBM cells were stably transfected with shZEB1 or shControl constructs as described (Siebzehnrubl F A, et al. (2013) EMBO Mol Med:1196-1212). After selection, transfected cells were loaded with CFSE and separated into SCC and FCC fractions, and each intracranially injected into 5 SCID mice as described (Siebzehnrubl F A, et al. (2013) EMBO Mol Med:1196-1212). Mice were transcardially perfused 12 weeks after implantation, their brains harvested and post-fixed in 4% formalin overnight. Brains were sectioned and stained and analyzed using the Invasion Index as described (Siebzehnrubl et al., 2013).

To assess the intrinsic invasion capacity of SCC and FCC populations, sorted SCC/FCC populations were allowed to recover for 24 hours in culture and then transduced with lentiviral vectors encoding for humanized eGFP (SCCs) or humanized RFP (FCCs) (both kind gift of Dr Lung-Ji Chang, University of Florida). In both cases, the transduction efficiency was greater than or equal to 95%. Cells were allowed to expand and then dissociated into single cells and mixed at a ratio of 1:1. 10⁵ cells of this mixture were intracranially implanted into SCID mice. Mice were transcardially perfused 6 weeks after implantation and their brains harvested and post-fixed in 4% formalin overnight. Brains were sucrose-protected, frozen, and sectioned. Sections were counterstained with Hoechst 33342, mounted onto slides, and imaged for GFP and RFP using an Olympus BX-81 DSU spinning-disk confocal microscope and SlideBook software.

Cell viability/proliferation assays: The methyltetrazolium bromide (MTT) assay was used as an indicator of cell viability and performed as described (Siebzehnrubl F A, et al. (2013) EMBO Mol Med:1196-1212). Briefly, 2000-5000 cells were plated per well in 96-well plates, in medium containing 1% fetal bovine serum. The cell populations were treated with TMZ one day after plating and analyzed with MTT assay 96 hours later. Bar graphs were derived from individual concentration measurements, which were compared to the appropriate controls.

Propidium iodide incorporation and expression of cleaved caspase 3 were used to compare the effects of glucose restriction and/or mitochondrial function inhibition (by rotenone or metformin treatment). Briefly, cells were labeled with CellTrace dye and grown in complete medium for 5-7 days before being placed in high glucose (HG; >500 mg/dL) or physiological glucose conditions (PG; 90-110 mg/dL) and/or treated with rotenone (0.5-1 μM) or metformin (10-20 mM) for 24 hours. Media glucose concentrations were monitored daily and maintained constant throughout the experiments by adding glucose to the cell cultures as needed, which prevented the glucose supply exhaustion that might have occurred due to FCCs' higher division rate.

Propidium iodide and cleaved caspase 3 staining were quantified using flow cytometry. The effects of restricting glucose along with mitochondrial targeting using rotenone or metformin were investigated using the CyQUANT™ assay. Cells were plated at 60,000 cells per well in 96-well plates and exposed to the treatments alone or in combination (physiological glucose, 0.5 μM rotenone, and 10 mM metformin). CyQUANT™ binding dye was added to each well and incubated for 30 min at 37° C. before being quantified using Biotek™ Cytation™ 3 Cell Imaging Multi-Mode Reader.

Assessment of mitochondrial function: Cells were seeded at a density of 30,000 cells in 80 μL medium per well in XF96-well microplates (Seahorse Bioscience) (n=10) pre-coated with 22.4 μg/mL Cell-Tak Adhesive (Corning). SCCs and FCCs were incubated for 24 hours in standard growth medium in a humidified incubator at 37° C. with 5% CO₂. After 24 hours, the standard medium was exchanged for XF Base Medium pH 7.4 (Seahorse Bioscience) supplemented with 25 mM glucose, 2 mM L-glutamine, and 1 mM sodium pyruvate. The cells were then incubated for 1 hour at 37° C. without CO₂. OCRs were measured using the XF Cell Mito Stress Assay (Seahorse Bioscience) and prior to and following additions of the following: 1) ATP synthase inhibitor (1 μM oligomycin), 2) uncoupler (1 μM carbonyl cyanide 4-(trifluoromethoxy)phenylhydrazone (FCCP)), and 3) Complex I/II inhibitors (0.5 μM Rotenone/Antimycin A). Data were analyzed using Wave Desktop Software (Seahorse Bioscience), following the manufacturer's instructions, and normalized to protein levels.

ATP level measurement: ATP levels were measured using the luciferase-based ATP-lite assay (Perkin Elmer) as per the manufacturer's instructions. Briefly, 10,000 SCCs or FCCs were seeded per well (n=10) of a black-walled 96-well tissue culture plate. Luminescence (indicative of intracellular ATP levels) was measured using a Spectra Max i3x microplate reader (Molecular Devices) and normalized to protein levels for each well.

Electron Microscopy: SCCs and FCCs were separated by FACS before being fixed with 2.5% glutaraldehyde in 0.1 M cacodylate buffer (pH 7.4) overnight and washed with 0.1M cacodylate buffer again. Cells were then postfixed in 1% osmium tetroxide for 1 hour before additional buffer washes. Cells were dehydrated through an ethanol series followed by 3 additional 100% ethanol. Subsequently, cells were infiltrated with a mixture of 100% ethanol and Eponate 12 resin (Ted Pella Inc., Redding, Calif.) and then pure Eponate 12 resin overnight. Cells were embedded in Eppendorf tubes and then placed in a 60° C. oven for polymerization. Ultrathin 70-80 nm-thick sections were cut on a Leica UltraCut microtome. Sections were then stained with 5% uranyl acetate for 15 minutes followed by 2% lead citrate for 15 minutes. Mitochondria were imaged with a JEOL JEM-1400 transmission electron microscope (Tokyo, Japan) equipped with a Gatan US1000 CCD camera (Pleasanton, Calif.). The data described here were gathered on the JEOL JEM-1400 120 kV TEM supported by a National Institutes of Health Grant S10 RR025679.

Generation and maintenance of FABP7-depleted cell lines: To generate FABP7-depleted patient-derived GBM cell lines, we screened and identified CRISPR/Cas9-encoding plasmids containing a GFP reporter gene that could target human FABP7 [Sigma-Aldrich; CRISPR/Cas-GFP vector (pU6-gRNA-CMV-Cas9:2a:GFP); primer pair ID: HS0000240647; FABP7 gRNA target sequence: CTTGACTGATAATTACCGT (SEQ ID NO: 1]. For transfection experiments, GBM cells were grown on 10-cm2 plates and transfected (Lipofectamine 2000; Life Technologies) at 60% to 70% confluence with 0.5 μg/ml of the CRISPR/Cas9-encoding plasmid DNA. Twenty-four to 48 hours after transfection, GFP-positive cells were sorted as individual clones into 96-well plates containing 250 μl of complete medium supplemented with hEGF using a BD FACS Aria II Cell Sorter (BD Biosciences, San Jose, Calif.), excluding cell debris and dead cells from the analysis by forward- and side-scatter gating and PI exclusion. Stable cell lines from each GFP-positive clone were then expanded and screened for the presence of FABP7 by immunofluorescence microscopy analysis as well as flow cytometry. GFP-positive clones with undetectable FABP7 levels were designated CRISPR FABP7 (crFABP7, L1 clone H7). For immunostaining, once cells formed spheres greater than 100 μm in diameter in each well, the spheres were mechanically dissociated, replated, and expanded into Labtek chambered slides in 5% FBS-supplemented complete medium. After 2 to 3 days, cells were fixed with 4% paraformaldehyde in 0.1 M phosphate buffer (4% PFA) for immunohistochemical analysis as described below.

Animal experiments: Adult male NOD-SCID mice (7-15 weeks old) were used for in vivo tumor implants following NIH and institutional (IACUC) guidelines and regulations for animal care and handling. The mice colonies were maintained at the University of Florida's animal facility. Animals were randomized to cages following implantation. For in vivo tumor invasion assay, FACS cells were intracranially implanted as previously described (Deleyrolle L P, et al. (2011) Brain 134:1331-43; Siebzehnrubl F A, et al. (2013) Hoang-Minh et al., EMBO Mol Med:1196-1212) and invasion assay was performed 10 weeks post implant. For in vivo TMZ treatment, animals were implanted with 100,000 cells immediately after cell sorting. Tumor-bearing animals were intraperitoneally treated with 5 injections of 20 mg/kg TMZ over 5 days at 3 (hGBM L0) or 4 (hGBM L2) weeks post implantation. For in vivo restricted glucose and mitochondria targeting experiments, animals were xenografted with SCCs or FCCs and subjected to either a high carbohydrate control diet or a custom supplemented high fat/low carbohydrate dietary regimen (sHFLC) (Martuscello R T, et al. (2016) Clin Cancer Res. 22:2482-95). Each group received vehicle or rotenone treatment (0.5 mg/kg i.p, once a week for 6 weeks).

Mass spectrometry-based metabolite screening: CellTrace-labeled cells were cultured in gliomasphere growth conditions for 5-7 days before being separated into SCCs and FCCs using FACS. Upon isolation, cells were placed into 10 mM ammonium acetate for metabolic fingerprinting using UHPLC/HRQMS. Detected metabolites were identified based on both retention time and mass accuracy using major metabolite databases, including the Human Metabolome DataBase (HMDB), Madison Metabolomics Consortium Database (MMCD), Metlin, LIPID MAPS, and our 700 compound internal library (from the Southeast Center for Integrated Metabolomics). For final identification, tandem MS was performed to confirm assignment. Statistical analyses were performed using JMP 11 and Metaboanalyst, (www.metaboanalyst.ca), a free R-based metabolomic statistical analysis package. In addition, multivariate statistics including principal components analysis (PCA) and partial least squares-discriminant analysis (PLS-DA) were used to identify metabolites that might differentiate the cell lines or cellular subtypes.

Lipid uptake: Each cell line was labeled with CellTrace dye and grown for 5-7 days. Cells were then dissociated and treated with different BODIPY®FLC16 (Molecular probes) concentrations and incubation times described below. Fatty acids conjugated to C16-BODIPY fluorophore undergo native-like metabolism and transport. Dose response was performed with 0, 0.5, 2.5, 5, 10, 25, and 50 nM BODIPY, and the time course was done at a concentration of 5 nM for 1, 4, 5, 10, and 15 min. The cells were then washed and fixed with 4% paraformaldehyde. To determine the amount of fatty acid uptake, cells were analyzed by flow cytometry on a BD LSRII Flow Cytometer. Fatty acid transporter inhibitors SB-FI-26 (Cayman Chemicals, #14191) and BMS309403 (Millipore, #34310) were used to inhibit FABP7 (Kaczocha et al., 2014) and FABP3 (Furuhashi et al., 2007), respectively.

Dyes and antibodies: Dyes and primary antibodies used for flow cytometry or immunocytochemistry included CellTrace™ Violet and CFSE Cell Proliferation Kit (Molecular Probes), DAPI (Molecular Probes), Hoechst (Thermo Scientific), DRAQ5 (Thermo Scientific), propidium iodide (Molecular Probes), LipidTox (ThermoFisher Scientific), MitoTracker Green and Orange (Molecular Probes), MitoProbe DilC1(5) (Molecular Probes), FABP7 (Santa Cruz Biotechnology, #sc-30088), FABP7 (R&D Systems, #AF3166), human Nestin (Millipore, #MAB5326), N-cadherin (Millipore, #04-1126), beta-catenin (Sigma, #C2206), ZEB1 (Sigma, #HPA027524), VDAC1 (Abcam, #ab15895), cleaved caspase-3 (Cell Signaling Technology, #9661S), NDUFA4 (Abcam, # ab129752), ATP synthase (BD Biosciences #612518), LC3 (Cell Signaling Technology, #3868S), and LAMP2 (Abcam, #25631) antibodies.

Flow cytometry: Six to 8 days post-CellTrace load, labeling was performed using the antibodies and dyes that are listed above and according to the manufacturer's protocol. Staining was quantified by flow cytometry (BD LSRII) and percent of immunoreactive cells or mean fluorescence intensity (MFI) were reported.

Image acquisition and invasion measurement: Tumor invasion was measured using human-specific nestin labeling (Millipore, MAB5326). Full images of brain sections were obtained by multiple gray scale imaging acquired using Spot Advanced software (Spot Imaging Solutions), merged into full images, and inverted into black-and-white images using Photoshop CS6 (Adobe Systems). Staining threshold levels were adjusted in Image J software to distinguish tumor from background, as previously described (Siebzehnrubl F A, et al. (2013) EMBO Mol Med:1196-1212). Invasion index was obtained by calculating the ratio of the squared-perimeter distance over the area (P2/A). Dissociated tumors are associated with higher invasion indices compared to more spherical tumors characterized by lower invasion indices. High-power images of stained tissues were taken using an IX81-DSU spinning disk confocal microscope (Olympus) fitted with a 60× water immersion objective, and all images were captured as z-stacks (0.5 μm steps). For 3D imaging, pictures were acquired using a UPLSAPO 60× water objective and Hamamatsu ORCA-AG Camera. Images were captured as z-stacks (0.5 μm steps). All image analyses and 3D surface reconstructions utilized the 3i SlideBook v4.2 Software (with Deconvolution Module). Image capture settings were standardized across samples. 3D surface reconstruction rendering cut-off values were also standardized in the 3i SlideBook software.

RNA sequencing and GSEA: The autophagosome-lysosome gene set was compiled by combining the list of genes from The Human Lysosome Gene Database (http://lysosome.unipg.it/index.php) and the GO Autophagosome gene set (software.broadinstitute.org/gsealmsigdb/cards/GO_AUTOPHAGOSOME.html). Enrichment of the gene signature was assessed using GSEA (www.broadinstitute.org/gsea/index.jsp), and p values were obtained by permuting the phenotypes (1000 permutations). To broaden the validity of the gene signature enrichment, additional gene sets were used based on previously published autophagosome-lysosome gene signatures (Perera R M, et al. (2015) Nature 524:361-5; Jegga A G, et al. (2011) Autophagy 7:477-89). The stem cell signature was derived from (Wong et al., 2008). GBM single-cell RNA sequencing data were generated from (Venteicher A S, et al. (2017) Science 355). Differentially expressed genes were extracted from groups by nonparametric t-test (p<0.05). Gene set enrichment analysis was performed using GenePattern ssGSEA.

Bioinformatics analysis: Using information from the TCGA dataset (Cancer Genome Atlas Research Network, 2008), we analyzed the RNA expression of 155 primary (de novo) and 14 recurrent patients' GBM tumors. All 20,530 identified genes were normalized by log 2 transformation and centered by mean value. Results were presented using a volcano plot comparing recurrent and primary GBM gene expression using a threshold of a 2-fold change with p<0.05 (Mann-Whitney U-test, Subio platform) as significant difference. For pathway analyses, we used the Search Tool for the Retrieval of Interacting Genes/Proteins (STRING) database platform (Szklarczyk D, et al. (2015) Nucleic Acids Res. 43:D447-52) to identify functional networks differentially activated between primary and recurrent tumors. The datasets that were utilized are indicated in the text, Figure legends, and on the Figures of Hoang-Minh et al., 2018, supra. TOGA data were accessed.

Quantitative RT-PCR: SCCs and FCCs were isolated from cell lines L0, L1, and L2 as described above. RNA was extracted using Trizol. After treatment with RNase-free DNase I, cells from each group were purified using the RNeasy Mini Kit (Qiagen). RNA quantity and purity were determined using a NanoDrop ND-1000, and RNA integrity was assessed by determining the RNA integrity number and 28S/18S ratio using a Bioanalyzer 2100 (Agilent Technologies). A quantity of 500 ng of high-quality RNA (260/280 ratios slightly higher than 2.0 and 260/230 ratios higher than 1.7) for each group was converted into cDNA using the RT2 First Strand cDNA Kit (SABiosciences). All qPCR reactions used the RT2 SYBR Green qPCR Master Mix (SABiosciences). Fatty acid metabolism gene expression was determined using the Fatty Acid Metabolism PCR Array (PAHS-007Z, SABiosciences), and the C100 Touch Thermal Cycler CFX96 Real-Time System (Bio-Rad) according to the manufacturer's protocol. FABP7 expression levels were assessed. For additional glucose metabolism gene analyses, LDH-A, B, and C expression levels were detected using the C100 Touch Thermal Cycler CFX96 Real-Time System (Bio-Rad) with Actb as control. Primers were purchased from ThermoFisher Scientific.

Statistics: Values reported in the results are mean values+/− SEM, and statistical analyses were performed using GraphPad Prism 6.0 (GraphPad Software). Statistical tests are indicated in the text. Comparisons between groups were performed appropriately using either a one-way ANOVA or Student's t-test (95% confidence intervals). Groups that showed significant differences with ANOVA were further subjected to Tukey's post-hoc analysis. In vivo survival analyses were calculated using log-rank analyses. Flow cytometry analysis was performed using FlowJo software. Generalized linear models (GLM) were used with log-normal errors (McCullagh & Nelder, 1989) to analyze the effect of experimental factors on mean responses. All experiments analyzed in this way involved at least two experimental factors. Corresponding models included design variables representing the main effects and interactions among factors. Survival time responses were converted to “pseudo-observations” that more accurately represented the contributions of observed and right-censored survival times to unbiased survival time mean estimates (Klein et al, 2008). GLM models incorporating a robust “sandwich” estimator for the covariance matrix (equivalent to generalized estimating equation models) were fitted to pseudo-observation survival times. Residuals from model fits were evaluated graphically to assess model fit assumptions. F tests were used to test the significance of interactions and main effects. Means and 95% confidence intervals (CI) were estimated for various experimental conditions. Percent differences between means, and percent differences between the effects of experimental factors represented by interactions, were estimated and tested for significant difference from zero using t statistic contrasts within the framework of fitted models. Via repeated simulation of responses within our various experimental designs, we determined that we had 80% power to detect 49-55% percent differences between effects within interactions at a 2-sided significance level of 0.05. Model-fitting and estimation was carried out using SAS Version 9.4 (SAS Institute, Cary, N.C., USA). Data simulation and retrospective power calculations were carried out using R Version 3.5.0 (R Foundation for Statistical Computing, Vienna, Austria).

Supplementary Tables 1A and 2-11 was published on Oct. 15, 2018, in Hoang-Minh et al., “Infiltrative and drug-resistant slow-cycling cells, support metabolic heterogeneity in glioblastoma.” EMBO J (2018) e98772, hereby incorporated by reference.

### Example 10

This example demonstrates personalized slow-cycling tumor RNA based nanoparticle vaccine to treat cancer.

Conventional therapies most effectively eliminate rapidly dividing cells but spare slowly dividing populations. The existence of slow-cycling cells that exhibit enhanced tumorigenicity and resistance to therapy in high-grade glioma has been demonstrated (Deleyrolle et. al., 2011). Clinical strategies able to target this specific phenotype hold great promises in improving prognosis. Due to their intrinsic resistance to conventional treatments, their infiltrative propensity, and their ability to initiate recurrent disease, glioblastoma slow-cycling tumor-initiating stem cells represent an ideal target for directed therapeutics.

The use of a nanoparticle (NP) vaccine engineered with RNA derived from a specific subpopulation of slow-cycling tumor cells is contemplated. In brief, slow-cycling tumor-initiating stem cells are identified via their ability to retain a specific label obtained by treating tumor cells with CellTrace dye in specific culture conditions. Subsequently, the cells are FACS-sorted before extracting their total or messenger RNA, which is then complexed at definite ratios with nanoparticles (DOTAP) via specific sonication protocol forming unique cationic lipoplexes. Once created, the nanoparticle vaccine is injected i.v. at given doses and frequency in subjects bearing tumors. This therapeutic platform (slow-cycling cells-based RNA-NPs) is able to activate T cell recognition against this clinically relevant target (slow-cycling tumor-initiating stem cells) mediating sustained anti-tumor activity.

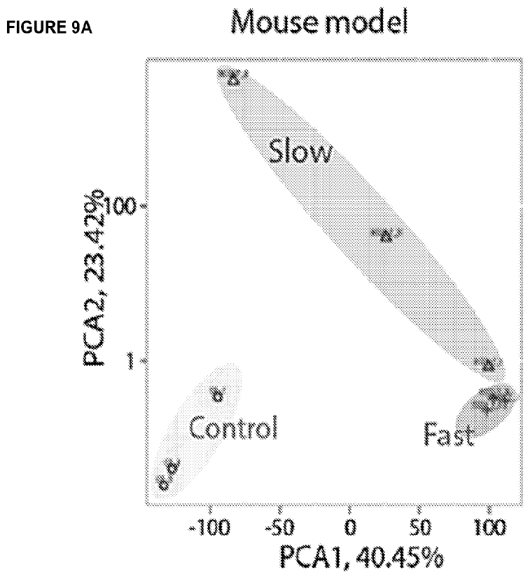

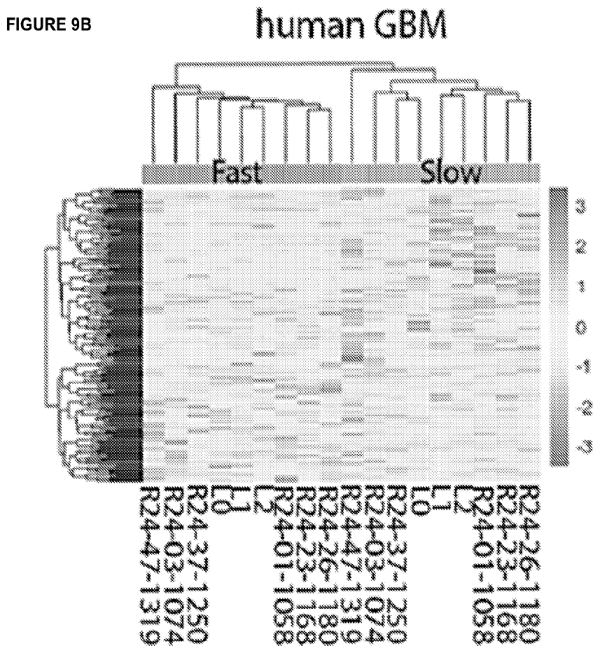

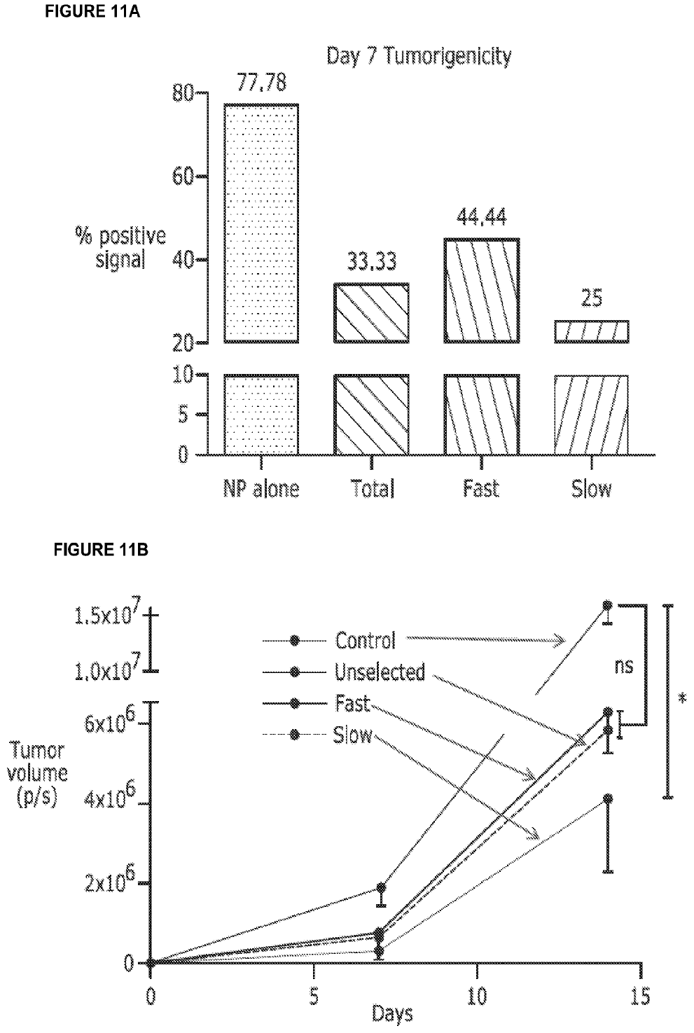

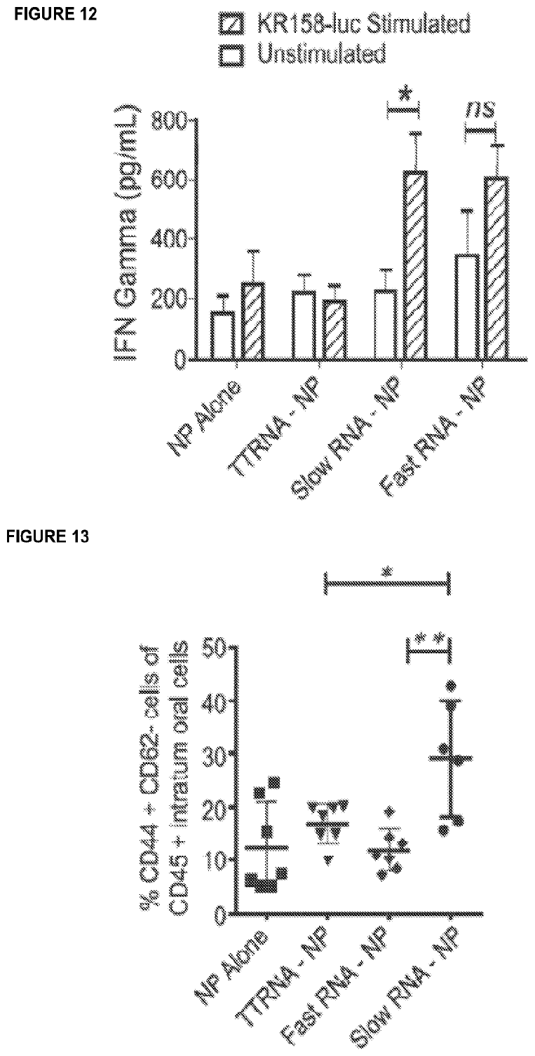

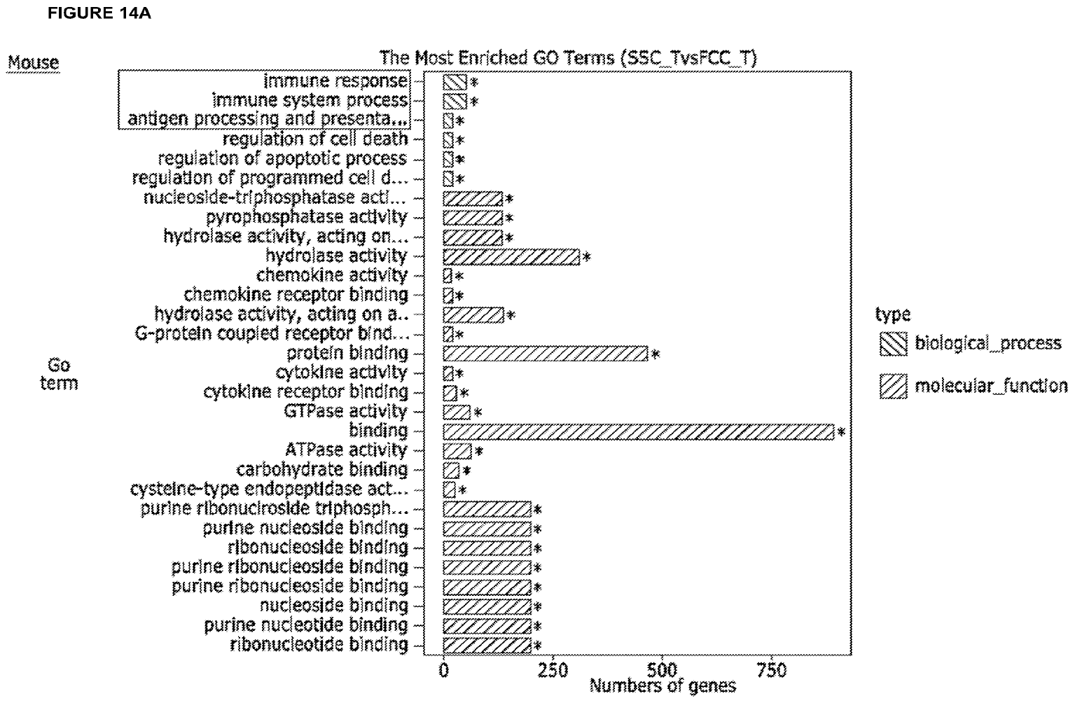

Transcriptome analysis using RNA sequencing was performed to compare gene expression between slow and fast-cycling cells derived from a mouse model of glioma (KR158). FIG. 9A reveals differential RNA expression between slow and fast-cycling cells from a mouse model of glioma. PCA analysis of RNA sequencing data shows differential expression between slow vs fast-cycling cells in a mouse model of glioma. Control represents adult mouse normal astrocytes. FIG. 9B shows gene expression between slow and fast-cycling cells in human GBM patients. These results indicate different specific RNA antigens between slow vs fast-cycling cells. To test the hypothesis that mouse glioma slow-cycling cell-derived RNA vaccine provides preferential anti-tumor advantages, a treatment platform based on the use of nanoliposomes (NP) to deliver tumor RNA to dendritic cells was used. Results demonstrated increased tumor cell targeting from splenic white blood cells of non-tumor bearing animals vaccinated with slow-cycling cells RNA-NPs compared to fast cycling RNA-NPs and TTRNA-NPs (FIG. 10). Superiority of this vaccine strategy was confirmed in vivo as seen by decreased tumorigenicity (FIG. 11A) and tumor growth overtime (FIG. 11B) in animals vaccinated with slow-cycling cells RNA-NPs. Only slow cycling RNA-NPs mediated antigen specific T cell responses (FIG. 12) with increased intratumoral effector/memory tumor infiltrating lymphocytes (TILs) (FIG. 13).

To demonstrate the use of the presently disclosed nanoparticles to deliver tumor RNA to dendritic cells, the above-described mouse study is repeated with RNA NPs made as essentially described in Example 11 or Example 12. Similar if not better results are expected with the nanoparticles of the present disclosure.

### Example 11

This example demonstrates a method of making a personalized RNA-NP using RNA from SSCs isolated from a mixed population of tumor cells and a method of administering the same to the patient.

RNA Preparation: Prior to incorporation into nanoparticles, RNA is prepared from a mixed population of tumor cells as follows:

A sample of a tumor is obtained from a human subject diagnosed with glioblastoma via biopsy and processed as essentially described in Deleyrolle et al. (2011), supra, to obtain a mixed tumor cell population. Briefly, after surgical removal, the biopsied tissue is washed and mechanically dissociated before being placed in an enzymatic cocktail containing trypsin/ethylenediaminetetraacetic acid (0.05%) for 10 min at 37° C., followed by filtration through a 40-μm filter. Dead cells are quantified using trypan blue labelling and the cells are then transferred (at a density of 50 000 viable cells per ml) into neurosphere assay growth conditions. Under these culture conditions, the tumor cells generate gliomaspheres that can be serially passaged. When the gliomaspheres have reached an adequate size (˜150 μm diameter), they are dissociated using enzymatic digestion with a solution containing trypsin/ethylenediaminetetraacetic acid (0.05%) for 3-5 min. Cells are washed, counted using trypan blue to exclude dead cells and replated in fresh media supplemented with epidermal growth factor and basic fibroblast growth factor.

SSCs are isolated from the mixed population of tumor cells as essentially described in Examples 1 and 2. Briefly, SCCs are isolated based on their capacity to retain CellTrace dyes (Carboxyfluorescein succinimidyl ester-CFSE or Cell Trace Violet-CTV, Invitrogen). The SCCs and FCCs are grouped as CFSE/Violet^{high}—top 10% and CFSE/Violet^{low}—bottom 10%, respectively, or FCCs in some aspects are isolated as CFSE^{low}—bottom 85% (Deleyrolle L P, et al. (2011) Brain 134:1331-43). Thus, SCCs are isolated by selecting for cells grouped as CFSE/Violet^{high}—top 10% or by removing CFSE^{low}—bottom 85% (FCCs).

RNA from SCCs is isolated as previously described (Sayour, E. J., et al. Oncoimmunology 2016, e1256527). Briefly, SCC-derived RNA is isolated using commercially available RNeasy mini kits (Qiagen) per manufacturer's instructions and cDNA libraries were generated by RT-PCR. Using a SMARTScribe Reverse Transcriptase kit (Takara), a reverse transcriptase reaction by PCR was performed on the total tumor RNA in order to generate cDNA libraries. The resulting cDNA was then amplified using Takara Advantage 2 Polymerase mix with T7/SMART and CDS III primers, with the total number of amplification cycles determined by gel electrophoresis. Purification of the cDNA was performed using a Qiagen PCR purification kit per manufacturer's instructions. In order to isolate sufficient mRNA for use in each RNA-nanoparticle vaccine, mMESAGE mMACHINE (Invitrogen) kits with T7 enzyme mix were used to perform overnight in vitro transcription on the cDNA libraries. Housekeeping genes were assessed to ensure fidelity of transcription. The resulting mRNA was then purified with a Qiagen RNeasy Maxi kit to obtain the final mRNA product. The final mRNA produce may be stored at −80° C.

Liposome Preparation: Prior to being mixed with RNA prepared from a mixed population of tumor cells, liposomes are prepared as follows:

On Day 1, the following steps were carried out in the fume hood. Water was added to a rotavapor bath. Chloroform (20 mL) was poured into a sterile, glass graduated cylinder. After opening a vial containing 1 g of DOTAP, 5 mL chloroform was added to the DOTAP vial using a glass pipette. The volume of chloroform and DOTAP was then transferred into a 1-L evaporating flask. The DOTAP vial was washed by adding a second 5-mL volume of chloroform to the DOTAP vial to dissolve any remaining DOTAP in the vial and then transferring this volume of chloroform from the DOTAP vial to the evaporating flask. This washing step was repeated 2 more times until all the chloroform in the graduated cylinder was used. The evaporating flask was then placed into the Buchi rotavapor. The water bath was turned on and adjusted to 25° C. The evaporating flask was moved downward until it touched the water bath. The rotation speed of the rotavapor was adjusted to 2. The vacuum system was turned on and adjusted to 40 mbar. After 10 minutes, the vacuum system was turned off and the chloroform was collected from the collector flask. The amount of chloroform collected was measured. Once the collector flask is repositioned, the vacuum was turned on again and the contents in the evaporating flask was allowed to dry overnight until the chloroform was completely evaporated.

On Day 2, using a sterile graduated cylinder, PBS (200 mL) was added to a new, sterile 500-mL PBS bottle maintained at room temperature. A second 500-mL PBS bottle was prepared for collecting DOTAP. The Buchi rotavapor water bath was set to 50° C. PBS (50 mL) was added into the evaporating flask using a 25-mL disposable serological pipette. The evaporating flask was positioned in the Buchi rotavapor and moved downward until ⅓ of the flask was submerged into the water bath. The rotation speed of the rotavapor was set to 2, allowed to rotate for 10 min, and then rotation was turned off. A 50-mL volume of PBS with DOTAP from the evaporating flask was transferred to the second 500 mL PBS bottle. The steps were repeated (3-times) until the entire volume of PBS in the PBS bottle was used. The final volume of the second 500 mL PBS bottle was 400 mL. The lipid solution in the second 500 mL PBS bottle was vortexed for 30 s and then incubated at 50° C. for 1 hour. During the 1 hour incubation, the bottle was vortexed every 10 min. The second 500 mL PBS bottle was allowed to rest on overnight at room temperature.

On Day 3, PBS (200 mL) was added to the second 500 mL PBS bottle containing DOTAP and PBS. The second 500 mL PBS bottle was placed into an ultrasonic bath. Water was filled in the ultrasonic bath and the second 500 mL PBS bottle was sonicated for 5 min. The extruder was washed with PBS (100 mL) and this wash step was repeated. A 0.45 μm pore filter was assembled into a filtration unit and a new (third) 500 mL PBS bottle was positioned into the output tube of the extruder. In a biological safety cabinet, the DOTAP-PBS mixture was loaded into the extruder, until about 70% of the third PBS bottle was filled. The extruder was then turned on and the DOTAP PBS mixture was added until all the mixture was run through the extruder. Subsequently, a 0.22 μm pore filter was assembled into the filtration unit and a new (third) 500 mL PBS bottle was positioned into the output tube of the extruder. The previously filtered DOTAP-PBS mixture was loaded and run again throughout. The samples comprising DOTAP lipid nanoparticles (NPs) in PBS were then stored at 4° C.

Preparation of Multilamellar RNA nanoparticles (NPs): Multilamellar RNA NPs are made by complexing DOTAP lipid NPs with RNA isolated from SCCs. Briefly, the DOTAP lipid NPs are complexed with RNA to make multilamellar RNA-NPs which were designed to have several layers of mRNA contained inside a tightly coiled liposome with a positively charged surface and an empty core. Briefly, in a safety cabinet, RNA is thawed from −80° C. and then placed on ice, and samples comprising PBS and DOTAP (e.g., DOTAP lipid NPs) are brought up to room temperature. Once components are prepared, the desired amount of RNA is mixed with PBS in a sterile tube. To the sterile tube containing the mixture of RNA and PBS, the appropriate amount of DOTAP lipid NPs is added without any physical mixing (without e.g., inversion of the tube, without vortexing, without agitation). The mixture of RNA, PBS, and DOTAP is incubated for about 15 minutes to allow multilamellar RNA-NP formation. After 15 min, the mixture is gently mixed by repeatedly inverting the tube. The mixture is then considered ready for systemic (i.e. intravenous) administration.

The amount of RNA and DOTAP lipid NPs (liposomes) used in the above preparation is pre-determined or pre-selected. In some instances, a ratio of about 15 μg liposomes per about 1 μg RNA were used. For instance, about 75 μg liposomes are used per ˜5 μg RNA or about 375 μg liposomes are used per ˜25 μg RNA. In other instances, about 7.5 μg liposomes were used per 1 μg RNA. Thus, in exemplary instances, about 1 μg to about 20 μg liposomes are used for every μg RNA used.

### Example 12

This example demonstrates a method of making an SCC-RNA-NP vaccine suitable for administration to any patient with glioblastoma.

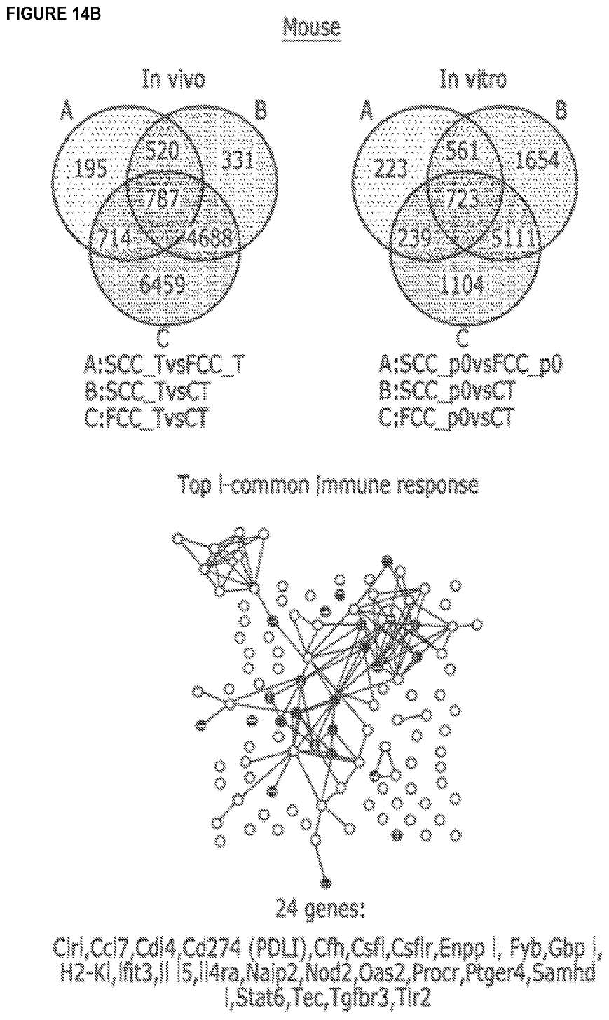

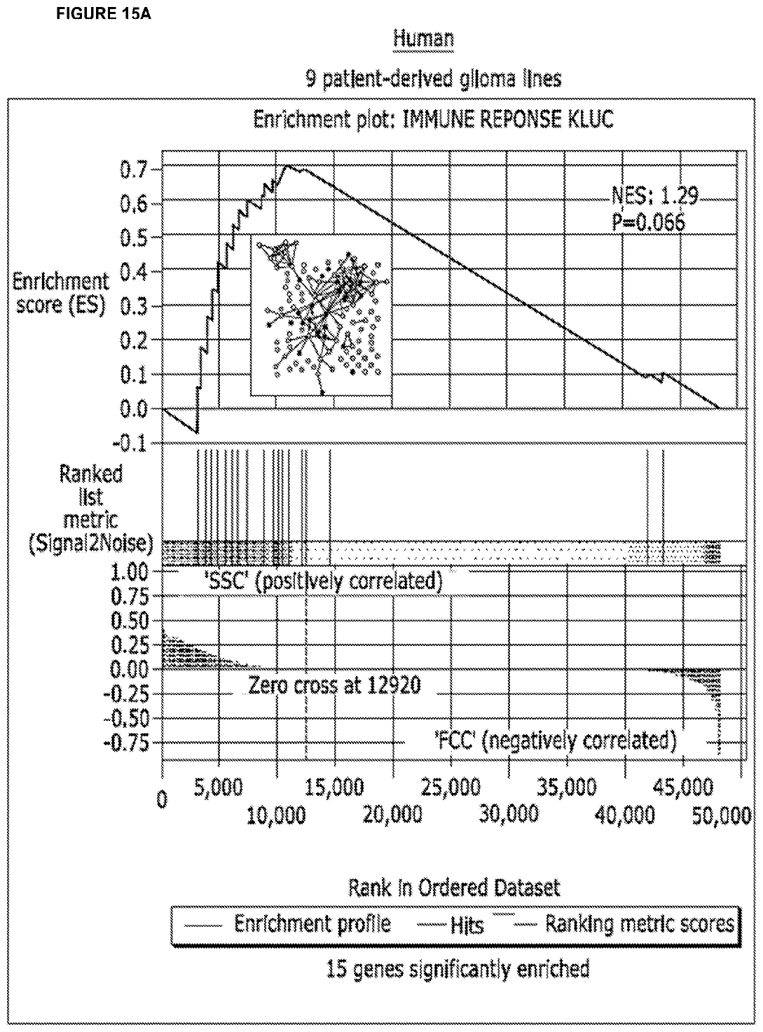

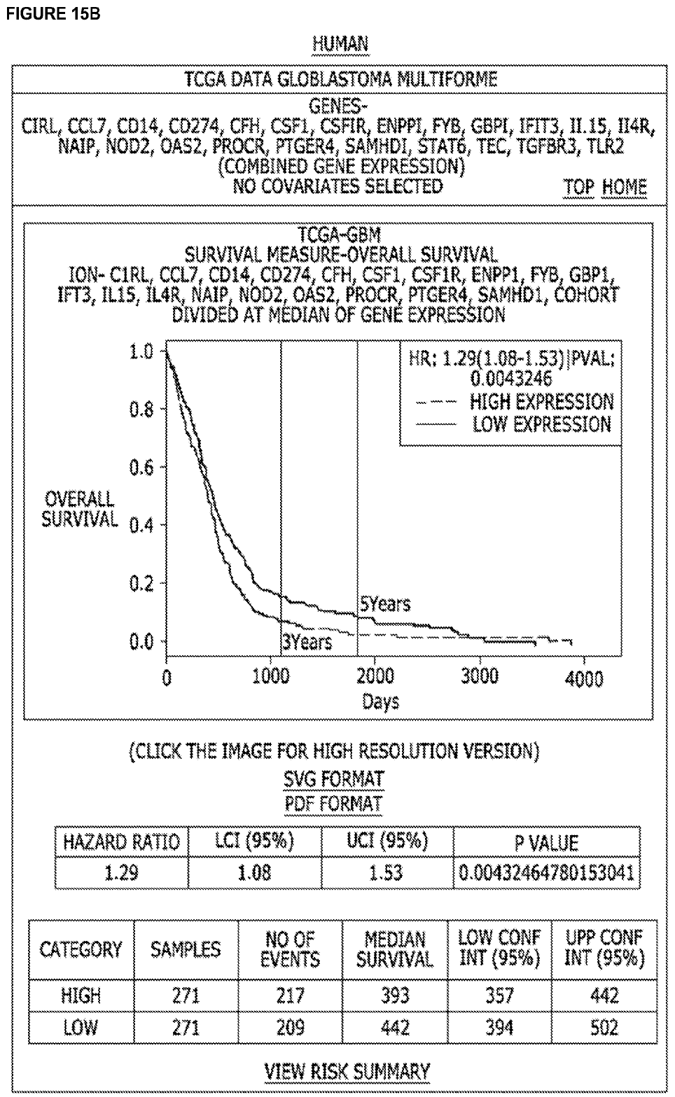

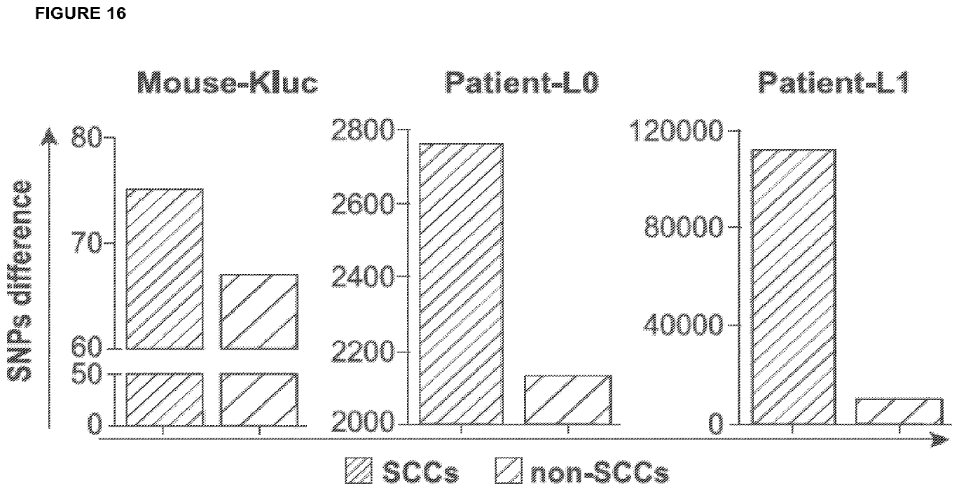

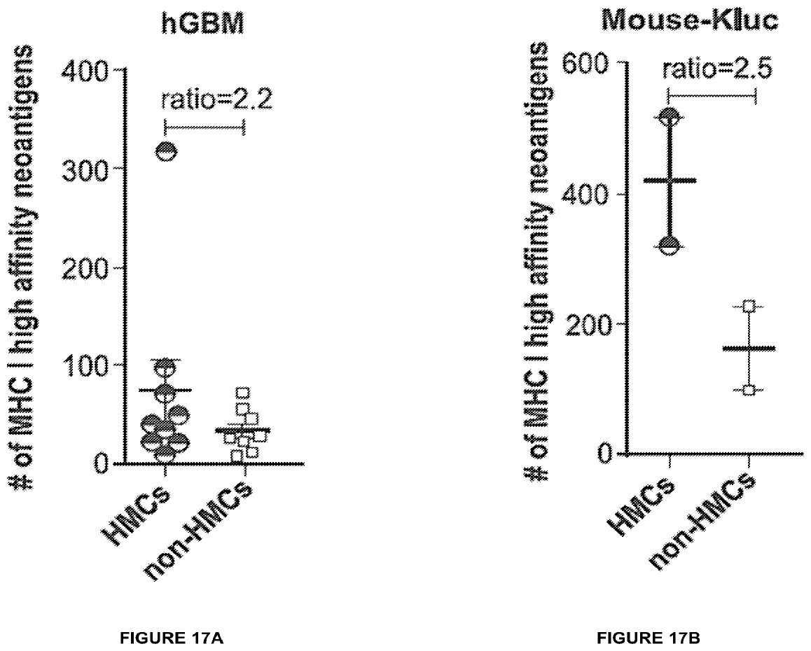

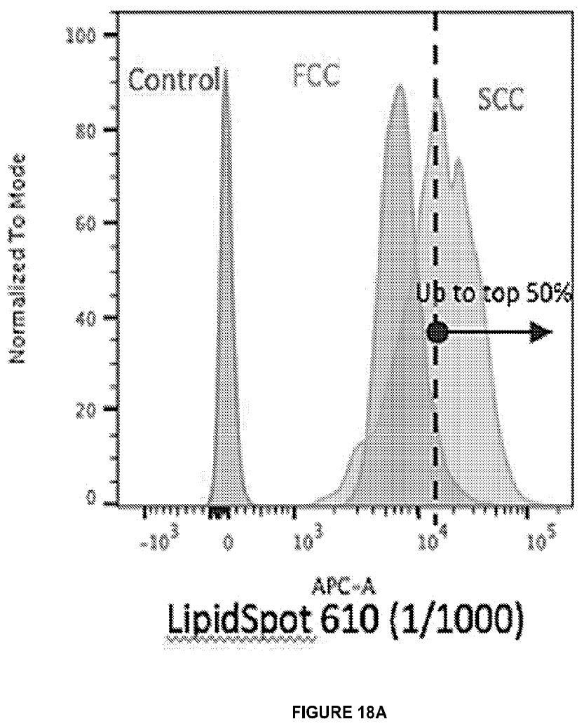

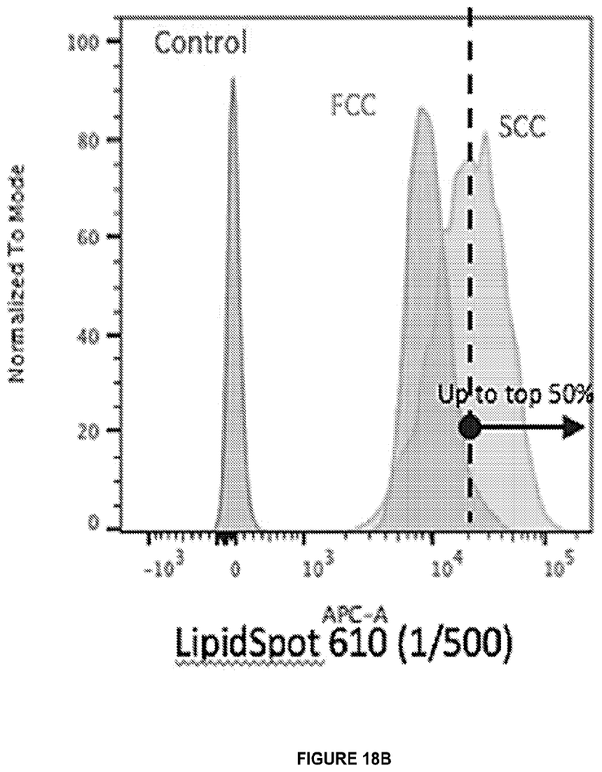

RNA sequencing analysis performed using a mouse model of glioma (KR158) revealed significant differences in the RNA population between slow and fast-cycling glioma cells (FIG. 9A). Interestingly pathways related to immune responses and processes were found to be differentially regulated between slow and fast-cycling cells both in vitro and vivo. We identified a unique immune response signature specific to the slow-cycling glioma cells commonly identified in vitro and in vivo (FIG. 14). Importantly, the majority of the genes composing the signature were also over-expressed by human slow-cycling glioma cells identified in 9 glioblastoma patients (FIG. 15). Notably, glioblastoma patients overexpressing this gene set demonstrated shorter survival, demonstrating the clinical relevance of this signature (FIG. 15).

Additional RNA sequencing evaluation enabled identification of the top 600 most important RNA specific to slow-cycling tumor cells (FIG. 14 and FIG. 20). Using nanoparticles to encapsulate any of these synthesized RNA (FIG. 20), alone or in any combination, is expected to provide a therapeutic benefit when administered to a subject with cancer, as described herein.

mRNA encoding by at least one, if not, two or more (e.g., 3, 4, 5, 6, 7, 8, 9 10, 15, 20, 25, 30, 35, 40, 45, 50, 55, 60, 65, 70, 75, 80. 85, 90, 95, 100, 150, 200, 250, 300, 350, 400, 450, 500, 550 or more (e.g., all 600)) of the genes listed in FIG. 20 are synthesized based on the sequence information available at NCBI (e.g., NCBI RefSeq listed in Gene database for given name of gene). For example, using the term “AFTPH” in the search box of the Gene database, one would select the Gene ID for Homo sapiens (Gene ID: 54812). One would click on the “NCBI Reference Sequence (RefSeq) link within the Gene ID record, and then click on one of the mRNA accession records linked by a label beginning in “NM_” followed by a series of numbers. The synthesized mRNA i then amplified from a cDNA mMessage transcription kit and cleaned up with RNeasy purification kits (Qiagen). Synthesized mRNA are stored at −80° C.

Multilamellar RNA nanoparticles are generated as essentially described in above, except that the synthesized mRNA described above is used in place of RNA prepared from SCCs. Briefly, in a safety cabinet, synthesized RNA is thawed from −80° C. and then placed on ice, and samples comprising PBS and DOTAP (e.g., DOTAP lipid NPs) are brought up to room temperature. Once components are prepared, the desired amount of RNA is mixed with PBS in a sterile tube. To the sterile tube containing the mixture of RNA and PBS, the appropriate amount of DOTAP lipid NPs is added without any physical mixing (without e.g., inversion of the tube, without vortexing, without agitation). The mixture of RNA, PBS, and DOTAP is incubated for about 15 minutes to allow multilamellar RNA-NP formation. After 15 min, the mixture is gently mixed by repeatedly inverting the tube. The mixture is then considered ready for systemic (i.e. intravenous) administration. The RNA-NPs (at a final volume of, e.g., about 25 ul to about 1000 ml) are injected into the vein of the subject with a tumor.

### Example 13

This example demonstrates that SCCs are characterized by a high lipid content and supports an alternative method to isolate slow-cycling cells.

SCCs from a murine glioma model (KR158 cells) were isolated in accordance with the procedures described in Examples 11 and 12. The SCCs were then stained with a lipid dye. In particular, live or fixed tumor cells were incubated with LipidSpot™ 610 or LipidSpot™ 488 (Biotium, Fremont, Calif., USA). The dilution of the dyes in exemplary aspects varies from 1/10 to 1/5000 and the labeling time in various instances varies from 1 minute to 24 hours. The labeling solution in some aspects is PBS or other buffer. The cell density for labeling in some aspects is from 0.1×10⁶ cells per ml of labeling solution to 20×10⁶ cells per ml of labeling solution.

As shown by the increased staining for LipidTox™ or Lipidspot™ in FIG. 18, SCCs demonstrated higher levels of lipids, suggesting that SCCs may be isolated from mixed tumor cell populations based on cellular lipid content as measured by lipid dyes (e.g., LipidSpot or LipidTox, up to top 50% most brightest cells stained with these dyes).

This example demonstrates different methods of isolating SCCs from a mixed tumor cell population. Once isolated, the SCCs may be used as a source of RNA for complexing with liposomes, as described above.

### Example 14

This example demonstrates various methods for isolating SCCs. Nucleic acid molecules may be extracted from the isolated SCCs and incorporated into RNA NPs, as described above.

Mixed populations of tumor cells are obtained as described in Example 11. Briefly, after surgical removal, the biopsied tissue is washed and mechanically dissociated before being placed in an enzymatic cocktail containing trypsin/ethylenediaminetetraacetic acid (0.05%) for 10 min at 37° C., followed by filtration through a 40-μm filter. Dead cells are quantified using trypan blue labelling and the cells are then transferred (at a density of 50 000 viable cells per ml) into neurosphere assay growth conditions. Under these culture conditions, the tumor cells generate gliomaspheres that can be serially passaged. When the gliomaspheres have reached an adequate size (˜150 μm diameter), they are dissociated using enzymatic digestion with a solution containing trypsin/ethylenediaminetetraacetic acid (0.05%) for 3-5 min. Cells are washed, counted using trypan blue to exclude dead cells and replated in fresh media supplemented with epidermal growth factor and basic fibroblast growth factor.

Isolation of SCCs from the mixed tumor population is carried out in one of the following ways. In a first method, SCCs are isolated from the mixed population of tumor cells based on proliferation rates, as described herein. Briefly, SCCs are isolated based on their capacity to retain CellTrace dyes (Carboxyfluorescein succinimidyl ester-CFSE or Cell Trace Violet-CTV, Invitrogen). The SCCs and FCCs are grouped as CFSE/Violet^{high}—top 10% and CFSE/Violet^{low}—bottom 10%, respectively, or FCCs in some aspects are isolated as CFSE^{low}—bottom 85% (Deleyrolle L P, et al. (2011) Brain 134:1331-43). Thus, SCCs are isolated by selecting for cells grouped as CFSE/Violet^{high}—top 10% or by removing CFSE^{low}—bottom 85% (FCCs).

In a second method, SCCs are isolated based on mitochondrial content. The cell-permeant MitoTracker™ (ThermoFisher Scientific, Waltham, Mass.) probes containing a mildly thiol-reactive chloromethyl moiety for labeling mitochondria is used to alternatively identify and isolate SCCs. The following dyes can be used to label live cells: Rosamine-based MitoTracker dyes, which include MitoTracker Orange CMTMRos, a derivative of tetramethylrosamine, and MitoTracker Red CMXRos, a derivative of X-rosamine. Reduced MitoTracker dyes, MitoTracker Orange CM-H2TMRos and MitoTracker Red CM-H2XRos, which are derivatives of dihydrotetramethylrosamine and dihydro-X-rosamine, respectively can also be used. The carbocyanine-based MitoTracker dyes including MitoTracker Red FM, MitoTracker Green FM dye, and MitoTracker® Deep Red FM represent additional dyes to use to stain mitochondria and identify SCCs. The concentrations of the dyes may vary from 5 nM to 1000 nM and the labeling time may vary from 1 minute to 24 hours. The labeling solution may be PBS or any buffer. The cell density for labeling may be from 0.1 million cells per ml of labeling solution to 20 million cells per ml of labeling solution.

The MitoProbe™ DilC1(5) (1,1′,3,3,3′,3′-hexamethylindodicarbo-cyanine iodide), which penetrates the cytosol of eukaryotic cells and accumulates primarily in mitochondria with active membrane potentials at concentrations below 100 nM, can be used to identify and isolate SCCs, which demonstrated greater mitochondrial membrane potential. Labeling of the cells is performed at 1 nM to 100 nM for 5 minutes to 12 hours. The labeling solution may be PBS or any buffer. The cell density for labeling may be from 0.1 million cells per ml of labeling solution to 20 million cells per ml of labeling solution. SCCs can then be identified by the brightest cells (e.g., up to the top 50% of cells that are the most bright).

In a third method, SCCs are isolated based on lipid content. In exemplary aspects, LipidSpot is used. Live of fixed cells are incubated with lipidSpot dyes including but not limited to LipidSpot 610 and LipidSpot 488. The dilutions of the dyes may vary from 1/10 to 1/5000 and the labeling time may vary from 1 minute to 24 hours. The labeling solution may be PBS or any buffer. The cell density for labeling may be from 0.1 million cells per ml of labeling solution to 20 million cells per ml of labeling solution. In other exemplary aspects, LipidTox is used. Fixed cells are incubated with lipidTox dyes including but not limited to LipidTOX Green neutral lipid stain, LipidTOX Red neutral lipid stain or LipidTOX Deep Red neutral lipid stain. The dilutions of the dyes may vary from 1/10 to 1/5000 and the labeling time may vary from 1 minute to 24 hour. The labeling solution may be PBS or any buffer. The cell density for labeling may be from 0.1 million cells per ml of labeling solution to 20 million cells per ml of labeling solution.

After SCCs are isolated, the RNA may be extracted and then amplified for use in making RNA NPs as essentially described in Example 11.

### Example 15

The RNA-NPs comprising RNA from SCCs are introduced into tumor-bearing patients as described in Example 10. This example demonstrates a method of increasing survival of subjects with tumors upon administration of RNA-NPs wherein the RNA is from SCCs.

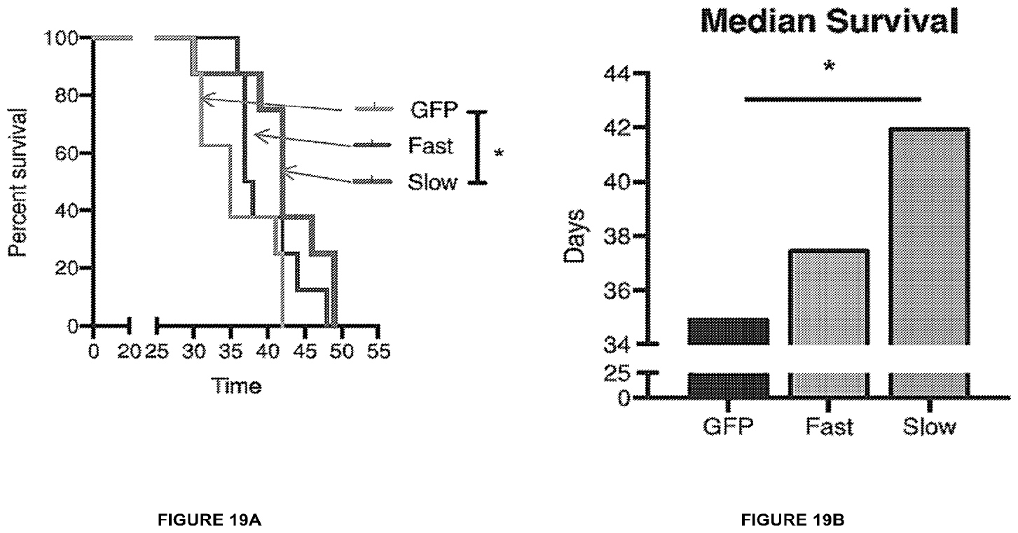

A study similar to that described in Example 10 is carried out to demonstrate the superior anti-tumor activity of RNA vaccines comprising RNA from SCCs. Briefly, KR158B cells are intracranially implanted into animals. Tumor-bearing animals are grouped into one of three groups based on treatment: (1) control RNA-NP vaccines comprising GFP RNA (control), (2) RNA NP vaccines comprising RNA isolated from fast cycling cells (fast), or (3) RNA-NP vaccines comprising RNA isolated from SCCs (slow). The results are shown in FIGS. 19A and 19B. FIG. 19A provides a Kaplan-Meier survival curve for each group and FIG. 19B represents the median survival time of each group. As shown in FIGS. 19A and 19B, only animals vaccinated with RNA-NP vaccines comprising RNA isolated from SCCs showed a significant improvement in survival compared to control.

This study is repeated with the multilamellar nanoparticles of the present disclosure which comprise RNA extracted from SSCs, as essentially described in Example 11, or synthesized RNA, as essentially described in Example 12.

### Example 16

This example describes an exemplary method of investigating the activity of personalized fusion targeted RNA-NPs against pediatric brain tumor models.

This study demonstrates the ability to synthesize fusion specific mRNA predicted from murine cell lines. The fusion protein Ppp1r13b-CKB was identified in the GL261 murine cell line by RNA-seq. The fusion breakpoint (junction of the fusion protein) was confirmed by Sanger sequencing. A portion of the fusion protein comprising the junction is:
GARQTQEQRTQRSVVNVPGEKRTENGpaaaampfsnshntqklrfpaedefpd (SEQ ID NO: 2)

In the above sequence, the sequence shown in capital letters is a portion of exon 3 of the Ppp1r13b protein and the sequence shown in lower case letters is a portion of exon 2 of the CKB protein. These sequences are joined by a new 5-amino acid sequence (PAAAA (SEQ ID NO: 3)) which is shown above as underlined letters.

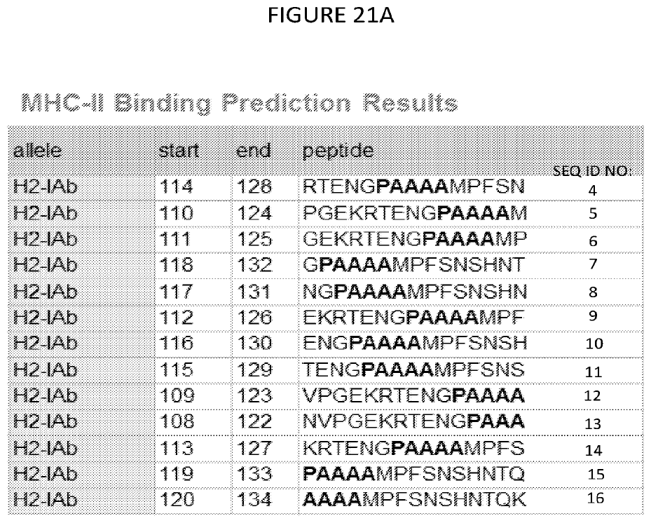

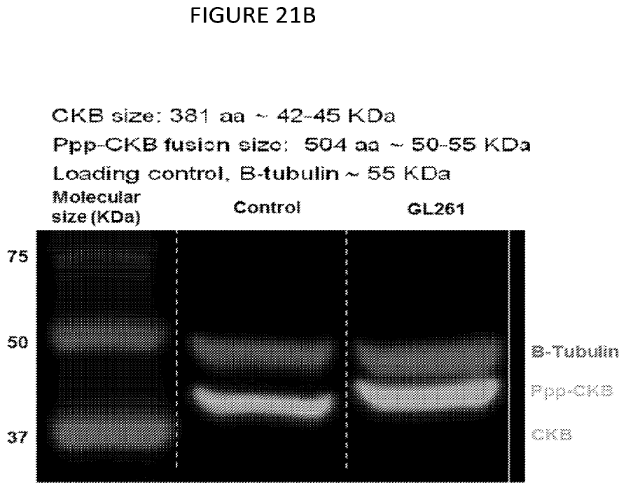

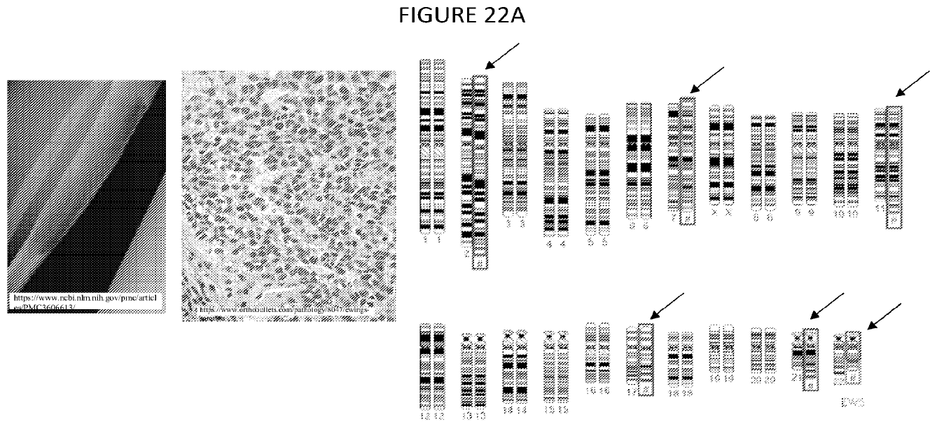

The full-length amino acid sequence of the Ppp1r13b-CKB fusion protein was analyzed for peptide binding affinity using NetMHCpan-4.0. Multiple epitopes spanning the junction (across the amino acid sequence comprising PAAAA (SEQ ID NO: 3)) were predicted to be strongly immunogenic based on MHC class II binding, suggesting that the fusion break point (junction) could a good immunogenic target. FIG. 21A lists the multiple epitopes predicted to be strongly immunogenic based on MHC class II binding.

To confirm that the fusion was not only present as mRNA but was also translated into a protein, we performed a western blot analysis using an anti-CKB antibody (FIG. 21B). As shown in FIG. 21B, the CKB band in the control (FIG. 21B, lower band in Control lane) has a size of ˜42-45KDa, as predicted. However, the CKB band in GL261 was higher than control (lower band in GL261 lane) suggesting the presence of the fusion protein, which is predicted to be around 50-55 KDa.

Subsequently, the full-length fusion protein coding sequence was cloned into a plasmid wherein fusion specific mRNA was amplified from the plasmid DNA template.

The immunogenic efficacy of fusion specific mRNA-NPs from immunocompetent murine pediatric tumor models (e.g., PTCH1 and group 3 medulloblastoma, and H3.3K27M mutant gliomas) will be evaluated. Fusion proteins will be identified by RNA seq analysis and confirmed by karyotype/fluorescent in situ hybridization. Mini-gene RNAs spanning the length of identified fusions will be synthesized. The constructs will be cloned into a modified psP73 vector (Promega). This custom vector contains a poly-A tail that is 64 nucleotides long (pSP73-Sph/A64) protecting RNA from degradation. The use of pSP73-Sph/A64 vector allows for in vitro transcription of the DNA construct due to the presence of a T7 promoter. In vitro transcription will be carried out using the mMESSAGE mMACHINE T7 transcription kit (Ambion) per manufacturer's protocol. After the generation of mRNA fusions, the nucleic acids will be complexed with nanoliposomes.

### Example 17

This example describes a study to evaluate immunogenicity of RELA targeted RNA-NPs in HLA-A2 transgenic mice.

An mRNA construct is developed spanning the full-length fusion regions in pediatric supratentorial ependymoma (e.g., C11orf95-RELA fusion), which will be cloned into plasmids for amplification and synthesis of fusion specific mRNA. This mRNA will be loaded into a NP vector, and can serve as an “off the shelf” product (i.e., personalization is not required). Immunogenicity will be assessed in humanized HLA-A2 transgenic mice vaccinated with fusion specific RNA-NPs. Antigen-specific immune response will be determined using re-stimulation assays from splenocytes of vaccinated mice co-cultured with bone marrow derived dendritic cells loaded with fusion specific mRNA.

Immunogenicity is predicted based on common human HLA haplotypes (e.g., HLA-A2) before assessing immunogenicity of fusion encapsulated RNA-NPs in HLA-A2 transgenic mice (10 mice/group). Mice are vaccinated with three weekly iv RNA-NPs injections. Immunogenicity is assessed using re-stimulation assays from harvested spleens (by ELISA and cytokine bead array, BD Biosciences) co-cultured with HLA-A2 transgenic murine bone marrow derived dendritic cells pulsed with fusion encoding mRNA.

### Example 18

This example describes a study to determine the safety and immunogenicity of fusion targeted RNA-NPs in spontaneous canine malignant glioma models.

Fusions will be determined from canine tumor biopsies. Immunogenicity/efficacy will be determined from serum/blood work and through serial imaging. Fusion specific RNA will be predicted using Star-Fusion software from sequencing analysis on canine glioma specimens. RNA-NPs will be injected intravenously to define safety and toxicity in a (3±3) dose-escalation study. The dose will begin at 0.05 mg/kg of RNA encapsulated with 0.75 mg/kg of DOTAP nanoliposomes. White blood counts, chemistries, and renal/liver function enzymes will be monitored. APCs will be assessed weekly from the peripheral blood for assessment of activation markers (e.g., PD-L1, CD80, CD86, MHCII on CD11c+ cells). T cells will also be assessed peripherally based on expression of CD3, CD8, CD4, FoxP3, and PD-1; gamma-interferon will be assessed by intracellular staining. Serum cytokines will be obtained weekly post-infusion. If dose-limiting toxicity (DLT) attributable to RNA-NPs is experienced in 1 of 3 canines, an additional 3 canines will be enrolled at the same dose. If 2 of 6 canines experiences DLT, the dose will be de-escalated in subsequently accrued canines. To assess RNA-NP efficacy, we will determine tumor size based on MRI.

Statistical analysis: Linear mixed effect models are used to compare re-stimulation assay results. Immunogenicity is compared between animals receiving fusion specific RNA-NPs and control NP vaccines using the nonparametric Mann-Whitney Test. Gehan-Breslow-Wilcoxon test is performed on Kaplan-Meier curves to assess the efficacy of RNA-NP groups (compared with tumor-bearing controls).

### Example 19

This example describes the development of RNA loaded nanoparticles for children with refractory solid tumors.

Although much of immunotherapy's promise lies in cancers with high mutational burdens, pediatric sarcomas are immunologically bland without a significant burden of mutations. However, many sarcomas including metastatic alveolar rhabdomyosarcoma are driven by aberrant fusions in FOXO1 with PAX3 or PAX7. These fusions create new epitopes that can be distinctly recognized and immunologically rejected. The tumor RNA-encoding nanoparticles (NPs) described herein simultaneously function as both vaccines and immunomodulating agents, and may be harnessed as an “off the shelf” platform to target driver fusions in pediatric sarcoma.

Intravenous administration of tumor RNA loaded NPs transfect dendritic cells (DCs) and lead to an activated T cell response for induction of anti-tumor immunity. In contrast to other formulations, RNA-NPs recruit multiple arms of the immune system (i.e., innate and adaptive), and remodel the systemic/intratumoral immune milieu, which remain potent barriers for vaccine, cellular, and checkpoint inhibiting immunotherapies. The studies described herein demonstrate successful generation of tumor-specific RNA-NP vaccines efficacious in clinically relevant canine models. These vaccines bypass MHC restriction and can be made readily available for all subjects (and not only HLA specific haplotypes); they also provide a renewable antigen resource (through the formation of a cDNA library) that can be used to continuously vaccinate patients for months/years after diagnosis.

In our preclinical studies, we uncovered that RNA-NPs elicit their immunomodulating effects through the release of interferon α (IFN-α) from plasmacytoid DCs (pDCs). IFN-α is an essential antiviral cytokine and systemic administration of RNA-NPs appears to mimic viremia for induction of near-immediate innate/adaptive immunity. This study will further investigate the immunogenicity of fusion targeted RNA-NPs targeting FOXO1 driver fusions. It is believed that RNA-NPs targeting new epitopes spanning FOXO1 fusion drivers will mediate robust antigen-specific immunity without autoimmunity. Aims of the study include investigation of the immunogenicity of RNA-NPs targeting FOXO1 fusions. The antigen-specific immunity of FOXO1 fusion targeted RNA-NPs in HLA-A2 transgenic C57131/6 mice will be assessed. Antigen-specific immune response will be determined using re-stimulation assays from splenocytes of vaccinated mice co-cultured with bone marrow derived DCs pulsed with fusion specific mRNA. Additionally, the study will further evaluate the feasibility, safety, and immunogenicity of RNA-NPs targeting fusion drivers in canines with sarcomas.

### Example 20

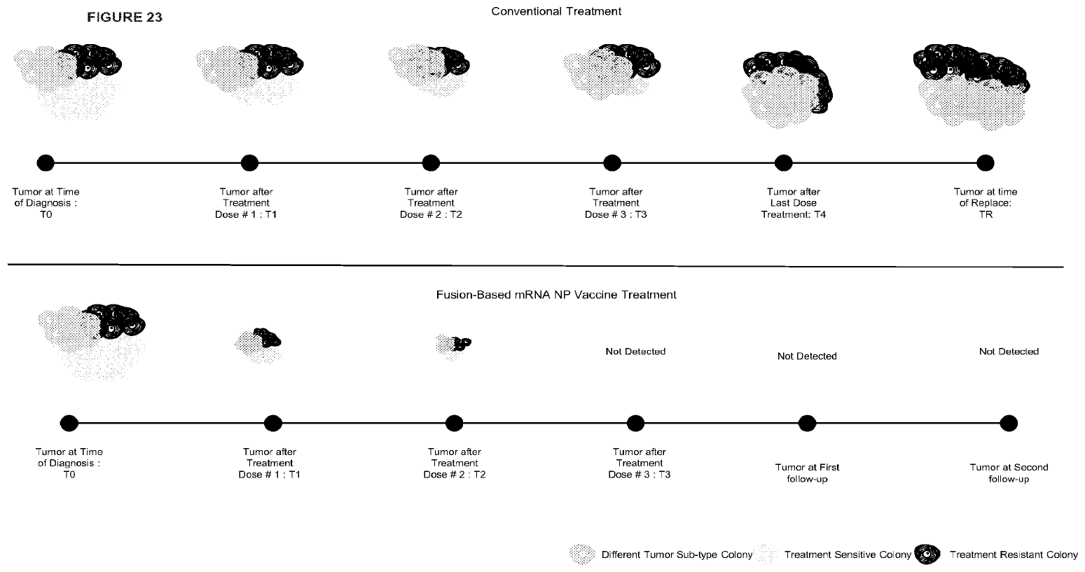

This example describes the creation of fusion protein immunotherapy vaccines. FIG. 23 illustrates a limitation associated with current immunotherapy treatment options. Conventional cancer therapy, including chemotherapy and radiotherapy, often, in hard-to-treat tumors, work on specific treatment-sensitive tumor cells, and fail to address tumor resistant colonies, as well as different colonies that are missed by biopsy. Fusion-Based mRNA NP vaccine, on the other hand, targets all the mentioned tumors' sub-colonies, as tumor cells carry that fusion protein, hence avoiding resistant colony progress and future relapse.

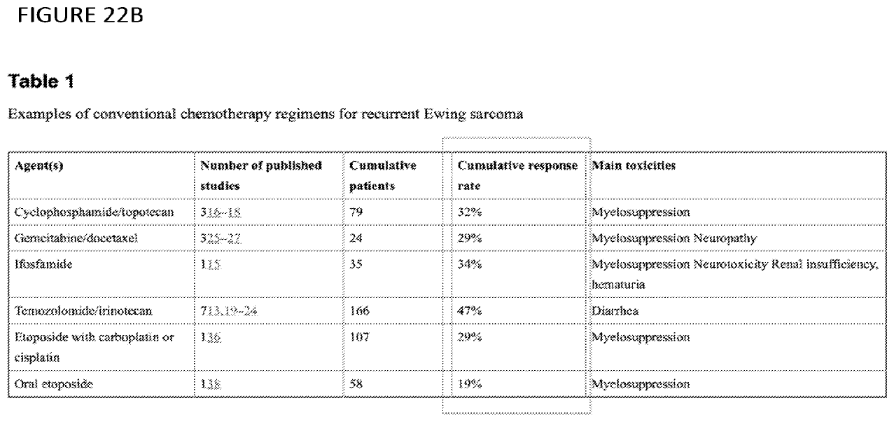

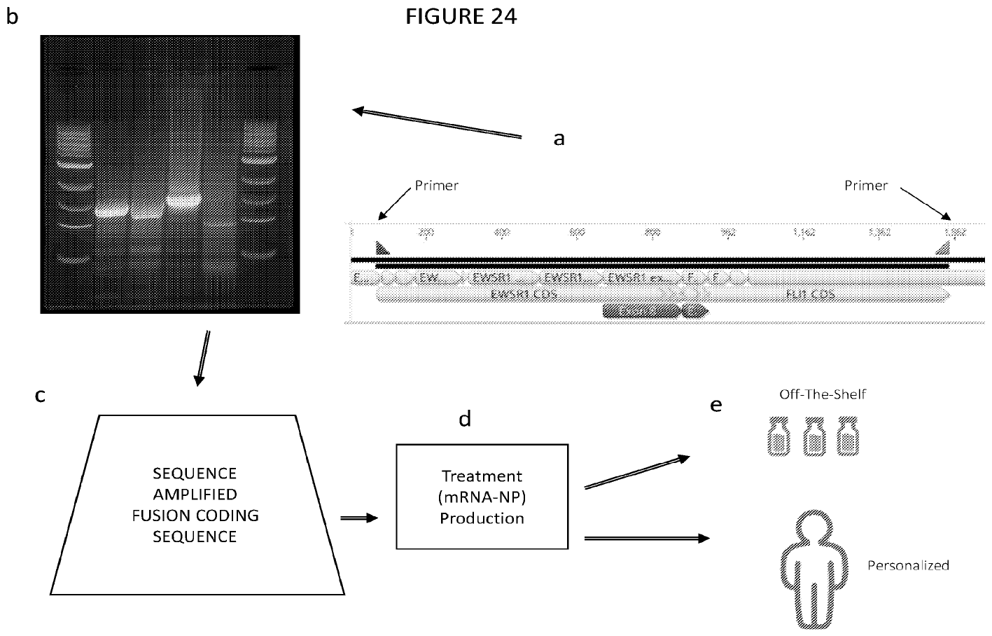

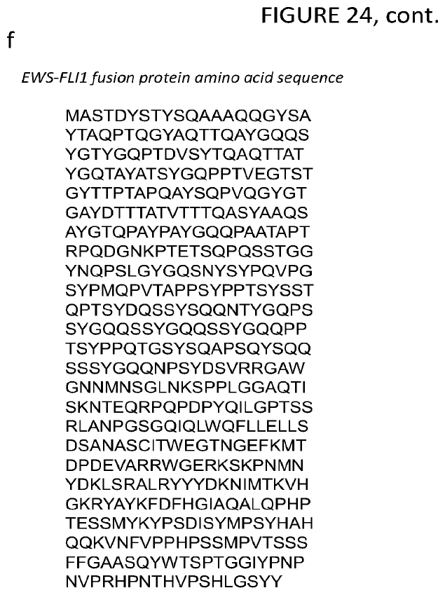

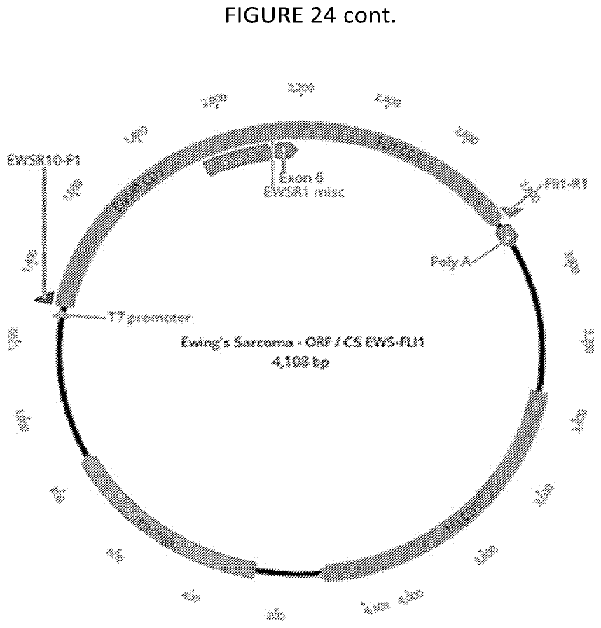

An exemplary method of treating a subject is illustrated in FIG. 24. A sample from a subject is tested to determine the presence of a fusion protein associated with a particular cancer type. Merely to illustrate, FIG. 22A describes current fusion-based tumor diagnosis and current treatment effectiveness for Ewing's Sarcoma (Onco Targets Ther. 2019; 12: 2279-2288. Published online 2019 Mar. 27. doi: 10.2147/OTT.S170585.). Particular fusion proteins may be detected by, e.g., PCR, to confirm the presence of a particular fusion protein. In FIG. 24 panel (b), primers are employed which flank the EWRS1 coding sequence and the FL1 coding sequence. If the expected amplification product is produced, the coding sequence is then sequenced. The consistency among fusion protein sequences associated with particular cancers allows the creation of “off the shelf” RNA-NP vaccines with RNA which target conserved epitopes in the fusion protein. If a subject's biopsy identifies mutant fusion proteins, a personalized RNA-NP approach may be adopted to generate RNA-NP specific to the particular mutant identified in the subject's biopsy. The platform described herein provides both an “off the shelf” option for common fusion proteins and an personalized approach for individuals having unique mutants.

While this example describes an exemplary method referencing Ewing's sarcoma, novel gene-fusions for nanoparticle-based immunotherapy treatments in the context of, e.g., alveolar rhabdomyoscarcoma, synovial sarcoma, ependymoma, and glioma (as well as others) are also contemplated. Examples of fusion proteins for use in the context of the disclosure include the following: EWSR1-FLI1 (Ewing's sarcoma—bone, soft/muscle; expected size 1515 bp); EWS-ATF (Clear cell sarcoma; 1475 bp); EML4-ALK (Non-small cell lung carcinoma; expected size 2353 bp); FIG-ROS1 (Glioblastoma; expected size 2480 bp0; C11orf95-RELA (Ependymoma and alveolar rhabdomyoscarcoma). Examples of cancer-associated fusion proteins are also set forth in FIGS. 25-28. The results described herein provide further insight into use of the platform described herein as an “off the shelf” gene-fusion immunotherapy vaccine as well as a personalized vaccine for, e.g., rare fusions.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A composition comprising a liposome comprising ribonucleic acid (RNA) molecules and a cationic lipid, wherein the RNA molecules bind to or encode an epitope of a nucleic acid encoding a fusion protein expressed by a tumor.

2. The composition of claim 1, wherein the epitope comprises a junction of the nucleic acid encoding the fusion protein.

3. The composition of claim 1 or 2, wherein the epitope encodes an amino acid sequence which binds to an MHC Class II.

4. The composition of any one of claims 1-3, wherein the tumor is a solid tumor, optionally, a refractory solid tumor.

5. The composition of any one claims 1-4, wherein the tumor is a brain tumor.

6. The composition of any one of claims 1-5, wherein the tumor is a sarcoma.

7. The composition of any one claims 1-6, wherein the tumor is a resistant supratentorial ependymoma or a metastatic alveolar rhabdomyosarcoma.

8. The composition of any one of claims 1-7, wherein the fusion protein is a C11orf95-RELA fusion protein or a fusion protein described herein or in Parker and Zhang, Chin J Cancer 32(11): 594-603 (2013); Ding et al., In J Mol Sci 19(1): 177 (2018), Wener et al., Molecular Cancer 17, article number 28 (2018); Yu et al., Scientific Reports 9, article number 1074 (2019).

9. The composition of any one of claims 1-8, comprising a nanoparticle comprising a positively-charged surface and an interior comprising (i) a core and (ii) at least two nucleic acid layers, wherein each nucleic acid layer is positioned between a cationic lipid bilayer.

10. The composition of claim 9, wherein the nanoparticle comprises at least three nucleic acid layers, each of which is positioned between a cationic lipid bilayer.

11. The composition of claim 10, wherein the nanoparticle comprises at least four nucleic acid layers, each of which is positioned between a cationic lipid bilayer.

12. The composition of claim 11, wherein the nanoparticle comprises five or more nucleic acid layers, each of which is positioned between a cationic lipid bilayer.

13. The composition of any one of claims 9-12, wherein the outermost layer of the nanoparticle comprises a cationic lipid bilayer.

14. The composition of any one of claims 9-13, wherein the core comprises a cationic lipid bilayer.

15. The composition of any one of claims 9-14, wherein the diameter of the nanoparticle is about 50 nm to about 250 nm in diameter, optionally, about 70 nm to about 200 nm in diameter.

16. The composition of any one of claims 9-15, wherein the nanoparticle comprises a zeta potential of about 40 mV to about 60 mV, optionally, about 45 mV to about 55 mV.

17. The composition of claim 16, comprising a zeta potential of about 50 mV.

18. The composition of any one of claims 1-17, comprising nucleic acid molecules and cationic lipid at a ratio of about 1 to about 5 to about 1 to about 20, optionally, about 1 to about 15 or about 1 to about 7.5.

19. The composition of any one claims 1-18, wherein the cationic lipid is DOTAP or DOTMA.

20. The composition of any one claims 1-19, wherein the RNA molecules are mRNA.

21. A method of making a nanoparticle comprising a positively-charged surface and an interior comprising (i) a core and (ii) at least two nucleic acid layers, wherein each nucleic acid layer is positioned between a cationic lipid bilayer, said method comprising:
(A) mixing nucleic acid molecules and liposomes at a RNA: liposome ratio of about 1 to about 5 to about 1 to about 20, optionally, about 1 to about 15, to obtain RNA-coated liposomes, wherein the liposomes are made by a process of making liposomes comprising drying a lipid mixture comprising a cationic lipid and an organic solvent by evaporating the organic solvent under a vacuum; and
(B) mixing the RNA-coated liposomes with a surplus amount of liposomes,
wherein the RNA binds to or encodes an epitope of a nucleic acid encoding a fusion protein expressed by a tumor.

22. The method of claim 21, wherein the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent.

23. The method of claim 21 or 22, wherein the process of making liposomes further comprises rehydrating the lipid mixture with a rehydration solution to form a rehydrated lipid mixture and then agitating, resting, and sizing the rehydrated lipid mixture.

24. The method of claim 23, wherein sizing the rehydrated lipid mixture comprises sonicating, extruding and/or filtering the rehydrated lipid mixture.

25. The method of any one of claims 21-24, wherein the nanoparticle has a zeta potential of about 40 mV to about 60 mV, optionally, about 45 mV to about 55 mV.

26. A nanoparticle made by the method of any one of claims 16-25.

27. A cell comprising a nanoparticle as described in any one of claims 1-20 or according to claim 26.

28. The cell of claim 27, which is an antigen-presenting cell (APC), optionally, a dendritic cell (DC).

29. A population of cells, wherein at least 50% of the population are cells according to any one of claim 27 or 28.

30. A pharmaceutical composition comprising a plurality of nanoparticles according to any one of claims 1-50 or claim 26 and a pharmaceutically acceptable carrier, diluent, or excipient.

31. A method of increasing an immune response against a tumor in a subject, comprising administering to the subject the pharmaceutical composition of claim 30.

32. The method of claim 31, wherein the RNA molecules are mRNA.

33. The method of claim 31 or 32, wherein the composition is systemically administered to the subject.

34. The method of claim 33, wherein the composition is administered intravenously.

35. The method of any one of claims 31-34, wherein the pharmaceutical composition is administered in an amount which is effective to activate dendritic cells (DCs) in the subject.

36. The method of any one of claims 31-35, wherein the immune response is a T cell-mediated immune response.

37. A method of treating a subject with a disease, comprising administering to the subject a pharmaceutical composition of claim 30 in an amount effective to treat the disease in the subject.

38. The method of claim 37, wherein the subject has cancer or a tumor.

39. The method of claim 48, wherein the pharmaceutical composition is administered intravenously to the patient.

40. The method of any one of claims 37-39, wherein the patient is a pediatric patient.

41. Use of the pharmaceutical composition of claim 30 for increasing an immune response against a tumor in a subject or treating a subject with a disease, such as cancer.

42. Use of the composition of any one of claims 1-20 in the preparation of a medicament for increasing an immune response against a tumor in a subject or treating a subject with a disease, such as cancer.

43. A nanoparticle comprising a positively-charged surface and an interior comprising (i) a core and (ii) at least two nucleic acid layers, wherein each nucleic acid layer is positioned between a cationic lipid bilayer, and nucleic acid molecules in the nucleic acid layers comprise a sequence of a nucleic acid molecule expressed by slow-cycling cells (SCCs).

44. The nanoparticle of claim 43, comprising at least three nucleic acid layers, each of which is positioned between a cationic lipid bilayer.

45. The nanoparticle of claim 44, comprising at least four nucleic acid layers, each of which is positioned between a cationic lipid bilayer.

46. The nanoparticle of claim 45, comprising five or more nucleic acid layers, each of which is positioned between a cationic lipid bilayer.

47. The nanoparticle of any one of claims 43-46, wherein the outermost layer of the nanoparticle comprises a cationic lipid bilayer.

48. The nanoparticle of any one of claims 43-47, wherein the core comprises a cationic lipid bilayer.

49. The nanoparticle of any one of claims 43-48, wherein the diameter of the nanoparticle is about 50 nm to about 250 nm in diameter, optionally, about 70 nm to about 200 nm in diameter.

50. The nanoparticle of any one of claims 43-4958, comprising a zeta potential of about 40 mV to about 60 mV, optionally, about 45 mV to about 55 mV.

51. The nanoparticle of claim 50, comprising a zeta potential of about 50 mV.

52. The nanoparticle of any one claims 43-51, comprising nucleic acid molecules and cationic lipid at a ratio of about 1 to about 5 to about 1 to about 20, optionally, about 1 to about 15 or about 1 to about 7.5.

53. The nanoparticle of any one claims 43-52, wherein the cationic lipid is DOTAP or DOTMA.

54. The nanoparticle of any one claims 43-53, wherein the nucleic acid molecules are RNA molecules.

55. The nanoparticle of claim 54, wherein the RNA molecules are mRNA.

56. The nanoparticle of claim 55, wherein mRNA is amplified transcribed mRNA prepared from cDNA made from mRNA isolated from SCCs isolated from a mixed tumor cell population obtained from a subject with a tumor.

57. The nanoparticle of claim 56, wherein the tumor is a glioblastoma.

58. The nanoparticle of any one of claims 43-57, comprising nucleic acid molecules encoded by at least one gene listed in FIG. 20.

59. The nanoparticle of claim 58, comprising nucleic acid molecules encoded by at least or about 2, 3, 4, 5, 6, 7, 8, 9, 10, 11, 12, 13, 14, 15, 16, 17, 18, 19, or 20 genes listed in FIG. 20.

60. The nanoparticle of claim 58, comprising nucleic acid molecules encoded by more than about 50, 60, 70, 80, 90, 100 genes listed in FIG. 20.

61. The nanoparticle of claim 58, comprising nucleic acid molecules encoded by at least or about 200, 300, 400, 500, or 600 genes listed in FIG. 20.

62. The nanoparticle of any one claims 43-62, wherein comprising nucleic acid molecules and cationic lipid at a nucleic acid molecule: cationic lipid ratio of about 1 to about 5 to about 1 to about 20, optionally, about 1 to about 15 or about 1 to about 7.5.

63. A method of making a nanoparticle comprising a positively-charged surface and an interior comprising (i) a core and (ii) at least two nucleic acid layers, wherein each nucleic acid layer is positioned between a cationic lipid bilayer, wherein the nanoparticle comprises nucleic acid molecules comprising a sequence of a nucleic acid molecule expressed by slow-cycling cells (SCCs), said method comprising:
(A) mixing nucleic acid molecules comprising a sequence of a nucleic acid molecule expressed by slow1cycling cells (SCCs) and liposomes made by a process of making liposomes comprising drying a lipid mixture comprising a cationic lipid and an organic solvent by evaporating the organic solvent under a vacuum, wherein the nucleic acid molecules and the liposomes are mixed at a nucleic acid: liposome ratio of about 1 to about 5 to about 1 to about 20, optionally, about 1 to about 15, to obtain nucleic acid-coated liposomes; and
(B) mixing the nucleic acid-coated liposomes with a surplus amount of liposomes.

64. The method of claim 63, wherein the nucleic acid molecules are RNA.

65. The method of claim 63 or claim 64, further comprising extracting RNA from the isolated SCCs.

66. The method of any one of claims 63-65, further comprising preparing mRNA by amplifying transcribed mRNA from cDNA libraries generated by reverse transcription from total RNA isolated from SCCs.

67. The method of any one of claims 63-66, further comprising isolating SCCs from a mixed tumor cell population using a flow cytometer.

68. The method of claim 67, comprising isolating SCCs from a mixed tumor cell population based on proliferation rate, mitochondrial content, lipid content or a combination thereof.

69. The method of claim 68, comprising isolating the SCCs from a mixed tumor cell population based on proliferation rate using a dye that covalently binds to free amines of intracellular proteins, optionally, wherein the dye is a carboxyfluorescein succinimidyl ester (CFSE) dye, a Carboxyfluorescein diacetate (CFDA) dye, a Carboxyfluorescein diacetate succinimidyl ester (CFDA-SE) dye, a CellTrace™ Proliferation dye (e.g., a CellTrace™ Violet (CTV) dye), a CellVue® Claret dye, a PKH26 dye, or an e-Fluor™ Proliferation dye.

70. The method of claim 67, comprising isolating the SCCs from a mixed tumor cell population based on mitochondrial content using a dye that binds to thiol groups in the mitochondria, optionally, wherein the dye comprises a thiol-reactive moiety, optionally, a thiol-reactive chloromethyl moiety.

71. The method of claim 67, comprising isolating the SCCs from a mixed tumor cell population based on lipid content using a dye that stains lipid droplets, optionally, wherein the dye is LipidTox or LipidSpot dye.

72. The method of any one of claims 63-71, wherein the nucleic acid molecules are encoded by at least or about 1, 2, 3, 4, 5, 6, 7, 8, 9, 10, 11, 12, 13, 14, 15, 16, 17, 18, 19, or 20 genes listed in FIG. 20.

73. The method of claim 72, wherein the nucleic acid molecules are encoded by more than about 50, 60, 70, 80, 90, 100 genes listed in FIG. 20.

74. The method of claim 72, wherein the nucleic acid molecules are encoded by at least or about 200, 300, 400, 500, or 600 genes listed in FIG. 20.

75. The method of any one of claims 63-74, wherein the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent.

76. The method of any one of claims 63-75, wherein the process of making liposomes further comprises rehydrating the lipid mixture with a rehydration solution to form a rehydrated lipid mixture and then agitating, resting, and sizing the rehydrated lipid mixture.

77. The method of claim 76, wherein sizing the rehydrated lipid mixture comprises sonicating, extruding and/or filtering the rehydrated lipid mixture.

78. The method of any one of claims 63-77, wherein the nanoparticle has a zeta potential of about 40 mV to about 60 mV, optionally, about 45 mV to about 55 mV.

79. A nanoparticle made by the method of any one of claims 63-78.

80. A cell comprising a nanoparticle as described in any one of claims 43-62 or according to claim 79.

81. The cell of claim 80, which is an antigen presenting cell (APC), optionally, a dendritic cell (DC).

82. A population of cells, wherein at least 50% of the population are cells according to any one of claim 80 or 81.

83. A pharmaceutical composition comprising a plurality of nanoparticles according to any one of claims 43-62 or claim 79 and a pharmaceutically acceptable carrier, diluent, or excipient.

84. A method of increasing an immune response against a tumor in a subject, comprising administering to the subject the pharmaceutical composition of claim 83.

85. The method of claim 84, wherein the nucleic acid molecules are mRNA.

86. The method of claim 84 or 85, wherein the composition is systemically administered to the subject.

87. The method of claim 86, wherein the composition is administered intravenously.

88. The method of any one of claims 84-87, wherein the pharmaceutical composition is administered in an amount which is effective to activate dendritic cells (DCs) in the subject.

89. The method of any one of claims 84-88, wherein the immune response is a T cell-mediated immune response.

90. The method of claim 89, wherein the T cell-mediated immune response comprises activity by tumor infiltrating lymphocytes (TILs).

91. A method of delivering RNA molecules to an intra-tumoral microenvironment, lymph node, and/or a reticuloendothelial organ, comprising administering to the subject a pharmaceutical composition of claim 83.

92. The method of claim 91, wherein the reticuloendothelial organ is a spleen or liver.

93. A method of treating a subject with a disease, comprising administering to the subject a pharmaceutical composition of claim 83 in an amount effective to treat the disease in the subject.

94. A method of treating a subject with a disease, comprising administering to the subject the cells of claim 80 in an amount effective to treat the disease in the subject.

95. The method of claim 93 or 94, wherein the subject has a cancer or a tumor.

96. The method of claim 95, wherein the tumor is a malignant brain tumor, optionally, a glioblastoma, medulloblastoma, diffuse intrinsic pontine glioma, or a peripheral tumor with metastatic infiltration into the central nervous system.

97. Use of the pharmaceutical composition of claim 83 for increasing an immune response against a tumor in a subject or treating a subject with a disease, such as cancer.

98. Use of the composition of any one of claims 43-62 in the preparation of a medicament for increasing an immune response against a tumor in a subject or treating a subject with a disease, such as cancer.


SEQUENCE LISTING (17 sequences)

SEQ ID NO: 1 - DNA, 19 bp, Artificial Sequence
  Syntheic
cttgactgataattaccgt

SEQ ID NO: 2 - protein, 53 aa, Artificial Sequence
  Syntheic
GARQTQEQRTQRSVVNVPGEKRTENGPAAAAMPFSNSHNTQKLRFPAEDEFPD

SEQ ID NO: 3 - protein, 5 aa, Artificial Sequence
  Syntheic
PAAAA

SEQ ID NO: 4 - protein, 15 aa, Artificial Sequence
  Syntheic
RTENGPAAAAMPFSN

SEQ ID NO: 5 - protein, 15 aa, Artificial Sequence
  Syntheic
PGEKRTENGPAAAAM

SEQ ID NO: 6 - protein, 15 aa, Artificial Sequence
  Syntheic
GEKRTENGPAAAAMP

SEQ ID NO: 7 - protein, 15 aa, Artificial Sequence
  Syntheic
GPAAAAMPFSNSHNT

SEQ ID NO: 8 - protein, 15 aa, Artificial Sequence
  Syntheic
NGPAAAAMPFSNSHN

SEQ ID NO: 9 - protein, 15 aa, Artificial Sequence
  Syntheic
EKRTENGPAAAAMPF

SEQ ID NO: 10 - protein, 15 aa, Artificial Sequence
  Syntheic
ENGPAAAAMPFSNSH

SEQ ID NO: 11 - protein, 15 aa, Artificial Sequence
  Syntheic
TENGPAAAAMPFSNS

SEQ ID NO: 12 - protein, 15 aa, Artificial Sequence
  Syntheic
VPGEKRTENGPAAAA

SEQ ID NO: 13 - protein, 15 aa, Artificial Sequence
  Syntheic
NVPGEKRTENGPAAA

SEQ ID NO: 14 - protein, 15 aa, Artificial Sequence
  Syntheic
KRTENGPAAAAMPFS

SEQ ID NO: 15 - protein, 15 aa, Artificial Sequence
  Syntheic
PAAAAMPFSNSHNTQ

SEQ ID NO: 16 - protein, 15 aa, Artificial Sequence
  Syntheic
AAAAMPFSNSHNTQK

SEQ ID NO: 17 - protein, 504 aa, Artificial Sequence
  Syntheic
MASTDYSTYSQAAAQQGYSAYTAQPTQGYAQTTQAYGQQSYGTYGQPTDVSYTQAQTTATYGQTAYATSYGQPPTVEGTSTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYGQQNPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY